(19) United States
(12) Patent Application Publication
(10) Pub. No.: US 2008/0070954 A1
(43) Pub. Date: Mar. 20, 2008

(54) Acylurea Connected And Sulfonylurea Connected Hydroxamates

(75) Inventors: Ze-Yi Lim, Singapore (SG); Haishan Wang, Singapore (SG); Yan Zhou, Cleveland, OH (US)

Correspondence Address: CONNOLLY BOVE LODGE & HUTZ LLP, 1875 EYE STREET, N.W., SUITE 1100, WASHINGTON, DC, 20036, US

(73) Assignee: S*BIO PTE LTD., Singapore (SG)

(21) Appl. No.: 10/577,462

(22) PCT Filed: Oct. 26, 2004
(86) PCT No.: PCT/SG04/00353

§ 371 Date: Sep. 27, 2007

Related U.S. Application Data
(60) Provisional application No. 60/514,013, filed on Oct. 27, 2003.

(51) Int. Cl. A61K 31/4402; A61K 31/19; A61P 25/00; A61P 29/00; A61P 31/00; A61P 7/00; C07D 209/18; C12N 5/06; C12N 9/00; C07D 211/04; C07C 259/06; A61P 35/00; A61P 3/00; A61P 27/00; A61K 31/404

(52) U.S. Cl. 514/357; 435/183; 435/375; 514/419; 514/575; 546/332; 548/495; 562/623

(57) ABSTRACT

The present invention relates to hydroxamate compounds which are inhibitors of histone deacetylase. More particularly, the present invention relates to acylurea/sulfonylurea containing compounds and methods for their preparation. These compounds may be useful as medicaments for the treatment of proliferative disorders as well as other diseases involving, relating to or associated with enzymes having histone deacetylase activities.

### FIELD OF THE INVENTION

The present invention relates to hydroxamate compounds that are inhibitors of histone deacetylase. More particularly, the present invention relates to acylurea or sulfonylurea containing compounds and methods for their preparation. These compounds may be useful as medicaments for the treatment of proliferative disorders as well as other diseases involving, relating to or associated with enzymes having histone deacetylase activities.

### BACKGROUND OF THE INVENTION

Local chromatin architecture is generally recognized as an important factor in the regulation of gene expression. The architecture of chromatin, a protein-DNA complex, is strongly influenced by post-translational modifications of the histones which are the protein components. Reversible acetylation of histones is a key component in the regulation of gene expression by altering the accessibility of transcription factors to DNA. In general, increased levels of histone acetylation are associated with increased transcriptional activity, whereas decreased levels of acetylation are associated with repression of gene expression [Wade P. A. Hum. Mol. Genet. 10, 693-698 (2001), De Ruijter A. J. M. et al, Biochem. J., 370, 737-749 (2003)]. In normal cells, histone deacetylases (HDACs) and histone acetyltransferase together control the level of acetylation of histones to maintain a balance. Inhibition of HDACs results in the accumulation of acetylated histones, which results in a variety of cell type dependent cellular responses, such as apoptosis, necrosis, differentiation, cell survival, inhibition of proliferation and cytostasis.

Inhibitors of HDAC have been studied for their therapeutic effects on cancer cells. For example, suberoylanilide hydroxamic acid (SAHA) is a potent inducer of differentiation and/or apoptosis in murine erythroleukemia, bladder, and myeloma cell lines [Richon V. M. et al, Proc. Natl. Acad. Sci. USA, 93: 5705-5708 (1996), Richon V. M. et al, Proc. Natl. Acad. Sci. USA, 95: 3003-3007 (1998)]. SAHA has been shown to suppress the growth of prostate cancer cells in vitro and in vivo [Butler L. M. et al, Cancer Res. 60, 5165-5170 (2000)]. Other inhibitors of HDAC that have been widely studied for their anti-cancer activities are trichostatin A (TSA) and trapoxin B [Yoshida M. et al, J. Biol. Chem., 265, 17174 (1990), Kijima M. et al, J. Biol. Chem., 268, 22429 (1993)]. Trichostatin A is a reversible inhibitor of mammalian HDAC. Trapoxin B is a cyclic tetrapeptide, which is an irreversible inhibitor of mammalian HDAC. However, due to the in vivo instability of these compounds they are less desirable as anti-cancer drugs. Recently, other small molecule HDAC inhibitors have become available for clinical evaluation [U.S. Pat. No. 6,552,065]. Additional HDAC inhibiting compounds have been reported in the literature [Bouchain G. et al, J. Med. Chem., 46, 820-830 (2003)] and patents [WO 03/066579A2, WO 01/38322 A1]. The in vivo activity of such inhibitors can be directly monitored by their ability to increase the amount of acetylated histones in the biological sample. HDAC inhibitors have been reported to interfere with neurodegenerative processes, for instance, HDAC inhibitors arrest polyglutamine-dependent neurodegeneration [Nature, 413(6857): 739-43, 18 Oct., 2001]. In addition, HDAC inhibitors have also been known to inhibit production of cytokines such as TNF, IFN, IL-1 which are known to be implicated in inflammatory diseases and/or immune system disorders. [J. Biol. Chem. 1990; 265(18): 10230-10237; Science, 1998, 281: 1001-1005; Dinarello C. A. and Moldawer L. L. Proinflammatory and anti-inflammatory cytokines in rheumatoid arthritis. A primer for clinicians. 2^{nd} Edition, Amergen Inc., 2000].

Nevertheless, there is still a need to provide further HDAC inhibitors that would be expected to have useful, improved pharmaceutical properties in the treatment of diseases such as cancer, neurodegenerative diseases and inflammatory and/or immune system disorders.

### SUMMARY OF THE INVENTION

In one aspect the present invention provides compounds of the Formula (I)

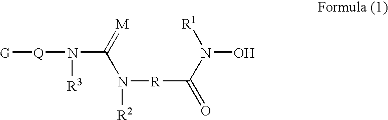

wherein

R is a linking moiety;

R¹ is selected from the group consisting of H, C₁-C₆ alkyl and acyl;

M is selected from the group consisting of O, S, NH, NR⁴, NOH and NOR⁴;

R² is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴, NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted;

or

R² together with the nitrogen to which it is attached and a portion of R form an optionally substituted heterocycloalkyl group;

R³ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴, NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted;

Q is selected from the group consisting of —S(O)₂—, —C(═O)— and —C(═S)—;

G is selected from the group consisting of optionally substituted alkyl, optionally substituted cycloalkyl, optionally substituted aryl, optionally substituted heteroaryl, optionally substituted heterocycloalkyl, optionally substituted arylalkyl, and optionally substituted heteroarylalkyl;

each R⁴ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl; each of which may be optionally substituted;

or a pharmaceutically acceptable salt or prodrug thereof.

In one preferred embodiment the present invention provides compounds having the Formula (2)

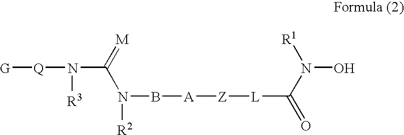

wherein

R¹ is selected from the group consisting of H, C₁-C₆ alkyl and acyl;

L is a single bond or is a C₁-C₅ hydrocarbon chain which may contain 0 to 2 multiple bonds independently selected from double bonds and triple bonds and wherein, the chain may optionally be interrupted by at least one of —O—, —, —S(O)— and —S(O)₂— and the chain may optionally be substituted with one or more substituents independently selected from the group consisting of C₁-C₄ alkyl;

Z is selected from the group consisting of a single bond, N(R¹), O, S, S(O) and S(O)₂;

A is selected from the group consisting of a single bond, optionally substituted arylene, optionally substituted heteroarylene, optionally substituted cycloalkylene and optionally substituted heterocycloalkylene;

B is selected from the group consisting of a single bond, optionally substituted aminoacyl, optionally substituted arylene, optionally substituted heteroarylene, optionally substituted arylalkylene, optionally substituted heteroarylalkylene, optionally substituted alkylarylene, optionally substituted alkylheteroarylene, optionally substituted C₁-C₃ alkylene, optionally substituted heteroalkylene, optionally substituted cycloalkylene, optionally substituted heterocycloalkylene and optionally substituted —(CH₂)ₘ—C(O)—N(R⁴)—(CH₂)ₙ—, wherein n is an integer from 0 to 6, m is an integer from 0 to 6;

M is selected from the group consisting of O, S, NH, NR⁴, NOH and NOR⁴;

R² is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted; or

R² together with the nitrogen to which it is attached and a portion of B form an optionally substituted heterocycloalkyl group;

R³ is independently selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴, NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted;

Q is selected from the group consisting of —S(O)₂—, —C(═O)— and —C(═S)—;

G is selected from the group consisting of optionally substituted aryl, optionally substituted heteroaryl, optionally substituted alkyl, optionally substituted cycloalkyl, optionally substituted heterocycloalkyl, optionally substituted arylalkyl and optionally substituted heteroarylalkyl;

each R⁴ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl; each of which may be optionally substituted;

or a pharmaceutically acceptable salt or prodrug thereof.

In a particularly preferred embodiment of the compounds of Formula (2) are compounds of Formula (2a)

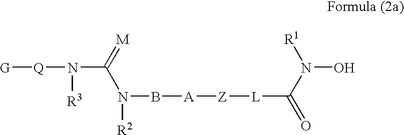

wherein

R¹ is selected from the group consisting of H, C₁-C₆ alkyl and acyl;

L is a single bond or is a C₁-C₅ hydrocarbon chain which may contain 0 to 2 multiple bonds independently selected from double bonds and triple bonds and wherein, the chain may optionally be interrupted by at least one of —O—, —S—, —S(O)— and —S(O)₂— and the chain may optionally be substituted with one or more substituents independently selected from the group consisting of C₁-C₄ alkyl;

Z is selected from the group consisting of a single bond, N(R¹), O, S, S(O) and S(O)₂;

A is selected from the group consisting of a single bond, optionally substituted arylene, optionally substituted heteroarylene, optionally substituted cycloalkylene and optionally substituted heterocycloalkylene;

B is selected from the group consisting of a single bond, optionally substituted aminoacyl, optionally substituted arylene, optionally substituted heteroarylene, optionally substituted arylalkylene, optionally substituted heteroarylalkylene, optionally substituted alkylarylene, optionally substituted alkylheteroarylene, optionally substituted C₁-C₃ alkylene, optionally substituted heteroalkylene, optionally substituted cycloalkylene, optionally substituted heterocycloalkylene and optionally substituted —(CH₂)ₘ—C(O)—N(R⁴) (CH₂)ₙ—, wherein n is an integer from 0 to 6, m is an integer from 0 to 6;

M is selected from the group consisting of O, S, NH, NR⁴, NOH and NOR⁴;

R² is selected from the group consisting of H, C₁-C₁₀ alkyl, alkenyl, heteroalkyl, haloalkyl, alkynyl, aryl, cycloalkyl, heterocycloalkyl, heteroaryl, C₄-C₈ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylosulfonyl, arylsulfonyl, aminosulfonyl, —C(O)OR⁴, —CONHR⁴, —NHCONHR⁴, C(═NOH)R⁴, and acyl;

R³ is selected from the group consisting of H, C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylosulfonyl, arylsulfonyl, aminosulfonyl, —C(O)OR⁴, —CONHR⁴, —NHCONHR⁴, C(═NOH)R⁴, and acyl;

Q is selected from the group consisting of —S(O)₂—, —CO— and —C(═S)—;

G is selected from optionally substituted aryl, optionally substituted heteroaryl, alkyl, optionally substituted cycloalkyl, optionally substituted heterocycloalkyl, optionally substituted arylalkyl and optionally substituted heteroarylalkyl, wherein the substituents are independently selected from the group consisting of X, Y, R⁴, hydroxyl, hydroxyalkyl, alkoxy, amino, alkylamino, aminoalkyl, acylamino, phenoxy, alkoxyalkyl, benzyloxy, alkylosulfonyl, arylsulfonyl, aminosulfonyl, —C(O)OR⁴, —C(O)OH, —SH, —CONHR⁴, —NHCONHR⁴, and C(═NOH)R⁴;

R⁴ is selected from the group consisting of C₁-C₄ alkyl, heteroalkyl, aryl, heteroaryl and acyl;

X and Y are the same or different and are independently selected from the group consisting of H, halo, C₁-C₄ alkyl, NO₂, OR⁴, SR⁴, C(O)R⁵, and NR⁶R⁷;

R⁵ is C₁-C₄ alkyl;

R⁶ and R⁷ are the same or different and are independently selected from the group consisting of H, C₁-C₆ alkyl, C₄-C₈ cycloalkyl, C₄-C₉ heterocycloalkyl, aryl, heteroaryl, arylalkyl and heteroaryl alkyl.

or a pharmaceutically acceptable salt or prodrug thereof.

Particularly preferred compounds of Formula (2) are those of Formula (2b) and (2c).

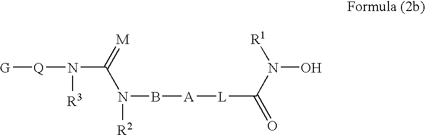

or a pharmaceutically acceptable salt or prodrug thereof.

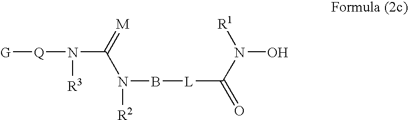

or a pharmaceutically acceptable salt or prodrug thereof.

Another preferred subset of compounds are those of Formula (2d) wherein R¹═R³═H; R², X, Y, Z, A, B, R³ and R⁴ are the same as for Formula (2).

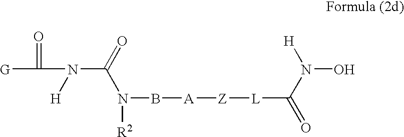

or a pharmaceutically acceptable salt or prodrug thereof.

In further embodiments there is disclosed a compound of Formula (2e) wherein R¹═R³═H; R², X, Y, Z, A, B, R³ and R⁴ are the same as for Formula (2).

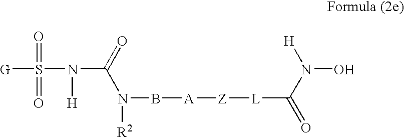

or a pharmaceutically acceptable salt or prodrug thereof.

In further embodiments there is disclosed a compound of Formula (2f) wherein R², X, Y, L, A, B, G, R³ and R⁴ are the same as for Formula (2).

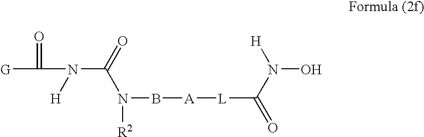

or a pharmaceutically acceptable salt or prodrug thereof.

In further embodiments there is disclosed a compound of Formula (2g) wherein R², X, Y, L, B, G and R⁴ are the same as for Formula (2).

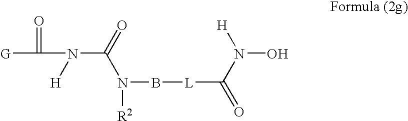

or a pharmaceutically acceptable salt or prodrug thereof.

In a further embodiment there is disclosed a compound of Formula (2h)

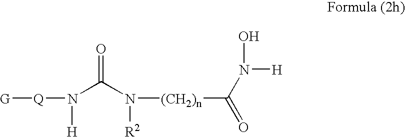

wherein
n=integer from 1 to 8,

### Q=—C(O)— or —SO₂—, G and R² are as for Formula (I),

or a pharmaceutically acceptable salt or prodrug thereof.

In a further embodiment there is provided a compound of Formula (2i)

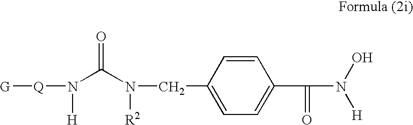

wherein

### Q=—C(O)— or —SO₂—, and

### G and R² are as for Formula (I),

or a pharmaceutically acceptable salt or prodrug thereof.

In a further embodiment there is provided a compound of Formula (2j)

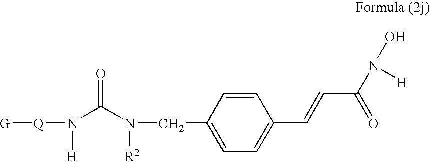

wherein

### Q=—C(O)— or —SO₂—, and

### G and R² are as for Formula (I),

or a pharmaceutically acceptable salt or prodrug thereof.

There is also provided a compound of Formula (2k)

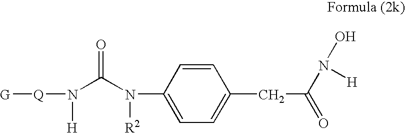

wherein

### Q=—C(O)— or —SO₂—, and

### G and R² are as for Formula (I),

or a pharmaceutically acceptable salt or prodrug thereof.

There are also provided compounds of Formula (21)

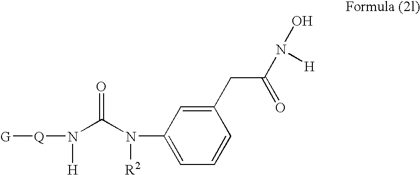

wherein

### Q=—C(O)— or —SO₂—, and

### G and R² are as for Formula (I),

or a pharmaceutically acceptable salt or prodrug thereof.

In a further embodiment there are provided compounds of Formula (2m)

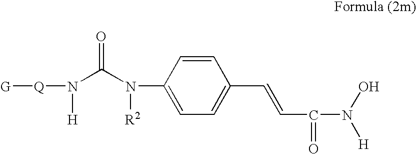

wherein

### Q=—C(O)— or —SO₂—, and

### G and R² are as for Formula (I), or a pharmaceutically acceptable salt or prodrug thereof.

In further embodiments there is disclosed a compound of Formula (2n) wherein B is a single bond or CH₂, L is a single bond or selected from CH₂, CH₂CH₂, —CH═CH—, —C-triple bond-C—. B is attached to meta or para position of phenylene relative to L and G is selected from aryl, heteroaryl, alkyl and alkoxyalkyl.

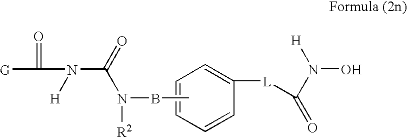

or a pharmaceutically acceptable salt or prodrug thereof.

In further embodiments there is disclosed a compound of Formula (2p) wherein n is an integer from 1 to 8; G is selected from aryl, heteroaryl, alkyl and heteroalkyl. R² is selected from H, alkyl, heteroalkyl, cycloalkyl, heterocycloalkyl, aryl, arylalkyl, arylheteroalkyl, heteroarylalkyl, and heteroarylheteroalkyl.

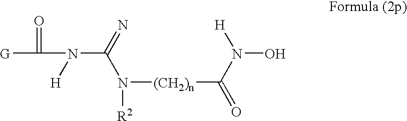

or a pharmaceutically acceptable salt or prodrug thereof.

In further embodiments there is disclosed a compound of Formula (2q) wherein B is a single bond or CH₂, L is a single bond or selected from CH₂, CH₂CH₂, —CH—CH—, —C-triple bond-C—, R² is selected from H, alkyl, heteroalkyl, cycloalkyl, heterocycloalkyl, aryl, alkylaryl, heteroalkyl, heteroarylalkyl, heteroarylheteroalkyl. X is selected from H, halo, C₁-C₄ alkyl, alkoxy, alkylamino; B is attached to meta or para position of phenylene relative to L.

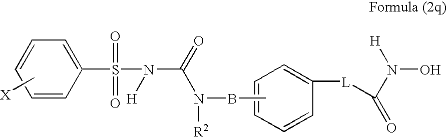

or a pharmaceutically acceptable salt or prodrug thereof.

In further embodiments there is disclosed a compound of (2r) wherein n is an integer from 1 to 8, X is selected from H, halo, C₁-C₄ alkyl, alkoxy, alkylamino.

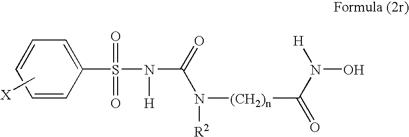

or a pharmaceutically acceptable salt or prodrug thereof.

As with any group of structurally related compounds which possess a particular utility, certain groups are preferred for the compounds of the Formula (I), (2), (2a), (2b), (2c), (2d), (2e), (2f), (2g), (2h), (21), (2j), (2k), (2l), (2m), (2n), (2p), (2q) and (2r) in their end use application.

In those embodiments in which it is present R¹ is preferably H or C₁-C₄ alkyl, more preferably H or methyl, most preferably H.

M is preferably O or S, most preferably O.

Q is preferably S(O)₂ or CO, most preferably CO.

G is preferably optionally substituted aryl, more preferably optionally substituted phenyl, most preferably 4-methyl phenyl or phenyl.

R² is preferably selected from the group consisting of H, optionally substituted alkyl, optionally substituted heteroalkyl, optionally substituted cycloalkyl, optionally substituted heterocycloalkyl, optionally substituted aryl, optionally substituted heteroaryl, optionally substituted arylalkyl, optionally substituted arylheteroalkyl, optionally substituted heteroarylalkyl, optionally substituted heteroarylheteroalkyl, optionally substituted cycloalkylalkyl and optionally substituted heterocycloalkylalkyl.

In a particularly preferred embodiment R² is selected from the group consisting of H, 2-(1H-indol-3-yl)-ethyl, 2-(2-methyl-1H-indol-3-yl)-ethyl, pyridin-3-ylmethyl, 3-hydroxy-propyl, 2-pyridin-2-yl-ethyl, 2-pyridin-3-yl-ethyl, pyridin-3-ylmethyl, 2-pyridin-4-yl-ethyl, benzyl, 3-phenyl-propyl, 2-phenoxy-ethyl, morpholin-4-yl, pyridin-2-yl, phenethyl, 2-(4-bromo-phenyl)-ethyl, 2-(4-fluoro-phenyl)-ethyl, 3-imidazol-1-yl-propyl, 2-(1H-imidazol-4-yl)-ethyl, 1H-Benzoimidazol-2-ylmethyl, 2-piperidin-1-yl-ethyl, 2-pyrrolidin-1-yl-ethyl, 2-cyclohex-1-enyl-ethyl, 2-ethyl-hexyl, 2-thiophen-2-yl-ethyl, 3,3-diphenyl-propyl, 2-biphenyl-4-yl-ethyl, -(4-phenoxy-phenyl, 2-(3-phenoxy-phenyl)-ethyl, 2-(2,3-dimethoxy-phenyl, 2-(2,4-dichloro-phenyl)-ethyl, cyclohexylmethyl, hexyl, isobutyl, 3-isopropoxy-propyl, 2-phenoxy-ethyl, 2-isopropoxy-ethyl, 3-methoxy-benzyl, 4-[1,2,3]thiadiazol-4-yl-benzyl, 2,4-dichloro-benzyl, 2-(2-methoxy-phenyl)-ethyl, 2-(3-fluoro-phenyl)-ethyl, 2-(2-fluoro-phenyl)-ethyl, 2,2-diphenyl-ethyl, 2-(4-methoxy-phenyl)-ethyl, 2-(3-chloro-phenyl)-ethyl, 4-phenyl-butyl, 3-phenyl-propyl, 3,3-diphenyl-propyl, 3-(4-methyl-piperazin-1-yl, 3-morpholin-4-yl-propyl, 3-(2-oxo-pyrrolidin-1-yl)-propyl, 3-pyrrolidin-1-yl-propyl, tetrahydro-furan-2-ylmethyl, 1,5-dimethyl-hexyl, 2-diethylamino-ethyl and 2-dimethylamino-ethyl.

It is even more preferred that R² is selected from the group consisting of H, 2-(1H-indol-3-yl)-ethyl, 2-(2-methyl-1H-indol-3-yl)-ethyl, pyridin-3-ylmethyl, 3-hydroxy-propyl, 2-pyridin-2-yl-ethyl, 2-pyridin-3-yl-ethyl, pyridin-2-ylmethyl, pyridin-3-ylmethyl, 2-pyridin-4-yl-ethyl, benzyl, 3-phenyl-propyl, 2-phenoxy-ethyl, 2-morpholino ethyl, 2-phenyl ethyl, 2-(4-bromo-phenyl)-ethyl, 2-(4-fluoro-phenyl)-ethyl, 3-imidazol-1-yl-propyl, 2-(1H-imidazol-4-yl)-ethyl, 1H-Benzoimidazol-2-ylmethyl, 2-piperidin-1-yl-ethyl and 2-pyrrolidin-1-yl-ethyl.

In a most preferred embodiment R² is selected from the group consisting of H, 2-(1H-indol-3-yl)-ethyl, 2-(2-methyl-1H-indol-3-yl)-ethyl, 2-phenyl ethyl, 2-piperidin-1-yl-ethyl and 2-pyrrolidin-1-yl-ethyl.

It is preferred that R³ is H.

It is preferred that L is selected from the group consisting of a single bond, —CH₂—, —(CH₂)₂— and —CH═CH—. Accordingly in one preferred embodiment L is a bond. In another preferred embodiment L is a group of formula —CH₂—. In another preferred embodiment L is a group of formula —CH═CH—. The group of formula —CH═CH— is preferably in the “E” configuration.

Z is preferably a single bond.

A is preferably an optionally substituted arylene. In one preferred embodiment A is selected from the group consisting of 1,4-phenylene and 1,3-phenylene. It is particularly preferred that A is 1,4-phenylene.

It is preferred that B is selected from the group consisting of a single bond, methylene, ethylene, propylene, alkylarylene, and heteroalkylene. In one preferred embodiment B is methylene. In another preferred embodiment B is a bond. In another preferred embodiment B is ethylene. In yet another preferred embodiment B is propylene.

In one preferred embodiment the identities of B, A, Z and L are such that the group BAZL is a group of formula —CH₂)ₙ— wherein n is an integer from 1 to 7.

In another preferred embodiment the identities of B, A, Z are such that the group BAZ is a group of formula —(CH₂)— phenyl-

In a particularly preferred embodiment the identities of B, A, Z and L are such that the group BAZL is selected from the group consisting of

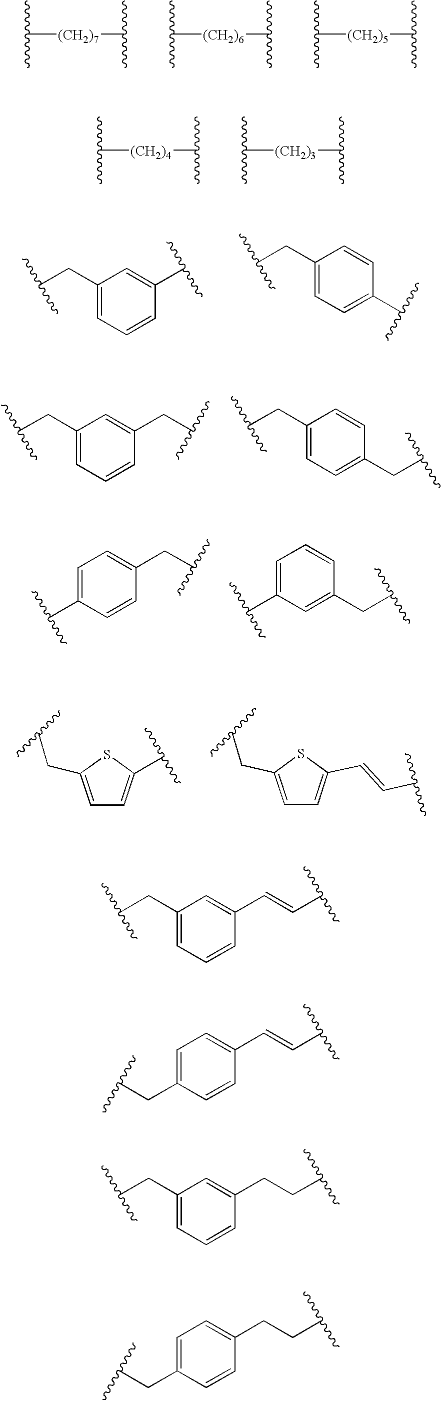

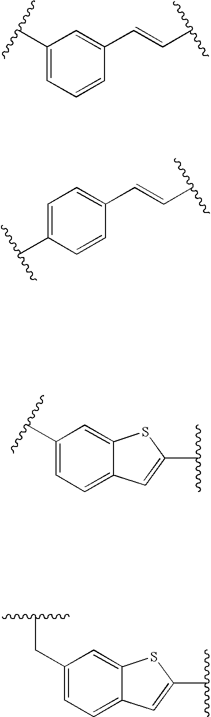

[symbol] is a single bond

In many of the formulae given herein the substituents are stated to be optionally substituted. Where substituents are present it is preferred that the optional substituents are selected from the group consisting of halogen, ═O, ═S, —CN, —NO₂, —CF₃, —OCF₃; alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkoxy, alkoxyalkyl, alkoxycycloalkyl, alkoxyheterocycloalkyl, alkoxyaryl, alkoxyheteroaryl, alkoxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, arylalkyl, heteroarylalkyl, cycloalkylalkyl, heterocycloalkylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —COOH, —COR⁵, —C(O)OR⁵, CONHR⁵, NHCOR⁵, NHCOOR⁵, NHCONHR⁵, C(═NOH)R⁵, —SH, —SR⁵, —OR⁵ and acyl, or

wherein each R⁵ is independently selected from the group consisting of alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted;

In addition to compounds of the invention as described above the embodiments disclosed are also directed to pharmaceutically acceptable salts, pharmaceutically acceptable prodrugs, and pharmaceutically active metabolites of such compounds, and pharmaceutically acceptable salts of such metabolites. Such compounds, salts, prodrugs and metabolites are at times collectively referred to herein as “HDAC inhibiting agents” or “HDAC inhibitors”. In certain embodiments the compounds disclosed are used to modify deacetylase activity, in some cases histone deacetylase activity and in some cases HDAC 8, or HDAC 1 activity.

The embodiments disclosed also relate to pharmaceutical compositions each comprising a therapeutically effective amount of a HDAC inhibiting agent of the embodiments described with a pharmaceutically acceptable carrier or diluent for treating cellular proliferative ailments. The term “effective amount” as used herein indicates an amount necessary to administer to a host to achieve a therapeutic result, e.g., inhibition of proliferation of malignant cancer cells, benign tumor cells or other proliferative cells.

The invention also relates to pharmaceutical compositions including a compound of the invention with a pharmaceutically acceptable carrier, diluent or excipient.

In yet a further aspect the present invention provides a method of treatment of a disorder caused by, associated with or accompanied by disruptions of cell proliferation and/or angiogenesis including administration of a therapeutically effective amount of a compound of Formula (I).

The method preferably involves administration of a compound of Formula (2) more preferably a compound of Formula (2a) or (2b) or (2c), most preferably a compound of (2e) to (2r).

The disorder is preferably selected from the group consisting of but not limited to cancer (e.g. breast cancer, colon cancer, prostate cancer, pancreatic cancer, leukemias, lymphomas), inflammatory diseases/immune system disorders, angiofibroma, cardiovascular diseases (e.g. restenosis, arteriosclerosis), fibrotic diseases (e.g. liver fibrosis), diabetes, autoimmune diseases, chronic and acute neurodegenerative disease like disruptions of nerval tissue, Huntington's disease and infectious diseases like fungal, bacterial and viral infections. In another embodiment the disorder is a proliferative disorder. The proliferative disorder is preferably cancer. The cancer can include solid tumors or hematologic malignancies.

The invention also provides agents for the treatment of a disorder caused by, associated with or accompanied by disruptions of cell proliferation and/or angiogenesis including a compound of Formula (I) as disclosed herein. The agent is preferably an anti-cancer agent.

The agent preferably contains a compound of Formula (2) more preferably a compound of Formula (2a) or (2b) or (2c), most preferably a compound of (2e) to (2r).

The invention also relates to the use of compounds of Formula (I) in the preparation of a medicament for the treatment of a disorder caused by, associated with or accompanied by disruptions of cell proliferation and/or angiogenesis. The disorder is preferably a proliferative disorder, most preferably a cancer.

The compounds of the present invention surprisingly show low toxicity, together with a potent anti-proliferative activity.

In yet a further embodiment the invention provides a method of treatment of a disorder, disease or condition that can be treated by the inhibition of histone deacetylase including administration of a therapeutically effective amount of a compound of Formula (I).

In yet a further embodiment the invention provides a method of treatment of a disorder, disease or condition that are mediated by deacetylase activity such as histone deacetylase including administration of a therapeutically effective amount of a compound of Formula (I).

The method preferably includes administration of a compound of Formula (2), more preferably a compound of Formula (2a) or (2b) or (2c), most preferably a compound of (2e) to (2r) as described herein.

The disorder is preferably selected from the group consisting of but not limited to Proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, Intracerebral haemorrhage Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Interstitial keratitis, Diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, inflammatory bowel disease, Colitis Ulcerosa, Alcoholic hepatitis, Diabetes, Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, depression and dementia; Cardiovascular Diseases including Heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.

The invention also provides agents for the treatment of a disorder, disease or condition that can be treated by the inhibition of histone deacetylase including a compound of Formula (I) as disclosed herein. The agent is preferably an anti-cancer agent.

The invention also relates to the use of compounds of Formula (I) in the preparation of a medicament for the treatment of a disorder, disease or condition that can be treated by the inhibition of histone deacetylase.

In another embodiment the invention provides a method of modifying deacetylase activity including contacting the deacetylase with a compound of Formula (I). The deacetylase activity is preferably histone deacetylase activity, even more preferably class I histone deacetylase activity. The histone deacetylase is preferably HDAC1 or HDAC8.

The invention also provides a method for inhibiting cell proliferation including administration of an effective amount of a compound according to Formula (I).

In yet an even further aspect the invention provides a method of treatment of a neurodegenerative disorder in a patient including administration of a therapeutically effective amount of a compound of Formula (I). The method preferably includes administration of a compound of Formula (2) more preferably a compound of Formula (2a) or (2b) or (2c), most preferably a compound of (2e) to (2r) as described herein. The neurodegenerative disorder is preferably Huntington's Disease.

The invention also provides agents for the treatment of neurodegenerative disorder including a compound of Formula (I) as disclosed herein. The agent is preferably anti-Huntington's disease agent.

The invention also relates to the use of compounds of Formula (I) in the preparation of a medicament for the treatment of a neurodegenerative disorder. The neurodegenerative disorder is preferably Huntington's Disease.

In yet an even further aspect the invention provides a method of treatment of an inflammatory disease and/or immune system disorder in a patient including administration of a therapeutically effective amount of a compound of Formula (I). The method preferably includes administration of a compound of Formula (2) more preferably a compound of Formula (2a) or (2b) or (2c), most preferably a compound of (2e) to (2r) as described herein. In one embodiment the inflammatory disease and/or immune system disorder is rheumatoid arthritis. In another embodiment the inflammatory disease and/or immune system disorder is Systemic Lupus Erythematosus.

The invention also provides agents for the treatment of inflammatory disease and/or immune system disorder including a compound of Formula (I) as disclosed herein.

The invention also relates to the use of compounds of Formula (I) in the preparation of a medicament for the treatment of inflammatory disease and/or immune system disorder. In one embodiment the inflammatory disease and/or immune system disorder is rheumatoid arthritis. In another embodiment the inflammatory disease and/or immune system disorder is Systemic Lupus Erythematosus.

In another embodiment the present invention provides the use of a compound of Formula (I) to modify deacetylase activity, preferably histone deacetylase activity, even more preferably HDAC1 or HDAC8.

The invention also provides the use of a compound of Formula (I) to treat cancer. In another embodiment, the cancer is selected from a group including but not limited to breast cancer, lung cancer, ovarian cancer, prostate cancer, head and neck cancer, renal cancer, gastric cancer, colon cancer, pancreatic cancer and brain cancer.

The present invention also provides the use of a compound of Formula (I) in the preparation of a medicament for the treatment of hematologic malignancies. The hematologic malignancy is preferably selected from the group consisting of B-cell lymphoma, T-cell lymphoma and leukemia.

The invention also provides a method for the treatment of a hematologic malignancy including administration of an effective amount of a compound of Formula (I).

The invention also provides an agent for the treatment of hematologic malignancy including a compound of Formula (I).

The invention also provides the use of a compound of Formula (I) in the preparation of a medicament for the treatment of solid tumors. The solid tumor is preferably selected from the group consisting of breast cancer, lung cancer, ovarian cancer, prostate cancer, head and neck cancer, renal cancer, gastric cancer, colon cancer, pancreatic cancer and brain cancer.

The invention also provides a method of treatment of a solid tumor including administration of an effective amount of a compound of Formula (I). The solid tumor is preferably selected from the group consisting of breast cancer, lung cancer, pancreatic cancer, ovarian cancer, prostate cancer, head and neck cancer, renal cancer, gastric cancer, colon cancer and brain cancer.

The invention also provides agents for the treatment of solid tumors including a compound of Formula (I).

In yet a further aspect the invention provides for the use of a compound of Formula (I) in the preparation of a medicament for the induction of cell death such as apoptosis of tumor cell.

The invention also provides a method of inhibiting tumor cell proliferation including the administration of a compound according to Formula (I).

The invention also provides a method of inhibiting the activity of histone deacetylase including contacting the histone deacetylase with an effective amount of a compound according to Formula (I).

The invention also provides the use of a compound of Formula (I) in the manufacture of medicaments for the induction of apoptosis of tumor cells.

In an even further embodiment the invention provides a method of inducing apoptosis in tumor cells including administration of an effective amount of a compound of Formula (I).

### DETAILED DESCRIPTION OF THE INVENTION

There are disclosed hydroxamate compounds, for example acylurea/sulfonylurea containing hydroxamic acid in one of the substituents, that may be inhibitors of deacetylases, including but not limited to inhibitors of histone deacetylases. The hydroxamate compounds may be suitable for prevention or treatment of a disorder caused by, associated with or accompanied by disruptions of cell proliferation and/or angiogenesis when used either alone or together with a pharmaceutically acceptable carrier, diluent or excipient. An example of such a disorder is cancer.

As used herein the term ‘cancer’ is a general term intended to encompass the vast number of conditions that are characterised by uncontrolled abnormal growth of cells.

It is anticipated that the compounds of the invention will be useful in treating various cancers including but not limited to bone cancers including Ewing's sarcoma, osteosarcoma, chondrosarcoma and the like, brain and CNS tumors including acoustic neuroma, neuroblastomas, glioma and other brain tumors, spinal cord tumors, breast cancers, colorectal cancers, colon cancer, advanced colorectal adenocarcinomas, endocrine cancers including adenocortical carcinoma, pancreatic cancer, pituitary cancer, thyroid cancer, parathyroid cancer, thymus cancer, multiple endocrine neoplasma, gastrointestinal cancers including stomach cancer, esophageal cancer, small intestine cancer, Liver cancer, extra hepatic bile duct cancer, gastrointestinal carcinoid tumor, gall bladder cancer, genitourinary cancers including testicular cancer, penile cancer, prostate cancer, gynaecological cancers including cervical cancer, ovarian cancer, vaginal cancer, uterus/endometrium cancer, vulva cancer, gestational trophoblastic cancer, fallopian tube cancer, uterine sarcoma, head and neck cancers including oral cavity cancer, lip cancer, salivary gland cancer, larynx cancer, hypopharynx cancer, orthopharynx cancer, nasal cancer, paranasal cancer, nasopharynx cancer, leukemias including childhood leukemia, acute lymphocytic leukemia, acute myeloid leukemia, chronic lymphocytic leukemia, chronic myeloid leukemia, hairy cell leukemia, acute promyelocytic leukemia, plasma cell leukemia, myelomas, haematological disorders including myelodysplastic syndromes, myeloproliferative disorders, aplastic anemia, Fanconi anemia, Waldenstroms Macroglobulinemia, lung cancers including small cell lung cancer, non-small cell lung cancer, lymphomas including Hodgkin's disease, non-Hodgkin's lymphoma, cutaneous T-cell lymphoma, peripheral T-cell lymphoma, AIDS related Lymphoma, B-cell lymphoma, Burkitt's lymphoma, eye cancers including retinoblastoma, intraocular melanoma, skin cancers including melanoma, non-melanoma skin cancer, merkel cell cancer, soft tissue sarcomas such as childhood soft tissue sarcoma, adult soft tissue sarcoma, Kaposi's sarcoma, urinary system cancers including kidney cancer, Wilms tumor, bladder cancer, urethral cancer, and transitional cell cancer.

Preferred cancers that may be treated by the compounds of the present invention include but are not limited to breast cancer, colon cancer, pancreatic cancer, lung cancer, ovarian cancer, prostate cancer, head and neck cancer, renal cancer, gastric cancer and brain cancer.

Preferred cancers that may be treated by compounds of the present invention include but are not limited to B-cell lymphoma (e.g. Burkitt's lymphoma), leukemias (e.g. Acute promyelocytic leukemia), cutaneous T-cell lymphoma (CTCL) and peripheral T-cell lymphoma.

Preferred cancers that may be treated by compounds of the present invention include but are not limited to solid tumors and hematologic malignancies.

The compounds may also be used in the treatment of a disorder involving, relating to, or associated with dysregulation of histone deacetylase (HDAC).

There are a number of disorders that have been implicated by or known to be mediated at least in part by HDAC activity, where HDAC activity is known to play a role in triggering disease onset, or whose symptoms are known or have been shown to be alleviated by HDAC inhibitors. Disorders of this type that would be expected to be amenable to treatment with the compounds of the invention include the following but not limited to: Proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, intracerebreal haemorrphage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Intersititial keratitis, Diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, inflammatory bowel disease Colitis Ulcerosa, Alcoholic hepatitis, Diabetes, Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, mainia, depression and dementia; Cardiovascular Diseases including heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.

The hydroxamate compounds of the present invention have the following structure (I):

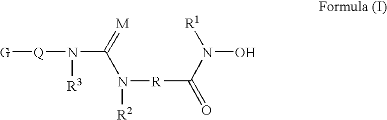

wherein

R is a linking moiety;

R¹ is selected from the group consisting of H, C₁-C₆ alkyl and acyl;

M is selected from the group consisting of O, S, NH, NR⁴, NOH and NOR⁴;

R² is selected from the group consisting of H, halogen, alkyl, alkenyl, alkynyl, haloalkyl, haloalkenyl, heteroalkyl, cycloalkyl, cycloalkenyl, heterocycloalkyl; heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴, NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted; or

R² together with the nitrogen to which it is attached and a portion of R form an optionally substituted heterocycloalkyl group;

R³ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴. NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted;

Q is selected from the group consisting of —S(O)₂—, —C(═O)— and —C(═S)—;

G is selected from the group consisting of optionally substituted alkyl, optionally substituted cycloalkyl, optionally substituted aryl, optionally substituted heteroaryl, optionally substituted heterocycloalkyl, optionally substituted arylalkyl, and optionally substituted heteroarylalkyl;

each R⁴ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted;

or a pharmaceutically acceptable salt or prodrug thereof.

In one preferred embodiment the compounds having the Formula (2)

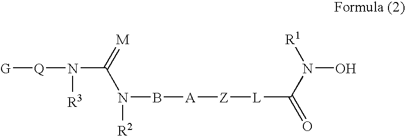

wherein

R¹ is selected from the group consisting of H, C₁-C₆ alkyl and acyl;

L is a single bond or is a C₁-C₅ hydrocarbon chain which may contain 0 to 2 multiple bonds independently selected from double bonds and triple bonds and wherein, the chain may optionally be interrupted by at least one of —O—, —S—, —S(O)— and —S(O)₂— and the chain may optionally be substituted with one or more substituents independently selected from the group consisting of C₁-C₄ alkyl;

Z is selected from the group consisting of a single bond, N(R¹), O, S, S(O) and S(O)₂;

A is selected from the group consisting of a single bond, optionally substituted arylene, optionally substituted heteroarylene, optionally substituted cycloalkylene and optionally substituted heterocycloalkylene;

B is selected from the group consisting of a single bond, optionally substituted acylamino, optionally substituted aminoacyl, optionally substituted arylene, optionally substituted heteroarylene, optionally substituted arylalkylene, optionally substituted heteroarylalkylene, optionally substituted alkylarylene, optionally substituted alkylheteroarylene, optionally substituted C₁-C₃ alkylene, optionally substituted heteroalkylene, optionally substituted cycloalkylene, optionally substituted heterocycloalkylene and optionally substituted —(CH₂)ₘ—C(O)—N(R⁴)—(CH₂)ₙ—, wherein n is an integer from 0 to 6, m is an integer from 0 to 6;

M is selected from the group consisting of O, S, NH, NR⁴, NOH and NOR⁴;

R² is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted; or

R² together with the nitrogen to which it is attached and a portion of B form an optionally substituted heterocycloalkyl group;

R³ is independently selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, aryiheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOH, COOR⁴, SH, CONHR⁴, NHCOR⁴, NHCOOR⁴, NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted;

Q is selected from the group consisting of —S(O)₂—, —C(═O)— and —C(═S)—;

G is selected from the group consisting of optionally substituted aryl, optionally substituted heteroaryl, optionally substituted alkyl, optionally substituted cycloalkyl, optionally substituted heterocycloalkyl, optionally substituted arylalkyl and optionally substituted heteroarylalkyl;

each R⁴ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted;

or a pharmaceutically acceptable salt or prodrug thereof.

In another preferred embodiment of the compounds of Formula (2) are compounds of Formula (2a)

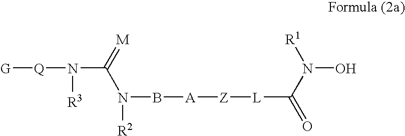

wherein

R¹ is selected from the group consisting of H, C₁-C₆ alkyl and acyl;

L is a single bond or is a C₁-C₅ hydrocarbon chain which may contain 0 to 2 multiple bonds independently selected from double bonds and triple bonds and wherein, the chain may optionally be interrupted by at least one of —O—, —S—, —S(O)— and —S(O)₂— and the chain may optionally be substituted with one or more substituents independently selected from the group consisting of C₁-C₄ alkyl;

Z is selected from the group consisting of a single bond, N(R¹), O, S, S(O) and S(O)₂;

A is selected from the group consisting of a single bond, optionally substituted arylene, optionally substituted heteroarylene, optionally substituted cycloalkylene and optionally substituted heterocycloalkylene;

B is selected from the group consisting of a single bond, optionally substituted acyl amino, optionally substituted aminoacyl, optionally substituted arylene, optionally substituted heteroarylene, optionally substituted arylalkylene, optionally substituted heteroarylalkylene, optionally substituted alkylarylene, optionally substituted alkylheteroarylene, optionally substituted C₁-C₃ alkylene, optionally substituted heteroalkylene, optionally substituted cycloalkylene, optionally substituted heterocycloalkylene and optionally substituted —(CH₂)ₘ—C(O)—N(R⁴)—(CH₂)ₙ—, wherein n is an integer from 0 to 6, m is an integer from 0 to 6;

M is selected from the group consisting of O, S, NH, NR⁴, NOH and NOR⁴;

R² is selected from the group consisting of H, C₁-C₁₀ alkyl, alkenyl, heteroalkyl, haloalkyl, alkynyl, aryl, cycloalkyl, heterocycloalkyl, heteroaryl, C₄-C₈ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, -phenoxy, alkoxyalkyl, benzyloxy, alkylosulfonyl, arylsulfonyl, aminosulfonyl, —C(O)OR⁴, —CONHR⁴, —NHCONHR⁴, C(═NOH)R⁴, and acyl;

R³ is selected from the group consisting of H, C₁-C₁₀ alkyl, alkenyl, heteroalkyl, haloalkyl, alkynyl, aryl, cycloalkyl, heterocycloalkyl, heteroaryl, C₄-C₈ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylosulfonyl, arylsulfonyl, aminosulfonyl, —C(O)OR⁴, —CONHR⁴, —NHCONHR⁴, C(═NOH)R⁴, and acyl;

Q is selected from the group consisting of —S(O)₂—, —CO— and —C(═S)—;

G is selected from optionally substituted aryl, optionally substituted heteroaryl, alkyl, optionally substituted cycloalkyl, optionally substituted heterocycloalkyl, optionally substituted arylalkyl and optionally substituted heteroarylalkyl, wherein the substituents are independently selected from the group consisting of X, Y, R⁴, hydroxyl, hydroxyalkyl, alkoxy, amino, alkylamino, aminoalkyl, acylamino, phenoxy, alkoxyalkyl, benzyloxy, alkylosulfonyl, arylsulfonyl, aminosulfonyl, —C(O)OR⁴, —C(O)OH, —SH, —CONHR⁴, —NHCONHR⁴, and C(═NOH)R⁴;

R⁴ is selected from the group consisting of C₁-C₄ alkyl, heteroalkyl, aryl, heteroaryl and acyl;

X and Y are the same or different and are independently selected from the group consisting of H, halo, C₁-C₄ alkyl, NO₂, OR⁴, SR⁴, C(O)R⁵, and NR⁶R⁷;

R⁵ is C₁-C₄ alkyl;

R⁶ and R⁷ are the same or different and are independently selected from the group consisting of H, C₁-C₆ alkyl, C₄-C₈ cycloalkyl, C₄-C₉ heterocycloalkyl, aryl, heteroaryl, arylalkyl and heteroaryl alkyl.

or a pharmaceutically acceptable salt or prodrug thereof.

Particularly preferred embodiments within the scope of these formulae are as described previously.

In particular embodiments the compound is selected from the group consisting of the following compounds in the table below

| | |
|---|---|
| 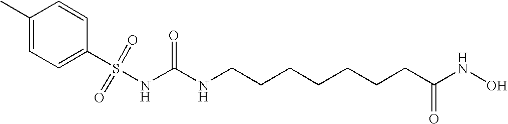 | 8-[3-(4-methylbenzenesulfonyl)-ureido])-octanoic acid hydroxyamide, |
| 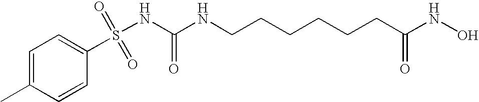 | 7-[3-(4-methylbenzenesulfonyl)-ureido])-heptanoic acid hydroxyamide, |
| 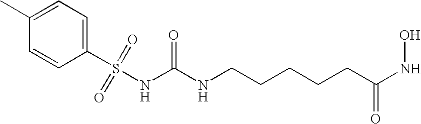 | 6-[3-(4-methylbenzenesulfonyl)-ureido])-hexanoic acid hydroxyamide, |
| 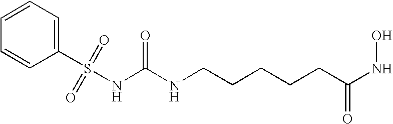 | 6-[3-(benzenesulfonyl)-ureido])-hexanoicacid hydroxyamide, |
| 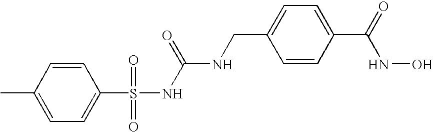 | N-Hydroxy-4-[3-(4-methylbenzenesulfonyl)ureido]methyl-benzamide, |
| 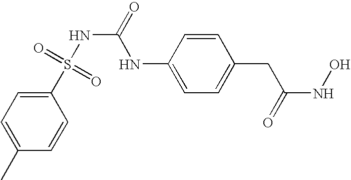 | N-Hydroxy-2-{4-[3-(4-methylbenzenesulfonyl)ureido]-phenyl}-acetamide, |
| 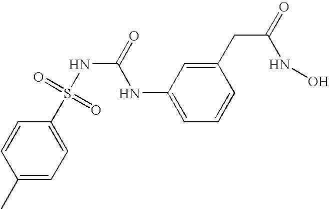 | N-Hydroxy-2-{3-[3-(4-methylbenzenesulfonyl)ureido]-phenyl}-acetamide, |
| 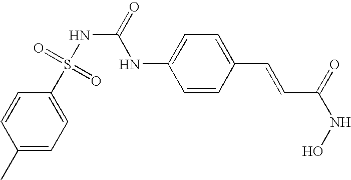 | N-Hydroxy-3-{4-[3-(4-methylbenzenesulfonyl)ureido]-phenyl}-acrylamide, |
| 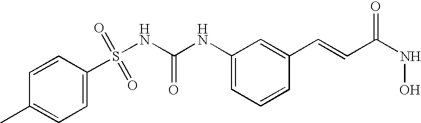 | N-Hydroxy-3-{3-[3-(4-methylbenzenesulfonyl)ureido]-phenyl}-acrylamide, |
| 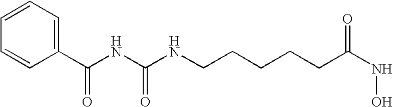 | 6-(3-Benzoyl-ureido)-hexanoic acidhydroxyamide, |
| 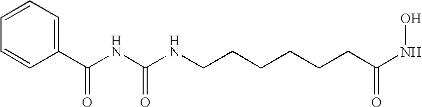 | 7-(3-Benzoyl-ureido)-heptanoic acidhydroxyamide, |
| 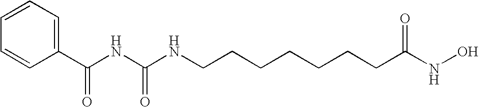 | 8-(3-Benzoyl-ureido)-octanoic acidhydroxyamide, |
| 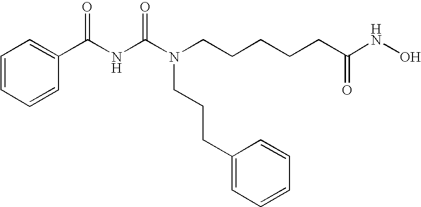 | 6-[3-Benzoyl-1-(3-phenyl-propyl)-ureido]-hexanoic acid hydroxyamide, |
| 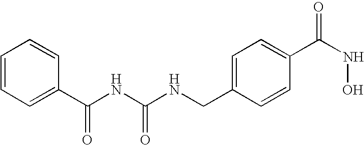 | 4-(3-Benzoyl-ureidomethyl)-N-hydroxy-benzamide, |
| 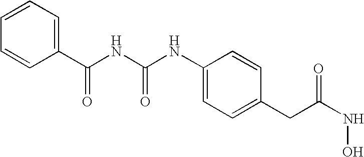 | 2-[4-(3-Benzoyl-ureido)-phenyl]-N-hydroxy-acetamide, |
| 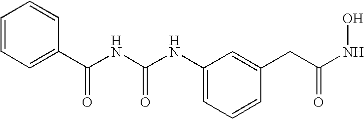 | 2-[3-(3-Benzoyl-ureido)-phenyl]-N-hydroxy-acetamide, |
| 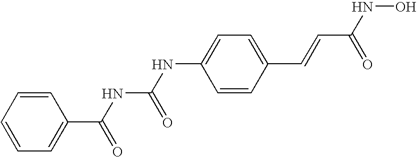 | 3-[4-(3-Benzoyl-ureido)-phenyl]-N-hydroxy-acrylamide, |
| 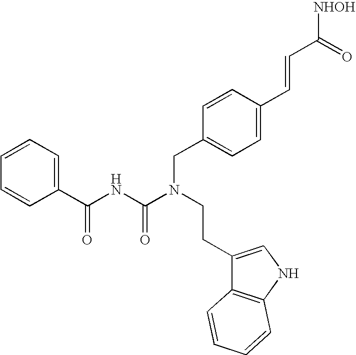 | 3-(4-{3-Benzoyl-1-[2-(1H-indol-3-yl)-ethyl]-ureidomethyl}-phenyl)-N-hydroxy-acrylamide, |
| 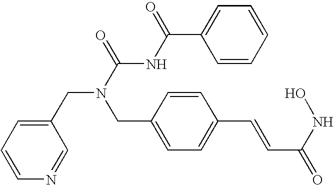 | 3-[4-(3-Benzoyl-1-pyridin-3-ylmethyl-ureidomethyl)-phenyl]-N-hydroxy-acrylamide, |
| 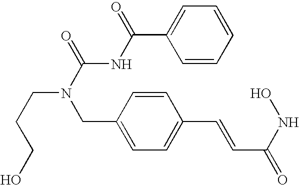 | 3-{4-[3-Benzoyl-1-(3-hydroxy-propyl)-ureidomethyl]-phenyl}-N-hydroxy-acrylamide, |
| 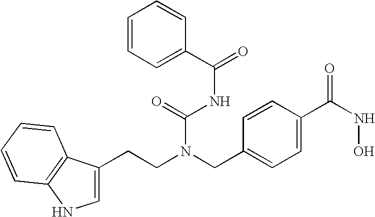 | 4-{3-Benzoyl-1-[2-(1H-indol-3-yl)-ethyl]-ureidomethyl}-N-hydroxy-benzamide, |
| 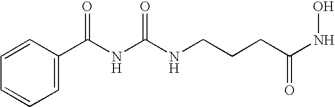 | 4-(3-Benzoyl-ureido)-N-hydroxy-butyramide, |
| 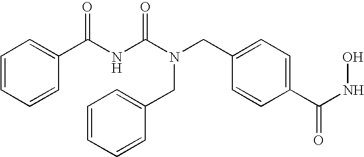 | 4-(3-Benzoyl-1-benzyl-ureidomethyl)-N-hydroxy-benzamide, |
| 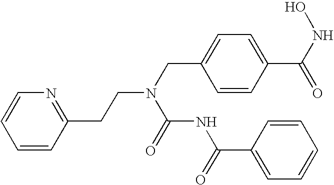 | 4-[3-Benzoyl-1-(2-pyridin-2-yl-ethyl)-ureidomethyl]-N-hydroxy-benzamide, |
| 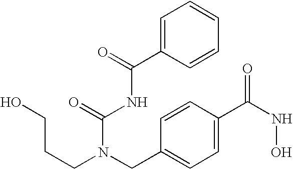 | 4-[3-Benzoyl-1-(3-hydroxy-propyl)-ureidomethyl]-N-hydroxy-benzamide, |
| 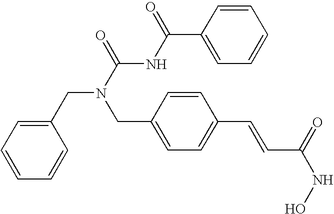 | 3-[4-(3-Benzoyl-1-benzyl-ureidomethyl)-phenyl]-N-hydroxy-acrylamide, |
| 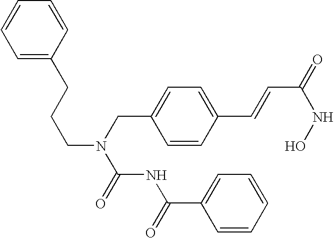 | 3-{4-[3-Benzoyl-1-(3-phenyl-propyl)-ureidomethyl]-phenyl}-N-hydroxy-acrylamide, |
| 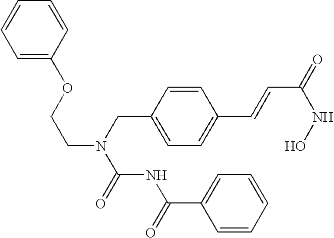 | 3-{4-[3-Benzoyl-1-(2-phenoxy-ethyl)-ureidomethyl]-phenyl}-N-hydroxy-acrylamide, |
| 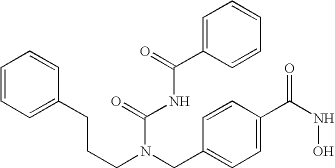 | 4-[3-Benzoyl-1-(3-phenyl-propyl)-ureidomethyl]-N-hydroxy-benzamide, |
| 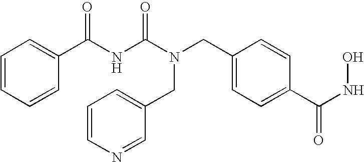 | 4-(3-Benzoyl-1-pyridin-3-ylmethyl-ureidomethyl)-N-hydroxy-benzamide, |
| 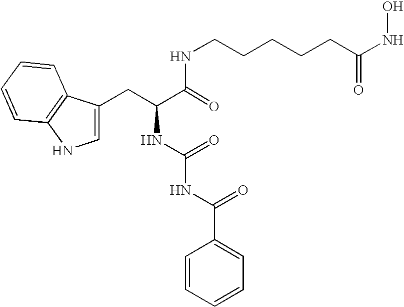 | (S)-6-[2-(3-Benzoyl-ureido)-3-(1H-indol-3-yl)-propionylamino]-hexanoic acidhydroxyamide, |
| 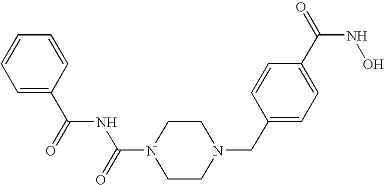 | 4-(4-Benzoylaminocarbonyl-piperazin-1-ylmethyl)-N-hydroxy-benzamide, |
| 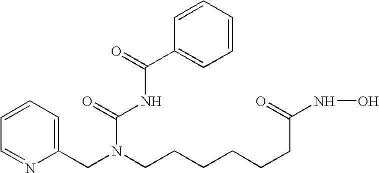 | 7-(3-Benzoyl-1-pyridin-2-ylmethyl-ureido)-heptanoic acid hydroxyamide, |
| 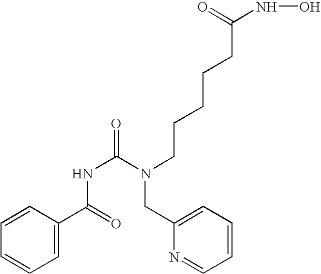 | 6-(3-Benzoyl-1-pyridin-2-ylmethyl-ureido)-hexanoic acid hydroxyamide, |
| 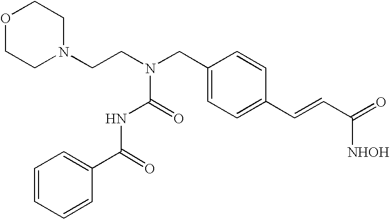 | 3-{4-[3-Benzoyl-1-(2-morpholin-4-yl-ethyl)-ureidomethyl]-phenyl}-N-hydroxy-acrylamide, |
| 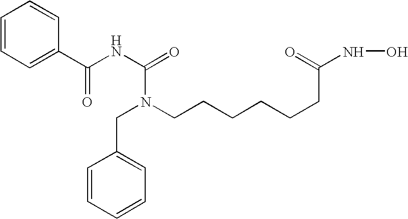 | 7-(3-Benzoyl-1-benzyl-ureido)-heptanoicacid hydroxyamide, |
| 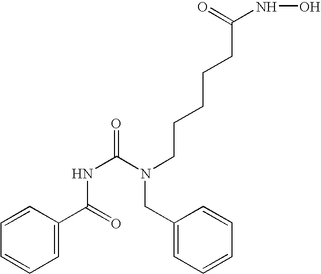 | 6-(3-Benzoyl-1-benzyl-ureido)-hexanoicacid hydroxyamide, |
| 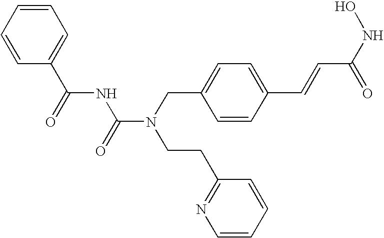 | 3-{4-[3-Benzoyl-1-(2-pyridin-2-yl-ethyl)-ureidomethyl]-phenyl}-N-hydroxy-acrylamide, |
| 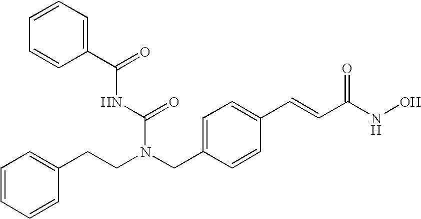 | 3-[4-(3-Benzoyl-1-phenethyl-ureidomethyl)-phenyl]-N-hydroxy-acrylamide, |
| 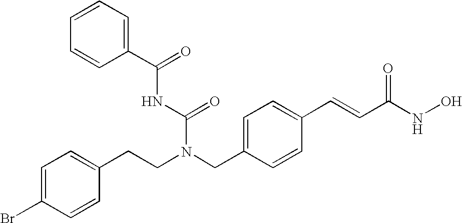 | 3-(4-{3-Benzoyl-1-[2-(4-bromo-phenyl)-ethyl]-ureidomethyl}-phenyl)-N-hydroxy-acrylamide, |
| 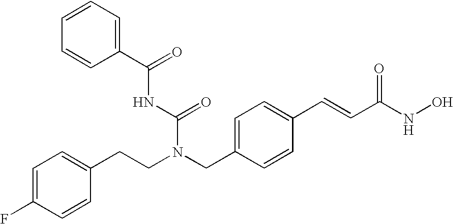 | 3-(4-{3-Benzoyl-1-[2-(4-fluoro-phenyl)-ethyl]-ureidomethyl}-phenyl)-N-hydroxy-acrylamide, |
| 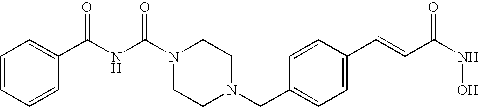 | N-{4-[4-(2-Hydroxycarbamoyl-vinyl)-benzyl]-piperazine-1-carbonyl}-benzamide, |
| 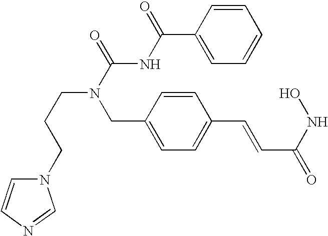 | 3-{4-[3-Benzoyl-1-(3-imidazol-1-yl-propyl)-ureidomethyl]phenyl}-N-hydroxy-acrylamide, |
| 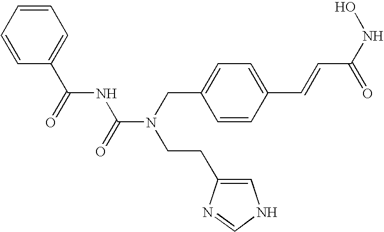 | 3-(4-{3-Benzoyl-1-[2-(1H-imidazol-4-yl)-ethyl]-ureidomethyl}-phenyl)-N-hydroxy-acrylamide, |
| 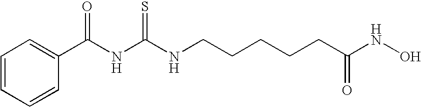 | 6-(3-Benzoyl-thioureido)-hexanoic acidhydroxyamide, |
| 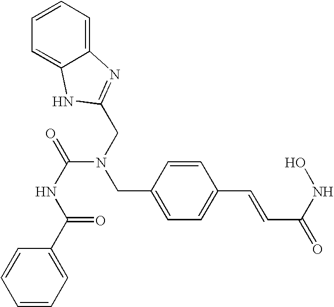 | 3-{4-[1-(1H-Benzoimidazol-2-ylmethyl)-3-benzoyl-ureidomethyl]-phenyl}-N-hydroxy-acrylamide, |
| 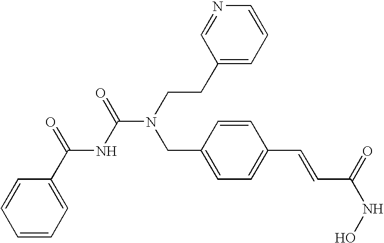 | 3-{4-[3-Benzoyl-1-(2-pyridin-3-yl-ethyl)-ureidomethyl]-phenyl}-N-hydroxy-acrylamide, |
| 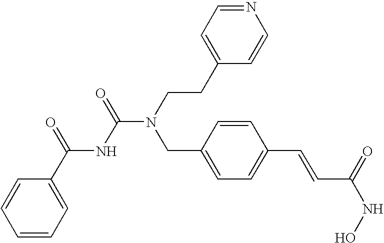 | 3-{4-[3-Benzoyl-1-(2-pyridin-4-yl-ethyl)-ureidomethyl]-phenyl}-N-hydroxy-acrylamide, |
| 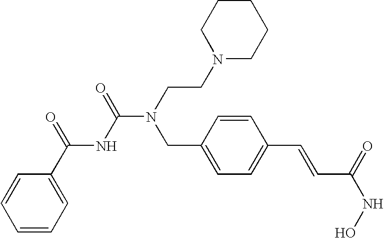 | 3-{4-[3-Benzoyl-1-(2-piperidin-1-yl-ethyl)-ureidomethyl]-phenyl}-N-hydroxy-acrylamide, |
| 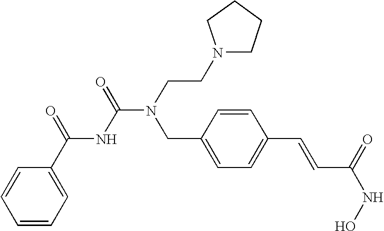 | 3-{4-[3-Benzoyl-1-(2-pyrrolidin-1-yl-ethyl)-ureidomethyl]-phenyl}-N-hydroxy-acrylamide, |

As used herein, the term unsubstituted means that there is no substituent or that the only substituents are hydrogen.

The term “optionally substituted” as used throughout the specification denotes that the group may or may not be further substituted or fused (so as to form a condensed polycyclic system), with one or more substituent groups. Preferably the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkoxy, alkoxyalkyl, alkoxycycloalkyl, alkoxyheterocycloalkyl, alkoxyaryl, alkoxyheteroaryl, alkoxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, arylalkyl, heteroarylalkyl, cycloalkylalkyl, heterocycloalkylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —COOH, —COR⁵, —C(O)OR⁵, CONHR⁵, NHCOR⁵, NHCOOR⁵, NHCONHR⁵, C(═NOH)R⁵, —SH, —SR⁵, —OR⁵ and acyl.

“Halogen” represents chlorine, fluorine, bromine or iodine.

“Alkyl” as a group or part of a group refers to a straight or branched aliphatic hydrocarbon group, preferably a C₁-C₁₄ alkyl, more preferably C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like.

“Alkylamino” includes both monoalkylamino and dialkylamino, unless specified. “Monoalkylamino” means a —NH-Alkyl group, “Dialkylamino” means a —N(alkyl)₂ group, in which the alkyl is as defined as above. The alkyl group is preferably a C₁-C₆ alkyl group.

“Arylamino” includes both mono-arylamino and di-arylamino unless specified. Mono-arylamino means a group of formula aryl NH—, di-arylamino means a group of formula (aryl₂) N— where aryl is as defined herein.

“Acyl” means a R—C(═O)— or G-C(═S)— group in which R is selected from aryl, heteroaryl, alkyl, cycloalkyl, heterocycloalkyl, arylalkyl and heteroarylalkyl as described herein. G could be further substituted. Examples of acyl include acetyl, benzoyl, phenylacetyl.

“Alkenyl” as group or part of a group denotes an aliphatic hydrocarbon group containing at least one carbon-carbon double bond and which may be straight or branched preferably having 2-14 carbon atoms, more preferably 2-12 carbon atoms, most preferably 26 carbon atoms, in the chain. The group may contain a plurality of double bonds in the normal chain and the orientation about each is independently E or Z. Exemplary alkenyl group include, but are not limited to, ethenyl and propenyl.

“Alkoxy” refers to an —O-alkyl group in which alkyl is defined herein. Preferably the alkoxy is a C₁-C₆ alkoxy. Examples include, but are not limited to, methoxy and ethoxy.

“Alkenyloxy” refers to an —O— alkenyl group in which alkenyl is as defined herein. Preferred alkenyloxy groups are C₁-C₆ alkenyloxy groups.

“Alkynyloxy” refers to an —O-alkynyl group in which alkynyl is as defined herein. Preferred alkynyloxy groups are C₁-C₆ alkynyloxy groups.

“Alkoxycarbonyl” refers to an —C(O)—O-alkyl group in which alkyl is as defined herein. The alkyl group is preferably a C₁-C₆ alkyl group. Examples include, but not limited to, methoxycarbonyl and ethoxycarbonyl.

“Alkylsulfinyl” means a —S(O)-alkyl group in which alkyl is as defined above. The alkyl group is preferably a C₁-C₆ alkyl group. Exemplary alkylsulfinyl groups include, but not limited to, methylsulfinyl and ethylsulfinyl.

“Alkylsulfonyl” refers to a —S(O)₂-alkyl group in which alkyl is as defined above. The alkyl group is preferably a C₁-C₆ alkyl group. Examples include, but not limited to methylsulfonyl and ethylsulfonyl.

“Alkynyl as a group or part of a group means an aliphatic hydrocarbon group containing a carbon-carbon trip bond and which may be straight or branched preferably having from 2-14 carbon atoms, more preferably 2-12 carbon atoms in the chain, preferably 2-6 carbon atoms in the chain. Exemplary structures include, but not limited to, ethynyl and propynyl.

“Alkylaminocarbonyl” refers to an alkylamino-carbonyl group in which alkylamino is as defined above.

“Aminoacyl” refers to the formula —C(O)—(CH₂)ₘ—(CH)(NR⁶R⁷)—(CH₂)—R⁶ wherein R⁶ and R⁷ are as defined above, m and n are integers selected from 0 to 6.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇cycloalkyl or C₅₋₇ cycloalkenyl group are fused together to form a cyclic structure, such as tetrahydronaphthyl, indenyl or indanyl. The aryl group may be substituted by one or more substituent groups. When the aryl ring is divalent it has been referred to as “arylene” in this application.

“Arylalkenyl” means an aryl-alkenyl-group in which the aryl and alkenyl are as previously described. Exemplary arylalkenyl groups include phenylallyl.

“Arylalkyl” means an aryl-alkyl-group in which the aryl and alkyl moieties are as previously described. Preferred arylalkyl groups contains a C₁₋₅ alkyl moiety. Exemplary arylalkyl groups include benzyl, phenethyl and naphthelenemethyl.

“Cycloalkyl” refers to a saturated or partially saturated, monocyclic or fused or spiro polycyclic, carbocycle preferably containing from 3 to 9 carbons per ring, such as cyclopropyl, cyclobutyl, cyclopentyl, cyclohexyl and the like, unless otherwise specified.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

“Cycloalkylalkyl” means a cycloalkyl-alkyl-group in which the cycloalkyl and alkyl moieties are as previously described. Exemplary monocycloalkylalkyl groups include cyclopropylmethyl, cyclopentylmethyl, cyclohexylmethyl and cylcoheptylmethyl.

“Heterocycloalkyl” refers to a ring containing from at least one heteroatom selected from nitrogen, sulfur, oxygen, preferably from 1 to 3 heteroatoms. Each ring is preferably from 3 to 4 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane.

“Heterocycloalkenyl” refers to a heterocycloalkyl as described above but containing at least one double bond.

“Heterocycloalkylalkyl” refers to a heterocycloalkyl-alkyl group in which the heterocycloalkyl and alkyl moieties are as previously described. Exemplary heterocycloalkylalkyl groups include (2-tetrahydrofuryl)methyl, (2-tetrahydrothiofuranyl)methyl.

“Heteroalkyl” refers to a straight- or branched-chain alkyl group preferably having from 2 to 14 carbons atoms, more preferably 2 to 10 carbon atoms in the chain, wherein one or more of the carbon atoms have been replaced by a heteroatom selected from S, O, and N. Exemplary heteroalkyls include alkyl ethers, secondary and tertiary alkyl amines, alkyl sulfides, and the like.

“Cycloalkenyl” means an optionally substituted non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups.

“Heteroaryl” refers to a monocyclic, or fused polycyclic, aromatic heterocycle (ring structure preferably having a 5 to 10 member aromatic ring containing one or more heteroatoms selected from N, O and S). Typical heteroaryl substituents include furyl, thienyl, pyrrole, pyrazole, triazole, thiazole, oxazole, pyridine, pyrimidine, isoxazolyl, pyrazine, indole, benzimidazole, and the like. When the heteroaryl ring is divalent it has been referred to as “heteroarylene” in this application.

“Heteroarylalkyl” means a heteroaryl-alkyl group in which the heteroaryl and alkyl moieties are as previously described. Preferred heteroarylalkyl groups contain a lower alkyl moiety. Exemplary heteroarylalkyl groups include pyridylmethyl.

“Lower alkyl” as a group means unless otherwise specified, an aliphatic hydrocarbon group which may be straight or branched having 1 to 6 carbon atoms in the chain, more preferably 1 to 4 carbons such as methyl, ethyl, propyl (n-propyl or isopropyl) or butyl (n-butyl, isobutyl or tertiary-butyl.

“Sulfonyl” means a R—SO₂— group in which the R is selected from aryl, heteroaryl, alkyl, cycloalkyl, heterocycloalkyl, arylalkyl and heteroarylalkyl as described herein. G could be further substituted. Examples of sulfonyl include methanesulfonyl, benzenesulfonyl, 4-methylbenzenesulfonyl, naphthalene-2-sulfonyl, and the like.

It is understood that included in the family of compounds of Formula I as well as in Formulae 2 to 2k are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the inventive compounds may exist as single stereoisomers, racemates, and/or mixtures of enantiomers and/or diastereomers. All such single stereoisomers, racemates and mixtures thereof are intended to be within the scope of the present invention subject matter described and claimed.

Additionally, Formula I is intended to cover, where applicable, solvated as well as unsolvated forms of the compounds. Thus, each formula includes compounds having the indicated structure, including the hydrated as well as the non-hydrated forms.

In addition to compounds of the Formula I, the HDAC inhibiting agents of the various embodiments include pharmaceutically acceptable salts, prodrugs, and active metabolites of such compounds, and pharmaceutically acceptable salts of such metabolites.

The term “Pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula I may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Suitable pharmaceutically acceptable base addition salts of compounds of Formula I include metallic salts made from lithium, sodium, potassium, magnesium, calcium, aluminium, and zinc, and organic salts made from organic bases such as choline, diethanolamine, morpholine. Other examples of organic salts are: ammonium salts, quaternary salts such as tetramethylammonium salt; amino acid addition salts such as salts with glycine and arginine. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

“Prodrug” means a compound which is convertible in vivo by metabolic means (e.g. by hydrolysis, reduction or oxidation) to a compound of Formula I. For example an ester prodrug of a compound of formula I containing a hydroxyl group may be convertible by hydrolysis in vivo to the parent molecule. Suitable esters of compounds of Formula (I) containing a hydroxyl group, are for example acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates. As another example an ester prodrug of a compound of formula I containing a carboxy group may be convertible by hydrolysis in vivo to the parent molecule. (Examples of ester prodrugs are those described by F. J. Leinweber, Drug Metab. Res., 18:379, 1987).

Possible HDAC inhibiting agents include those having an IC₅₀ value of 5 μM or less.

Administration of compounds within Formula I to humans can be by any of the accepted modes for enteral administration such as oral or rectal, or by parenteral administration such as subcutaneous, intramuscular, intravenous and intradermal routes. Injection can be bolus or via constant or intermittent infusion. The active compound is typically included in a pharmaceutically acceptable carrier or diluent and in an amount sufficient to deliver to the patient a therapeutically effective dose. In various embodiments the inhibitor compound may be selectively toxic or more toxic to rapidly proliferating cells, e.g. cancerous tumors, than to normal cells.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state. A therapeutically effective amount can be readily determined by a skilled practitioner by the use of conventional techniques and by observing results obtained in analogous circumstances. In determining the effective amount a number of factors are considered including the species of the patient, its size, age, general health, the specific disease involved, the degree or severity of the disease, the response of the individual patient, the particular compound administered, the mode of administration, the bioavailability of the compound, the dose regimen selected, the use of other medication and other relevant circumstances.

In using the compounds of the invention they can be administered in any form or mode which makes the compound bioavailable. One skilled in the art of preparing formulations can readily select the proper form and mode of administration depending upon the particular characteristics of the compound selected, the condition to be treated, the stage of the condition to be treated and other relevant circumstances. We refer the reader to Remingtons Pharmaceutical Sciences, 19^{th} edition, Mack Publishing Co. (1995) for further information.

The compounds of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. As such in a further embodiment the present invention provides a pharmaceutical composition including a compound of Formula (I) and a pharmaceutically acceptable carrier, diluent or excipient. The compositions are prepared in manners well known in the art.

The invention in other embodiments provides a pharmaceutical pack or kit comprising one or more containers filled with one or more of the ingredients of the pharmaceutical compositions of the invention. In such a pack or kit can be found a container having a unit dosage of the agent (s). The kits can include a composition comprising an effective agent either as concentrates (including lyophilized compositions), which can be diluted further prior to use or they can be provided at the concentration of use, where the vials may include one or more dosages. Conveniently, in the kits, single dosages can be provided in sterile vials so that the physician can employ the vials directly, where the vials will have the desired amount and concentration of agent(s). Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

The compounds of the invention may be used or administered in combination with one or more additional drug (s) that include chemotherapeutic drugs or HDAC inhibitor drugs and/or procedures (e.g. surgery, radiotherapy) for the treatment of the disorder/diseases mentioned. The components can be administered in the same formulation or in separate formulations. If administered in separate formulations the compounds of the invention may be administered sequentially or simultaneously with the other drug (s).

In addition to being able to be administered in combination with one or more additional drugs that include chemotherapeutic drugs or HDAC inhibitor drugs the compounds of the invention may be used in a combination therapy. When this is done the compounds are typically administered in combination with each other. Thus one or more of the compounds of the invention may be administered either simultaneously (as a combined preparation) or sequentially in order to achieve a desired effect. This is especially desirable where the therapeutic profile of each compound is different such that the combined effect of the two drugs provides an improved therapeutic result.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

If desired, and for more effective distribution, the compounds can be incorporated into slow release or targeted delivery systems such as polymer matrices, liposomes, and microspheres.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, patches, sprays, ointments and inhalants. The active compound is mixed under sterile conditions with a pharmaceutically acceptable carrier and any needed preservatives, buffers, or propellants which may be required.

A preferred dosage will be a range from about 0.01 to 300 mg per kilogram of body weight per day. A more preferred dosage will be in the range from 0.1 to 100 mg per kilogram of body weight per day, more preferably from 0.2 to 80 mg per kilogram of body weight per day, even more preferably 0.2 to 50 mg per kilogram of body weight per day. A suitable dose can be administered in multiple sub-doses per day.

As discussed above, the compounds of the embodiments disclosed inhibit histone deacetylases. The enzymatic activity of a histone deacetylase can be measured using known methodologies [Yoshida M. et al, J. Biol. Chem., 265, 17174 (1990), J. Taunton et al, Science 1996 272: 408]. In certain embodiments, the histone deacetylase inhibitor interacts with and/or reduces the activity of more than one histone deacetylase in the cell, which can either be from the same class of histone deacetylase or different class of histone deacetylase. In some other embodiments, the histone deacetylase inhibitor interacts and/or reduces the activity of predominantly one histone deacetylase, for example HDAC-1, HDAC-3 or HDAC-8 which belongs to Class I HDAC enzymes [De Ruijter A. J. M. et al, Biochem. J., 370, 737-749 (2003)]. Certain preferred histone deacetylase inhibitors are those that interact with, and/or reduce the activity of a histone deacetylase which is involved in tumorigenesis, and these compounds may be useful for treating proliferative diseases. Examples of such cell proliferative diseases or conditions include cancer (include any metastases), psoriasis, and smooth muscle cell proliferative disorders such as restenosis. The inventive compounds may be particularly useful for treating tumors such as breast cancer, colon cancer, lung cancer, ovarian cancer, prostate cancer, head and/or neck cancer, or renal, gastric, pancreatic cancer and brain cancer as well as hematologic malignancies such as lymphoma and leukemias. In addition, the inventive compounds may be useful for treating a proliferative disease that is refractory to the treatment with other chemotherapeutics; and for treating hyperproliferative condition such as leukemias, psoriasis and restenosis. In other embodiments, compounds in this invention can be used to treat pre-cancer conditions including myeloid dysplasia, endometrial dysplasia and cervical dysplasia.

Additionally compounds of the various embodiments disclosed herein may be useful for treating neurodegenerative diseases, and inflammatory diseases and/or immune system disorders.

The disorder is preferably selected from the group consisting of cancer, inflammatory diseases and/or immune system disorders (e.g. rheumatoid arthritis, systemic lupus erythematosus), angiofibroma, cardiovascular diseases, fibrotic diseases, diabetes, autoimmune diseases, chronic and acute neurodegenerative disease like Huntington's disease, Parkinson's disease, disruptions of nerval tissue and infectious diseases like fungal, bacterial and viral infections. In another embodiment the disorder is a proliferative disorder.

The histone deacetylase inhibitors of the invention have significant antiproliferative effects and promotes differentiation, for example, cell cycle arrest in the G1 or G2 phase, and induce apoptosis

### Synthesis of Deacetylase Inhibitors

The compounds of this invention may be prepared using the reaction routes and synthesis schemes as described below, employing the techniques available in the art using starting materials that are readily available. The preparation of particular embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g., by appropriately protecting interfering groups, by changing to other suitable reagents known in the art, or by making routine modifications of reaction conditions. A list of suitable protecting groups in organic synthesis can be found in T. W. Greene and P. G. M. Wuts' Protective Groups in Organic Synthesis, 3rd Edition, Wiley-InterScience, 1999. Alternatively, other reactions disclosed herein or known in the art will be recognized as having applicability for preparing other compounds of the various embodiments.

Reagents useful for synthesizing compounds may be obtained or prepared according to techniques known in the art.

In the examples described below, unless otherwise indicated, all temperatures in the following description are in degrees Celsius and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated. Tetrahydrofuran (THF) and N,N-dimethylformamide (DMF) were purchased from Aldrich in SureSeal bottles and used as received. All solvents were purified by using standard methods in the art, unless otherwise indicated.

The reactions set forth below were performed under a positive pressure of nitrogen, argon or with a drying tube, at ambient temperature (unless otherwise stated), in anhydrous solvents, and the reaction flasks are fitted with rubber septa for the introduction of substrates and reagents via syringe. Glassware was oven-dried and/or heat-dried. Analytical thin-layer chromatography was performed on glass-backed silica gel 60 F 254 plates (E Merck (0.25 mm)) and eluted with the appropriate solvent ratios (v/v). The reactions were assayed by TLC and terminated as judged by the consumption of starting material.

The TLC plates were visualized by UV absorption or with a p-anisaldehyde spray reagent or a phosphomolybdic acid reagent (Aldrich Chemical, 20 wt % in ethanol) which was activated with heat, or by staining in iodine chamber. Work-ups were typically done by doubling the reaction volume with the reaction solvent or extraction solvent and then washing with the indicated aqueous solutions using 25% by volume of the extraction volume (unless otherwise indicated). Product solutions were dried over anhydrous sodium sulfate prior to filtration, and evaporation of the solvents was under reduced pressure on a rotary evaporator and noted as solvents removed in vacuo. Flash column chromatography [Still et al, J. Org. Chem., 43, 2923 (1978)] was conducted using E Merck-grade flash silica gel (47-61 mm) and a silica gel:crude material ratio of about 20:1 to 50:1, unless otherwise stated. Hydrogenolysis was done at the pressure indicated or at ambient pressure.

Reverse-phase preparative HPLC (RPHPLC) was operated by using a C₁₈ column (5 um, 21.2×150 mm) at flow rate of 20 mL/min and a linear gradient from 5 to 95% of CH₃CN+0.1% TFA (trifluoroacetic acid) over 18 min. High-throughput mass-dependent (reverse-phase HPLC) purification system (HTP) was operated by using a C₁₈ column (5 um, 19×50 mm) at flow rate of 30 mL/min and a linear gradient from 5 to 95% of CH₃CN+0.05% TFA over 9 min. The fractions containing the desire product were lyophilized, or evaporated to dryness under vacuum to provide the dry compound, or evaporated to remove the volatile organic solvent then extracted with organic solvents (ethyl acetate or dichloromethane are commonly used, if necessary, the pH of the aqueous solution could also be adjusted in order to get free base, acid or the neutral compound).

¹H NMR spectra were recorded on a Bruker AV400 instrument operating at 400 MHz, and ¹³C-NMR spectra were recorded operating at 100 MHz. NMR spectra are obtained as CDCl₃ solutions (reported in ppm), using chloroform as the reference standard (7.26 ppm and 77.00 ppm), CD₃OD (3.3 and 49.3 ppm), DMSO-d₆ (2.50 and 39.5 ppm), or an internal tetramethylsilane standard (0.00 ppm) when appropriate. Other NMR solvents were used as needed. When peak multiplicities are reported, the following abbreviations are used: s=singlet, d=doublet, t=triplet, m=multiplet, b or br=broadened, dd=doublet of doublets, dt=doublet of triplets, ft=triplet of triplets. Coupling constants, when given, are reported in Hertz.

Mass spectra were obtained using LC-MS either in ESI or APCI. All melting points are uncorrected.

All final products had greater than 90% purity (by HPLC at wavelengths of 220 nm and 254 nm).

The following examples are intended to illustrate the embodiments disclosed and are not to be construed as being limitations thereto. Additional compounds, other than those described below, may be prepared using the following described reaction scheme or appropriate variations or modifications thereof.

### Synthesis

Scheme 1 illustrates the procedure used for preparing compounds of Formula (I), wherein R¹═R³═H, M=S(O)₂ or C═O.

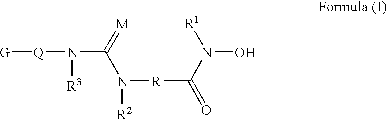

In Scheme 1, R is a linking moiety or equal to —B-A-Z-L- as defined for Formula (2), R″ is R less one CH₂, R¹¹ is a C₁-C₆ alkyl or benzyl, R²¹ is R² less one CH₂ and R² is defined as for Formula (I).

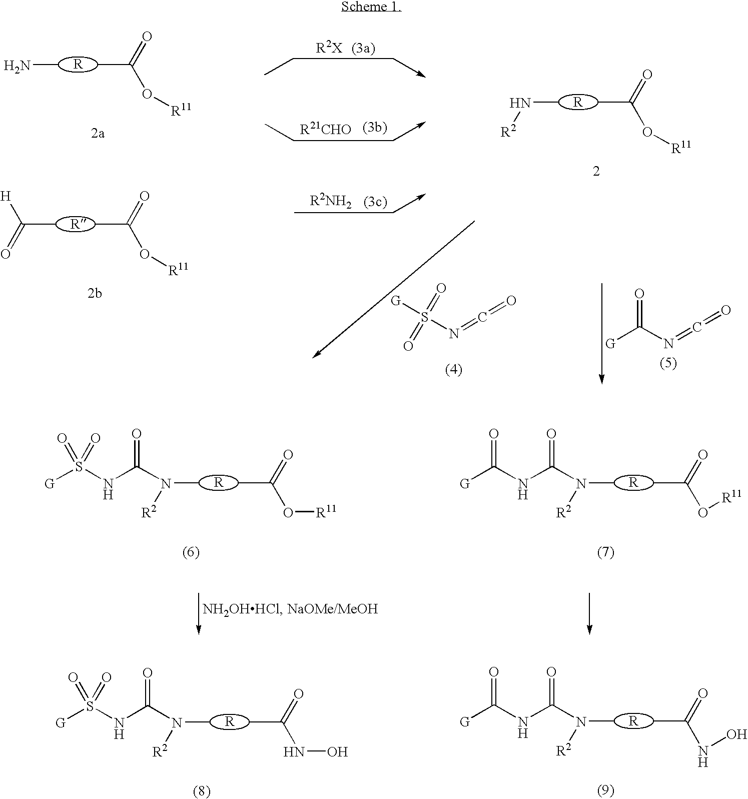

The intermediate (2) in Scheme 1 could be prepared by (i) alkylation of amine (2a) with R²X (3a, X is halo, e.g., I⁻, Br, Cl⁻ or a good leaving group), or (ii) reductive amination of amine (2a) with aldehyde (3b), or (iii) reductive amination of aldehyde (2b) with amine R²NH₂ (3c).

### Synthesis of Sulfonylurea Linked Hydroxamic Acid (8).

Scheme 1. Amine (2) or Amine (2a, for R²═H) is reacted with sulfonylisocyanate (4) to give sulfonylurea (6) which is subsequently converted to hydroxamic acid (8) by amination of the ester with hydroxylamine.

Sulfonylurea linked hydroxamic acid (8) could also be synthesized by a synthetic route as described in Scheme 2. R¹² is a protecting group; examples are benzyl, 2,4-dimethoxy-benzyl, tetrahydro-pyran-2-yl and tert-Butyl-dimethyl-silyl.

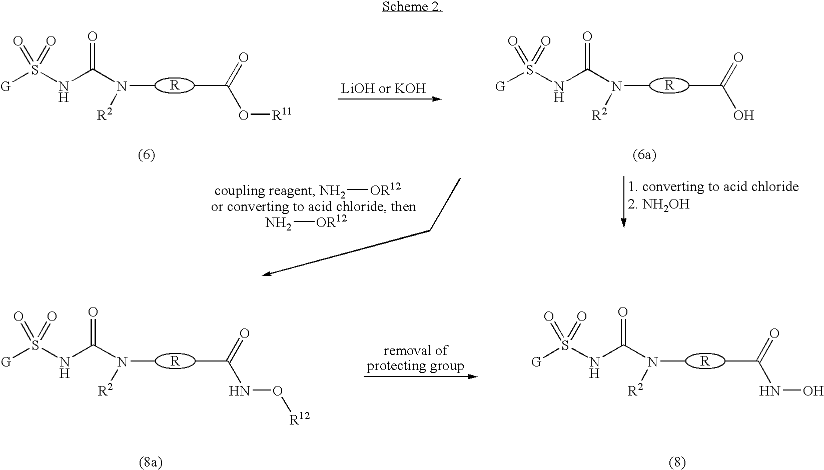

The ester (6) is hydrolyzed to the acid (6a). The acid is subsequently converted to the hydroxamic acid (8) by either Method A or Method B.

Method A. (i) the acid is converted to acid chloride by treating it with ClCOCOCl, or SOCl₂, or other reagents under neutral conditions (such as Ph₃P with CBr₄, or 2,4,6-Trichloro-[1,3,5]triazine); or (ii) the acid is converted to an active ester by reacting it with isobutyl chloroformate; (iii) the acid chloride or active ester is reacted with hydroxylamine or the O-protected hydroxylamine [e.g., O-benzylhydroxylamine, O-(2,4-dimethoxy-benzyl)-hydroxylamine, O,N-bis-(2,4-dimethoxy-benzyl)-hydroxylamine, O-(tetrahydro-pyran-2-yl)-hydroxylamine, O-(tert-butyl-dimethyl-silyl)-hydroxylamine] to give the hydroxamic acid or the C-protected hydroxamic acid in which the protecting group is subsequently removed by methods known in the literature such as hydrogenolysis to remove the benzyl group or acidic cleavage to cleave the acid labile protecting groups.

Method B. Coupling the acid with hydroxylamine or C-protected hydroxylamine (R¹²ONH₂) with a coupling reagent, then followed by removing the protecting group by methods known in the literature.

### Synthesis of Acylurea Linked Hydroxamic Acid (9).

Acylurea linked hydroxamic acid (9) could be synthesized by methods analogous to those used for synthesis of sulfonylurea linked hydroxamic acid (8).

Scheme 1. Amine (2) or Amine (2a, for R²═H) is reacted with acylisocyanate (5) to give acylurea (7) which is subsequently converted to hydroxamic acid (9) by amination of the ester with hydroxylamine.

Acylurea linked hydroxamic acid (9) could also be synthesized by method described in Scheme 3.

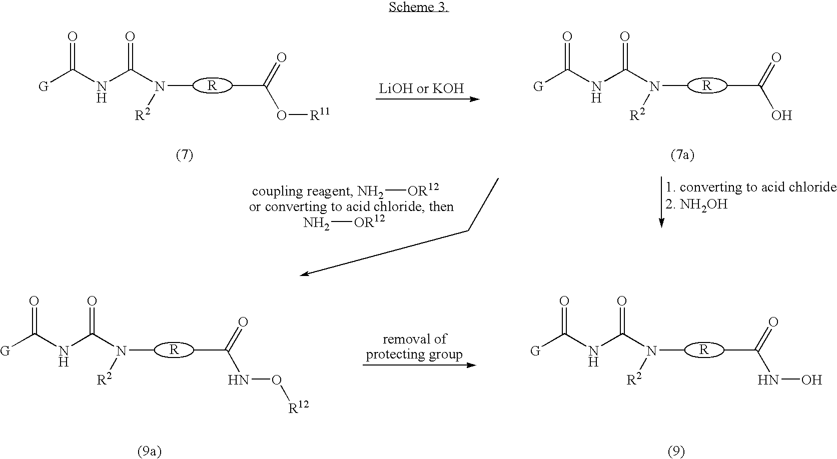

Furthermore, sulfonylurea linked hydroxamic acid (8) and acylurea linked hydroxamic acid (9) could also be synthesized by a synthetic route described in Scheme 4. O-protected hydroxamate starting material amine (2c) or aldehyde (2d) are used to make the O-protected hydroxamate intermediate (2P) which is subsequently converted to the corresponding sulfonylurea (8a) and acylurea (9a). After removal the protecting group, sulfonylurea (8) and acylurea (9) are obtained.

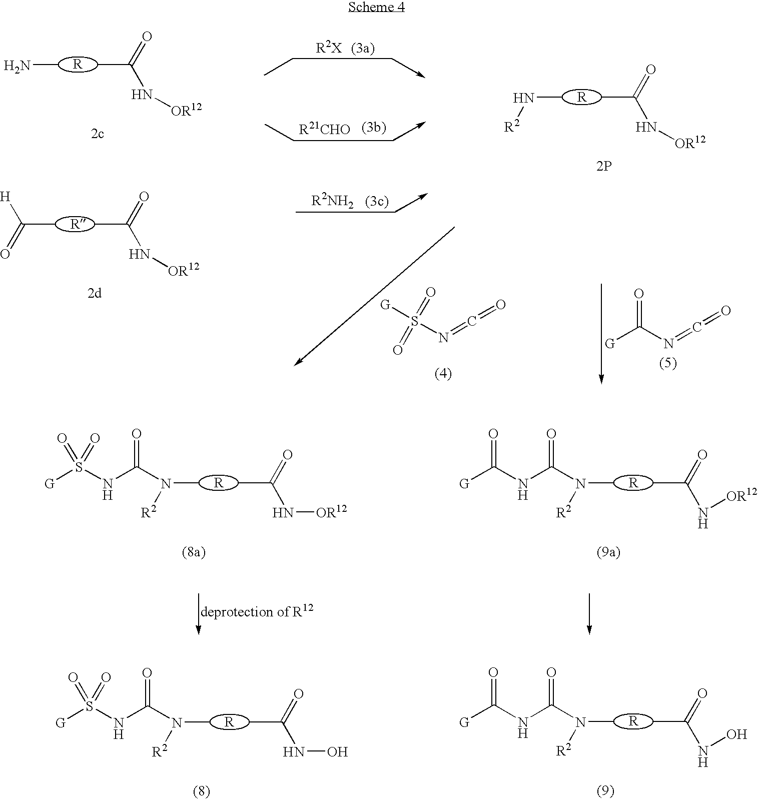

Scheme 5 illustrates the procedure used for preparing compounds of Formula (I), wherein R¹═H, M=S(O)₂. R¹³ is selected from R¹¹ or R¹².

Due to acidity of the sulfonylurea (6), the R³ group could be introduced by alkylation of 6 with R³X (X═I⁻, Br or Cl⁻) or by reacting with R³OH under Mitsunobu reaction condition. The product (6b) could be converted to the hydroxamic acid (8b) by using the similar condition as described for (8) in Scheme 1, 2 or 4.

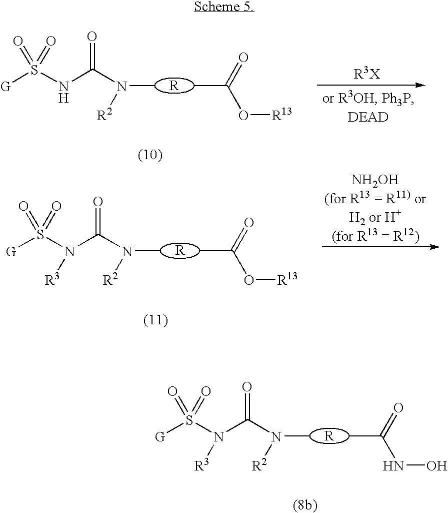

The following preparation and examples are given to enable those skilled in the art to more clearly understand and to practice the subject matter hereof. They should not be considered as limiting the scope of the invention, but merely as being illustrative and representative thereof.

### Intermediate 1

### Preparation of 6-[3-(Benzenesulfonyl)ureido]-hexanoic acid methyl ester

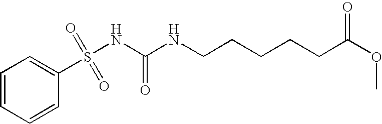

To a mixture of 6-Amino-hexanoic acid methyl ester hydrochloride (0.10 g, 0.5 mmol), triethylamine (0.12 g, 1.2 mmol, 0.17 mL) and DMAP (0.06 g, 0.05 mmol) in the presence of CH₂Cl₂ (5 mL) was added phenyl sulfonyl isocyanate (0.12 g, 0.6 mmol). The reaction mixture was stirred at room temperature for 4 days. The reaction mixture was diluted with water (10 mL) and extracted with CH₂Cl₂ (3×10 mL). The combined organic extracts were dried over MgSO₄, filtered and the solvent was removed in vacuo. The crude residue was chromatographed (silica) with 1-10% MeOH in CH₂Cl₂ to give 6-[3-(Benzenesulfonyl)ureido]-hexanoic acid methyl ester (0.1 g, 0.3 mmol, 58%) as a colorless oil which solidified on standing.

¹H NMR (CDCl₃) δ7.95-7.89 (2H, m), 7.66-7.53 (3H, m), 3.67 (3H, s), 3.22 (2H, q, J=6.9 Hz), 2.29 (2H, t, J=7.4 Hz), 1.62 (2H, m, J=7.4 Hz), 1.50 (2H, m, J=7.4 Hz) and 1.31-1.27 (2H, m).

### Intermediate 2

### Preparation of 4-[3-(toluene-4-sulfonyl)ureidomethyl-benzoic acid methyl ester

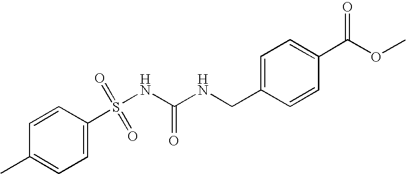

Proceeding as described in Intermediate 1 above but using appropriate starting materials. Yield: 67%. Light yellow solid; LC-MS (ESI, positive mode) m/z 363 ([M+H]⁺).

¹H NMR (DMSO-d₆) δ 10.78 (bs, 1H), 7.85 (d, 2H, J=8.2 Hz), 7.78 (d, 2H, J=8.2 Hz), 7.40 (d, 2H, J=8.0 Hz), 7.23 (d, 2H, J=8.1 Hz), 4.22 (d, 2H, J=5.9 Hz), 3.84 (s, 3H), 2.40 (s, 3H).

### Intermediate 3

### Preparation of 6-(3-Benzoyl-ureido)-hexanoic acid methyl ester

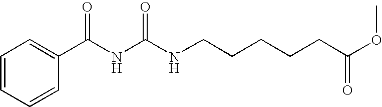

To a mixture of 6-Amino-hexanoic acid methyl ester hydrochloride (0.05 g, 0.27 mmol), triethylamine (0.069 g, 0.6 mmol, 0.096 mL) and DMAP (0.03 g, 0.027 mmol) in the presence of CH₂Cl₂ (2 mL) was added benzoyl isocyanate (0.048 g, 0.3 mmol). The reaction mixture was stirred at room temperature for 4 days. The reaction mixture was diluted with water (10 mL) and extracted with CH₂Cl₂ (3×10 mL). The combined organic extracts were dried over MgSO₄, filtered and the solvent was removed in vacuo. The crude residue was chromatographed with 1-10% MeOH in CH₂Cl₂ to give 6-(3-Benzoyl-ureido)-hexanoic acid methyl ester (0.087 g, 0.2 mmol, quantitative yield) as a colorless oil which solidified on standing.

¹H NMR (CDCl₃) δ 8.66 (br s, 2H), 7.89-7.87 (m, 2H), 7.62-7.48 (m, 3H), 3.67 (s, 3H), 3.39 (q, 2H, J=7.0 Hz), 2.33 (t, 2H, J=7.4 Hz), 1.70-1.60 (m, 4H) and 1.45-1.40 (m, 2H).

### Large Scale

To a mixture of 6-Amino-hexanoic acid methyl ester hydrochloride (0.363 g, 2.00 mmol), triethylamine (0.558 mL, 4.00 mmol) and DMAP (0.024 g, 0.20 mmol) in the presence of CH₂Cl₂ (10 mL) was added benzoyl isocyanate (0.276 mL, 2.20 mmol). The reaction mixture was stirred at room temperature for 6 h. The reaction mixture was added brine and extracted with 10% methanol in dichloromethane. The extract was dried and concentrated and purified by reverse-phase preparative HPLC to give 6-(3-Benzoyl-ureido)-hexanoic acid methyl ester (0.459 g, 79%).

### Intermediate 4

### Preparation of 6-(3-Benzoyl-ureido)-hexanoic acid

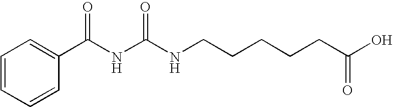

To a solution of 6-(3-Benzoyl-ureido)-hexanoic acid methyl ester (0.043 g, 0.14 mmol) in dry MeOH (2 mL) was added NH₂OH.HCl (0.015 g, 0.2 mmol) followed by NaOMe (0.08 mL, 5.38M, 0.4 mmol). The reaction mixture was stirred at room temperature under nitrogen for 2 hours, and then was diluted with acetonitrile and the solvent was removed in vacuo. The crude residue was purified by mass induced HTP. No hydroxamic acid was obtained but the corresponding 6-(3-Benzoyl-ureido)-hexanoic acid was obtained as a white fluffy solid.

¹H NMR (DMSO-d₆) δ 10.6 (1H, s), 8.60 (1H, bs), 7.92-7.90 (2H, m), 7.58-7.55 (1H, m), 7.47-7.43 (2H, m), 3.20-3.15 (2H, m), 2.16 (2H, t, J=7.3 Hz), 1.52-1.42 (4H, m) and 1.30-1.24 (2H, m).

### Intermediate 5

### Preparation of 4-(3-Benzoyl-ureidomethyl)-benzoic acid methyl ester

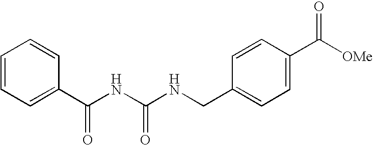

To a solution of 4-Aminomethyl-benzoic acid methyl ester hydrochloride (0.425 g, 2.11 mmol), triethylamine (0.60 mL, 0.4.31 mmol) and DMAP (0.020 g, 0.16 mmol) in CH₂Cl₂ (10 mL) was added benzoyl isocyanate (Sigma-Aldrich, 90% pure, 0.413 g, 0.2.53 mmol). The reaction mixture was stirred at room temperature for 2.5 h and was evaporated to dryness. The white residue was added water, filtered and the solid was washed with water (x4) and dried. 4-(3-Benzoyl-ureidomethyl)-benzoic acid methyl ester was obtained as white solid (0.586 g, 89%). HPLC purity at 254 nm: 99.7%; LC-MS (ESI, positive mode) m/z 313 ([M+H]⁺);

¹H NMR (DMSO-d₆) δ 9.12 (1H, t, J=5.4 Hz), 8.68 (1H, s), 8.04 (2H, dt, J=8.4, 1.8 Hz), 7.89 (2H, dt, J=8.2, 1.6 Hz), 7.62 (1H, tt, J=7.4, 1.8 Hz), 7.50 (2H, t, J=8.0 Hz), 7.44 (2H, d, J=8.4 Hz), 4.64 (2H, d, J=6.0 Hz), 3.93 (3H, s).

### Intermediate 6

### Preparation of 3-(4-{3-Benzoyl-1-[2-(1H-indol-3-yl)-ethyl]-ureidomethyl}-phenyl)-acrylic acid methyl ester

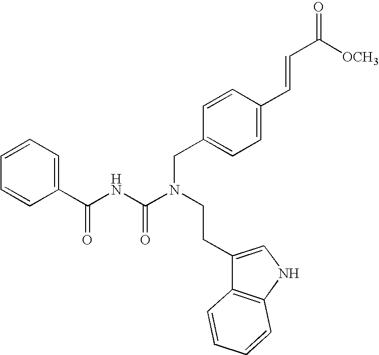

To a solution of 3-(4-{[2-(1H-Indol-3-yl)-ethylamino]-methyl}-phenyl)-acrylic acid methyl ester (0.100 g, 0.300 mmol), triethylamine (0.063 mL, 0.45 mmol) and DMAP (0.004 g, 0.03 mmol) in CH₂Cl₂ (3 mL) was added benzoyl isocyanate (90% pure, 0.045 mL, 0.36 mmol). The reaction mixture was stirred at room temperature for 22 h and was extracted with ethyl acetate. The extract was dried (MgSO₄) and concentrated. The residue was purified by HTP. 3-(4-{3-Benzoyl-1-[2-(1H-indol-3-yl)-ethyl]-ureidomethyl}-phenyl)-acrylic acid methyl ester was obtained as pale yellow solid (0.072 g, 50%).

LC-MS (ESI, positive mode) m/z 482 ([M+H]⁺);

¹H NMR (DMSO-d₆) δ 10.82 (1H, s), 10.25 (1H, bs), 7.83 (2H, d, J=8.1 Hz, PhH), 7.74 (1H, d, J=16.0 Hz), 7.61 (1H, t, J=7.3 Hz), 7.50 (2H, t, J=7.5 Hz), 7.45 (2H, br d like), 7.38-7.35 (1H, br m), 7.31 (1H, d, J=8.1 Hz), 7.10 (1H, bs like), 7.02 (1H, t, J=7.2 Hz), 6.90-6.70 (1H, very broad s), 6.66 (1H, d, J=16.0 Hz), 4.69 (2H, s), 3.73 (3H, s), 3.52 (2H, t, J=7.7 Hz), 2.97 (2H, t, J=7.5 Hz); ¹³C NMR (DMSO-d₆) δ 166.7, 166.4, 154.1 (br), 144.2, 140.3, 136.1, 133.2, 133.0, 132.2, 128.5, 128.4, 127.9, 127.8, 126.9, 122.9, 120.9, 118.2, 118.0, 117.6, 111.4, 110.7, 51.4, 49.5*, 49.3*, 24.4* (*: very broad and weak peaks, identified by ¹H-¹³C HSQC).

### Intermediate 7

### Preparation of 8-Amino-octanoic acid methyl ester hydrochloride

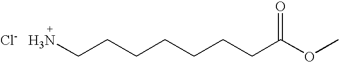

To a 100 mL round-bottomed flask, 8-Amino-octanoic acid (2.116 g, 13.29 mmol) and methanol (50 mL) were added. The mixture was stirred and cooled in a dry-ice/acetone bath under nitrogen. SOCl₂ (1.5 mL, 20.7 mmol) was added via syringe, then the dry-ice bath was removed and the mixture was stirred at room temperature for 2.5 h. The solution was evaporated and the residue was added diethyl ether. The solid was filtered and dried under vacuum. 8-Amino-octanoic acid methyl ester hydrochloride was obtained as white solid (2.772 g, 99.8%). LC-MS (ESI, positive mode) m/z 376 ([M−Cl]⁺). ¹H NMR (DMSO-d₆) δ 8.24 (3H, s, NH₃⁺), 3.67 (3H, s, OCH₃), 3.00 (2H, m), 2.30 (2H, t, J=7.5 Hz), 1.78 (2H, penta, J=7.3 Hz), 1.61 (2H, penta, J=7.2 Hz), 1.41 (2H, m), 1.39-1.32 (4H, m); ¹³C NMR (DMSO-d₆) δ174.1, 51.4, 39.9, 33.9, 28.6, 27.5, 26.3, 24.7.

### Intermediate 8

### Preparation of 8-[3-(4-methylbenzenesulfonyl)ureido]octanoic acid methyl ester

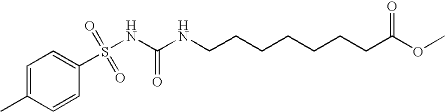

To a mixture of 8-Amino-octanoic acid methyl ester hydrochloride (0.601 g, 2.865 mmol), triethylamine (1.0 mL, 7.18 mmol) and DMAP (0.0313 g, 0.256 mmol) in CH₂Cl₂ (20 mL) was added p-toluene sulfonylisocyanate (0.63 mL, 4.12 mmol). The reaction mixture was stirred at room temperature for 19.5 h. The reaction mixture was diluted with 1N HCl and extracted with CH₂Cl₂ (100 mL×1, 50 mL×2). The combined organic extracts were dried over MgSO₄, filtered and the solvent was removed in vacuo. The crude residue was chromatographed (silica) with 2-10% MeOH in CH₂Cl₂ to give 8-[3-(4-methylbenzenesulfonyl)ureido]octanoic acid methyl ester (0.730 g, 69%) as a white solid.

LC-MS (ESI, positive mode) m/z 371 ([M+H]⁺).

¹H NMR (CDCl₃) δ 8.80 (1H, bs), 7.78 (2H, d, J=8.3 Hz), 7.31 (2H, d, J=8.1 Hz), 6.52 (1H, t, J=5.2 Hz), 3.67 (3H, s, OCH₃), 3.19 (2H, q, J=6.6 Hz), 2.44 (3H, s, Ar—CH₃), 2.30 (2H, t, J=7.5 Hz), 1.63-1.58 (2H, m), 1.48-1.42 (2H, m), 1.31-1.22 (6H, m); ¹³C NMR (DMSO-d₆) δ174.3, 151.9, 144.6, 136.8, 129.6, 127.0, 51.5, 40.2, 34.0, 29.4, 28.9, 28.8, 26.6, 24.8, 21.6

### Intermediate 9

### Preparation of 8-[3-methyl-3-(4-methylbenzenesulfonyl)ureido]octanoic acid methyl ester

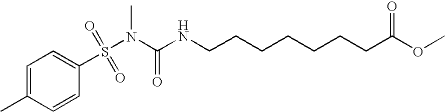

To a mixture of 8-[3-(4-methylbenzenesulfonyl)ureido]octanoic acid methyl ester (0.161 g, 0.435 mmol), K₂CO₃ (0.572 g, 4.14 mmol) and acetonitrile (4 mL) was added MeI (0.270 mL, 4.35 mmol). The reaction mixture was stirred at room temperature under nitrogen for 17 h. The reaction mixture was diluted with 1N HCl and extracted with ethyl acetate (Na₂S₂O₃ was added to the aqueous layer to reduce the 12). The combined organic extracts were dried over MgSO₄, filtered and the solvent was removed in vacuo. ¹H NMR of the crude residue (0.166 g) showed that the molar ratio of 8-[3-4-methylbenzenesulfonyl)ureido]octanoic acid methyl ester to degraded product 4,N,N-Trimethyl-benzenesulfonamide was 3:1.

LC-MS (ESI, positive mode) m/z 385 ([M+H]⁺).

¹H NMR (CDCl₃) δ 7.70 (2H, d, J=8.3 Hz), 7.34 (2H, d, J=8.5 Hz), 3.66 (3H, s, OCH₃), 3.24 (2H, q, J=5.7 Hz), 3.12 (3H, s, NCH₃), 2.43 (3H, s, Ar—CH₃), 2.31 (2H, t, J=7.5 Hz), 1.65-1.60 (2H, m), 1.55-1.51 (2H, m), 1.33-1.26 (6H, m).

### Intermediate 10

### Preparation of 8-(3-Benzoyl-ureido)-octanoic acid methyl ester

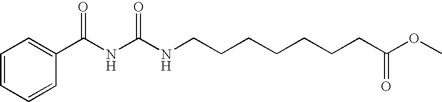

To a solution of 8-Amino-octanoic acid methyl ester hydrochloride (0.423 g, 2.02 mmol), triethylamine (0.56 mL, 4.02 mmol) and DMAP (0.022 g, 0.18 mmol) in CH₂Cl₂ (10 mL) was added benzoyl isocyanate (90% pure, 0.370 g, 2.26 mmol). The reaction mixture was stirred at room temperature for 2 h and was added silica gel and filtered through silica and washed with ethyl acetate. The filtrate was evaporated to dryness to give colorless oil (0.691 g, 106%), which was solidified at room temperature under vacuum.

LC-MS (ESI, positive mode) m/z 321 ([M+H]⁺).

¹H NMR (CDCl₃) δ 10.52 (1H, s), 8.92 (1H, t, J=5.6 Hz), 8.09 (2H, d, J=7.2 Hz), 7.56 (1H, t like), 7.45 (2H, t, J=7.7 Hz), 3.64 (3H, s, OCH₃), 3.35 (2H, q, J=6.0 Hz), 2.29 (2H, t, J=7.5 Hz), 1.63-1.56 (4H, m), 1.39-1.32 (6H, m); ¹³C NMR (CDCl₃) δ 173.6, 168.1, 154.6, 132.3, 132.0, 128.0, 127.7, 50.9, 39.3, 33.5, 29.0, 28.5, 28.4, 26.3, 24.3.

### Intermediate 11

### Preparation of 7-Amino-heptanoic acid methyl ester hydrochloride

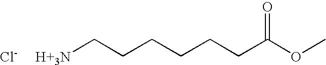

Proceeding as described in Intermediate 7 above but using appropriate starting materials (7-Amino-heptanoic acid), the titled compound was prepared as white solid (0.490 g, 100%).

### Intermediate 12

### Preparation of 7-(3-Benzoyl-ureido)-heptanoic acid methyl ester

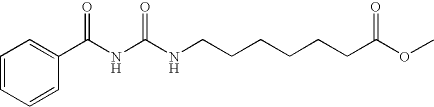

Proceeding as described in Intermediate 10 above but using appropriate starting materials (7-Amino-heptanoic acid methyl ester hydrochloride), the crude titled compound was obtained as oil which was solidified under vacuum and could be used for next step of reaction without further purification. LC-MS (ESI, positive mode) m/z 307 ([M+H]⁺).

¹H NMR (CDCl₃) δ 10.30 (1H, s), 8.90 (1H, t, J=5.4 Hz), 8.05 (2H, d, J=7.5 Hz), 7.57 (1H, t, J=7.4 Hz), 7.47 (2H, d, J=7.6 Hz), 3.65 (3H, s, OCH₃), 3.35 (2H, q, J=6.6 Hz), 2.30 (2H, t, J=7.5 Hz), 1.68-1.56 (4H, m), 1.45-1.35 (4H, m); ¹³C NMR (CDCl₃) δ 173.6, 168.1, 154.5, 132.4, 132.0, 128.1, 127.6, 51.0, 39.3, 33.5, 28.9, 28.3, 26.1, 24.3.

### Intermediate 13

### Preparation of a salt of 6-(3-Phenyl-propylamino)-hexanoic acid methyl ester

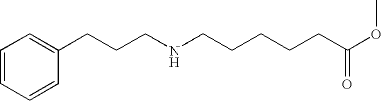

To a 100 mL flask, 6-Amino-hexanoic acid methyl ester hydrochloride (0.555 g, 3.06 mmol), NaBH(OAc)₃ (0.782 g, 3, 69 mmol), 3-Phenyl-propionaldehyde (0.47 mL, 3.21 mmol), dichloromethane (10 mL) and triethylamine (0.43 mL, 3.09 mmol) were added. The above mixture was sonicated for 1 min then stirred at room temperature overnight. The reaction mixture was added aqueous Na₂CO₃ and extracted with dichloromethane (x 2). The extract was dried and purified by reverse-phase preparative HPLC to give the titled compound as an oil (0.202 g, 30% calculated as TFA salt).

LC-MS (ESI, positive mode) m/z 264 ([M+H]⁺).

¹H NMR (CDCl₃) δ 11.82 (1H, s), 8.62 (2H, s, —NH₂⁺—), 7.26 (2H, t, J=7.3 Hz), 7.18 (1H, t, J=7.3 Hz), 7.11 (2H, d, J=7.0 Hz), 3.64 (3H, s, OCH₃), 2.94 and 2.92 (each of 2H, overlapped, identified by COSY), 2.63 (2H, t, J=7.5 Hz), 2.26 (2H, t, J=7.3 Hz), 2.00 (2H, penta, J=7.6 Hz), 1.65 (2H, penta, J=7.5 Hz), 1.55 (2H, penta, J=7.7 Hz), 1.33 (2H, m); ¹³C NMR (CDCl₃) δ 173.6, 139.2, 128.2, 127.7, 126.0, 51.1, 47.3, 47.1, 32.9, 32.0, 26.9, 25.2, 25.0, 23.4.

### Intermediate 14

### Preparation of 6-[3-Benzoyl-1-(3-phenyl-propyl)-ureido]-hexanoic acid methyl ester

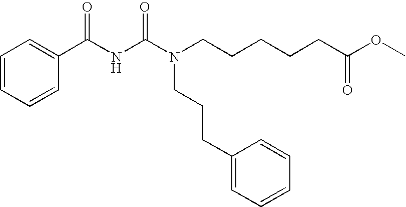

Proceeding as described in Intermediate 10 above but using appropriate starting materials (a salt of 6-(3-Phenyl-propylamino)-hexanoic acid methyl ester with 2 TFA), the crude titled compound was purified by reverse-phase preparative HPLC and flash chromatography (silica, 5% Methanol in dichloromethane) to give the pure compound as gum (0.063 g, 43%). LC-MS (ESI, positive mode) m/z 411 ([M+H]⁺).

¹H NMR (CDCl₃) δ 8.20 (1H, bs), 7.78 (2H, bs), 7.54 (1H, t, J=7.4 Hz), 7.43 (2H, t, J=7.6 Hz), 7.24 (2H, d, J=7.2 Hz), 7.18-7.13 (3H, m), 3.65 (3H, s), 3.36 [4H, m or 3.38 (2H, m) and 3.36 (2H, m)], 2.64 (2H, t, J=7.4 Hz), 2.30 (2H, t, J=7.4 Hz), 1.96 (2H, penta, J=7.4 Hz), 1.66-1.56 (4H, m), 1.31 (2H, m); ¹³C NMR (CDCl₃) 173.5, 165.8 (br), 153.4 (br), 140.7 (br), 132.7, 132.1, 128.1, 128.0, 127.9, 127.3, 125.6, 51.0, 46.9 (br, 2×CH₂N), 33.4, 32.4, 28.9, 27.1 (br), 25.8, 24.1.

### Intermediate 15

### Preparation of 4-{[2-(1H-Indol-3-yl)-ethylamino]-methyl}benzoic acid methyl ester

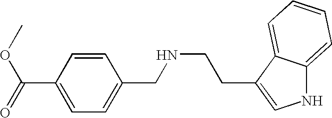

To a 250 mL flask, tryptamine hydrochloride (0.582 g, 2.96 mmol), 4-Formyl-benzoic acid methyl ester (0.488 g, 2.97 mmol), dichloromethane (25 mL), methanol (10 mL) and triethylamine (0.50 mL, 3.59 mmol) were added and the mixture was stirred at room temperature for 4 h, then evaporated to dryness. The residue was dissolved in dichloromethane (25 mL), added NaBH(OAc)₃ (0.805 g, 3.80 mmol) and stirred at room temperature overnight. The mixture was added aqueous NaHCO₃, extracted with dichloromethane (×3) and dried (MgSO₄). The residue was purified by reverse-phase preparative HPLC and the desire fractions were combined and evaporated to remove the organic solvent. The resultant solution was neutralized with aqueous NaHCO₃ and extracted with dichloromethane (×3), dried (MgSO₄) to give the titled compound as a gum (0.437 g, 48%). LC-MS (ESI, positive mode) m/z 309 ([M+H]⁺).

¹H NMR (CDCl₃) δ 8.24 (1H, s), 7.95 (2H, d, J=8.3 Hz), 7.59 (1H, d, J=7.9 Hz), 7.32 (3H, overlapped by CHx2 and CH, d, J=8.2 Hz), 7.18 (1H, td, J=7.5, 1.1 Hz), 7.10 (1H, td, J=7.5, 1.0 Hz), 6.98 (1H, d, J=2.3 Hz), 3.89 (3H, s), 3.84 (2H, s), 2.99-2.95 (4H, m); ¹³C NMR (CDCl₃) δ 166.7, 145.4, 136.0, 129.2, 128.3, 127.5, 127.0, 121.6 (two CH overlapped), 118.8, 118.4, 113.3, 110.8, 53.0 (CH₂N), 51.6 (OCH₃), 48.9, 25.3.

### Intermediate 16

### Preparation of 4-{[(Pyridin-3-ylmethyl)-amino]-methyl}-benzoic acid methyl ester

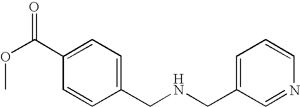

Proceeding as described in INTERMEDIATE 15 above but using appropriate starting materials 3-(aminomethyl)pyridine and 1 equivalent of acetic acid (neither triethylamine nor methanol was added). After workup, the crude extract was used for further step of reaction without further purification.

LC-MS (ESI, positive mode) m/z 257 ([M+H]⁺).

### Intermediate 17

### Preparation of 4-(Benzylamino-methyl)-benzoic acid methyl ester

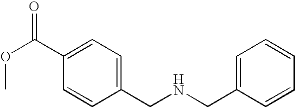

Proceeding as described in INTERMEDIATE 16 above but using appropriate starting material benzylamine. After workup, the crude extract was used for further step of reaction without further purification.

LC-MS (ESI, positive mode) m/z 256 ([M+H]⁺).

### Intermediate 18

### Preparation of 4-[(3-Hydroxy-propylamino)-methyl]-benzoic acid methyl ester

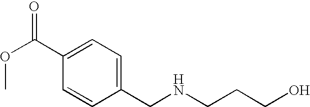

Proceeding as described in INTERMEDIATE 16 above but using appropriate starting material 3-Amino-propan-1-ol. After workup, the crude extract was used for further step of reaction without further purification.

LC-MS (ESI, positive mode) m/z 224 ([M+H]⁺).

### Intermediate 19

### Preparation of 4-[(2-Pyridin-2-yl-ethylamino)-methyl]-benzoic acid methyl ester

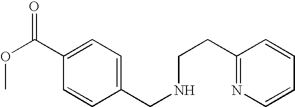

Proceeding as described in INTERMEDIATE 16 above but using appropriate starting material 2-Pyridin-2-yl-ethylamine. After workup, the crude extract was used for further step of reaction without further purification.

LC-MS (ESI, positive mode) m/z 271 ([M+H]⁺).

### Intermediate 20

### Preparation of 4-(Phenethylamino-methyl)-benzoic acid methyl ester

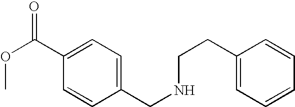

Proceeding as described in INTERMEDIATE 16 above but using appropriate starting materials phenethylamine. After workup, the crude extract was used for further step of reaction without further purification.

LC-MS (ESI, positive mode) m/z 270 ([M+H]⁺).

### Intermediate 21

### Preparation of 3-(4-{[(Pyridin-3-ylmethyl)-amino]-methyl}phenyl)-acrylic acid methyl ester

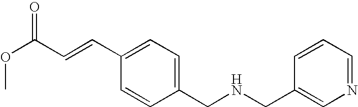

Proceeding as described in INTERMEDIATE 16 above but using appropriate starting materials 3-(4-Formyl-phenyl)-acrylic acid methyl ester, 3-(aminomethyl)pyridine. After workup, the crude extract was used for further step of reaction without further purification. LC-MS (ESI, positive mode) m/z 283 ([M+H]⁺).

### Intermediate 22

### Preparation of 3-{4-[(2-Pyridin-2-yl-ethylamino)-methyl]-phenyl}acrylic acid methyl ester

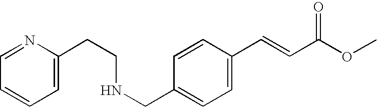

Proceeding as described in INTERMEDIATE 16 above but using appropriate starting materials 3-(4-Formyl-phenyl)-acrylic acid methyl ester and 2-Pyridin-2-yl-ethylamine. After workup, the crude extract was used for further step of reaction without further purification. LC-MS (ESI, positive mode) m/z 297 ([M+H]⁺).

### Intermediate 23

### Preparation of 3-{4-[(3-Hydroxy-propylamino)-methyl]-phenyl}-acrylic acid methyl ester

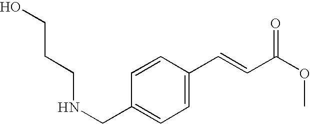

Proceeding as described in INTERMEDIATE 16 above but using appropriate starting materials. 3-(4-Formyl-phenyl)-acrylic acid methyl ester and 3-Amino-propan-1-ol. After workup, the crude extract was used for further step of reaction without further purification. LC-MS (ESI, positive mode) m/z 250 ([M+H]⁺).

### Intermediate 24

### Preparation of 3-{4-[3-Benzoyl-1-(3-hydroxy-propyl)-ureidomethyl]-phenyl}-acrylic acid methyl ester

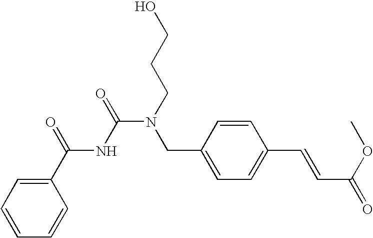

Proceeding as described in INTERMEDIATE 6 above but using appropriate starting materials. LC-MS (ESI, positive mode) m/z 397 ([M+H]⁺).

### Intermediate 25

### Preparation of 3-{4-[3-Benzoyl-1-(2-pyridin-2-yl-ethyl)-ureidomethyl]-phenyl}-acrylic acid methyl ester

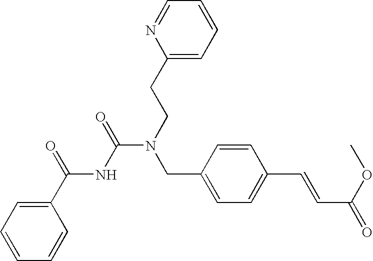

Proceeding as described in INTERMEDIATE 6 above but using appropriate starting materials. LC-MS (ESI, positive mode) m/z 444 ([M+H]⁺).

### Intermediate 26

### Preparation of 3-[4-(3-Benzoyl-1-pyridin-3-ylmethyl-ureidomethyl)-phenyl]-acrylic acid methyl ester

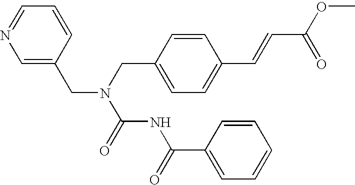

Proceeding as described in INTERMEDIATE 6 above but using appropriate starting materials. Yield: 67%. LC-MS (ESI, positive mode) m/z 430 ([M+H]⁺).

¹H NMR (CDCl₃) δ 9.68 (1H, bs), 8.49 (1H, d, J=3.3 Hz), 8.44 (1H, s), 7.86 (2H, d, J=7.5 Hz), 7.69 (1H, m), 7.66 (1H, d, J=16.0 Hz), 7.50 (1H, J=7.3 Hz), 7.47 (2H, d, J=8.2 Hz), 7.38 (2H, t, J=7.7 Hz), 7.27-7.23 (3H, m), 6.42 (1H, d, J=16.0 Hz), 4.57 (2H, s), 4.56 (2H, s), 3.79 (3H, s); ¹³C NMR (CDCl₃) δ 166.8, 166.6, 155.3, 148.7, 148.5, 143.6, 137.8, 133.5, 132.3, 132.2, 123.3, 117.6, 51.2, 50.5, 47.6.

### Intermediate 27

### Preparation of 4-{3-Benzoyl-1-[2-(1H-indol-3-yl)-ethyl]-ureidomethyl}benzoic acid methyl ester

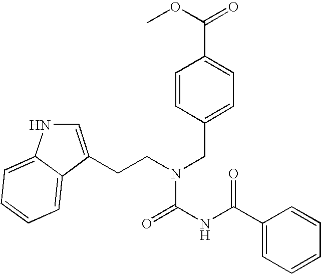

Proceeding as described in INTERMEDIATE 6 above but using appropriate starting materials. Yield 65%. LC-MS (ESI, positive mode) m/z 456 ([M+H]⁺).

¹H NMR (CDCl₃) δ 9.08 (1H, bs), 7.96 (2H, d, J=8.2 Hz), 7.45-7.05 [10H, including 7.43 (2H, d, J=7.6 Hz), 7.17 (2H, bs), 7.11 (1H, t, J=7.6 Hz)], 6.98 (1H, t, J=7.4 Hz), 6.91 (1H, bs), 4.65 (2H, s), 3.87 (3H, s), 3.57 (2H, br t like, J=5.8 Hz), 2.98 (2H, br t, J=5.4 Hz); ¹³C NMR (CDCl₃) δ 169.3, 166.0, 153.9, 141.7, 136.1, 132.3; 131.9, 129.6, 129.0, 128.0, 127.3, 127.0, 126.2, 123.0, 121.7, 1191.1, 117.6, 111.4, 110.7, 51.7, 49.3 (identified by HSQC), 48.2, 23.2.

### Intermediate 28

### Preparation of 4-[3-Benzoyl-1-(3-hydroxy-propyl)-ureidomethyl]-benzoic acid methyl ester

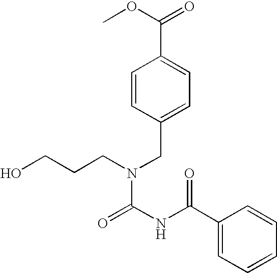

Proceeding as described in INTERMEDIATE 6 above but using appropriate starting materials. LC-MS (ESI, positive mode) m/z 371 ([M+H]⁺).

### Intermediate 29

### Preparation of 4-[3-Benzoyl-1-(2-pyridin-2-yl-ethyl)-ureidomethyl]-benzoic acid methyl ester

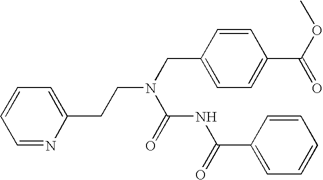

Proceeding as described in INTERMEDIATE 6 above but using appropriate starting materials. LC-MS (ESI, positive mode) m/z 418 ([M+H]⁺).

### Intermediate 30

### Preparation of 4-(3-Benzoyl-1-pyridin-3-ylmethyl-ureidomethyl)-benzoic acid methyl ester

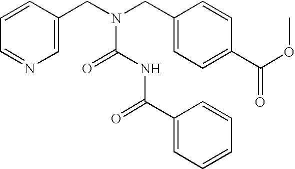

Proceeding as described in INTERMEDIATE 6 above but using appropriate starting materials. LC-MS (ESI, positive mode) m/z 404 ([M+H]⁺).

### Intermediate 31

### Preparation of 4-(3-Benzoyl-ureido)-butyric acid ethyl ester

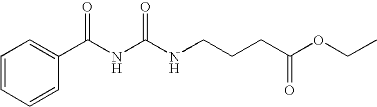

Proceeding as described in INTERMEDIATE 6 above but using appropriate starting materials. LC-MS (ESI, positive mode) m/z 279 ([M+H]⁺).

¹H NMR (CDCl₃) δ 10.38 (1H, s), 8.96 (1H, t, J=5.6 Hz, 8.06 (2H, t, J=7.5 Hz), 7.58 (1H, t, J=7.4 Hz), 7.48 (2H, t, J=7.6 Hz), 4.12 (2H, q, J=7.1 Hz), 3.42 (2H, td, J=6.7 and 6.3 Hz), 2.40 (2H, t, J=7.4 Hz), 1.95 (2H, penta, J=7.2 Hz); ¹³C NMR (CDCl₃) δ 172.5, 168.1, 154.7, 132.5, 131.9, 128.1, 127.6, 59.9, 38.7, 31.1, 24.5, 13.7.

### Intermediate 32

### Preparation of 4-piperazin-1-ylmethyl-benzoic acid methyl ester (2*TFA salt)

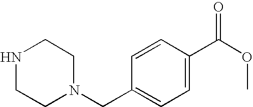

To a solution of 4-Formyl-benzoic acid methyl ester (0.167 g, 1.02 mmol.) and piperazine (0.557 g, 6.47 mmol.) in mixed solvent of MeOH (5 mL) and DCM (5 mL), was added Na₂BH₃CN (0.111 g, 1.76 mmol.) and followed by acetic acid (0.75 mL, 13.1 mmol.). After being stirred at room temperature for 1 h, the reaction mixture was basified with aqueous Na₂CO₃ and extracted with DCM (x2). After workup, the residue was purified by preparative reverse-phase HPLC and the title compound was obtained as 2*TFA salt (0.195 g, 42%). HPLC purity (254 nm)=98%; LC-MS (ESI, positive mode) m/z 235 ([M+H]⁺).

¹H NMR (CD₃OD) δ 8.01 (d, 2H, J=8.3 Hz), 7.58 (d, 2H, J=8.3 Hz), 4.39 (s, 2H), 3.84 (s, 3H, OCH₃), 3.52-3.47 (m, 8H); ¹³C NMR (CD₃OD) 167.7, 135.3, 132.4, 131.2, 61.1, 52.9, 49.5, 42.2.

### Intermediate 33

### Preparation of 3-(4-piperazin-1-ylmethyl-phenyl)-acrylic acid methyl ester

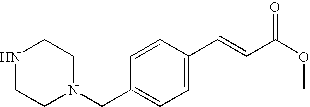

Proceeding as described in INTERMEDIATE 32 above but using appropriate starting material (3-(4-Formyl-phenyl)-acrylic acid methyl ester). LC-MS (ESI, positive mode) m/z 261 ([M+H]⁺).

### Intermediate 34

### Preparation of O-(2,4-Dimethoxy-benzyl)-hydroxylamine

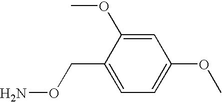

This compound was made according to the procedure described in the publication (Barlaam B., et al. Tetrahedron Lett. 39: 7865-7868 (1998)).

### Intermediate 35

### Preparation of 6-Amino-hexanoic acid (2,4-dimethoxy-benzyloxy)-amide

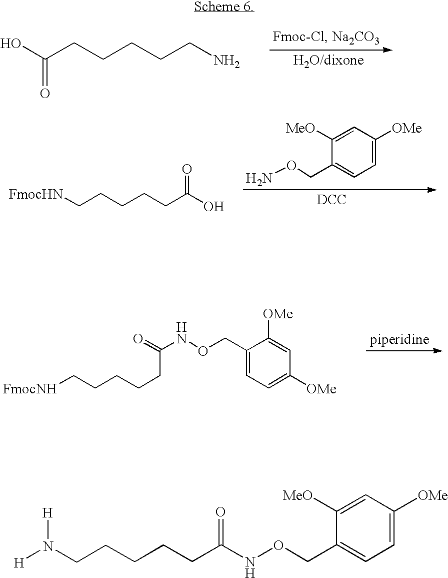

6-Amino-hexanoic acid (13.1 g, 100 mmol.) was dissolved in 10% aqueous Na₂CO₃ solution (300 mL), then dioxane (200 mL) was added to the above solution. Fmoc-Cl (26 g, 110 mmol.) was added to the above mixture portion-wise, and the resultant reaction mixture was stirring for 12 h. The mixture was extracted with ether (150 mL×2), and the aqueous portion was acidified by 6N HCl. The mixture was filtered, and the solid was washed with water and dried to give 6-(9H-Fluoren-9-ylmethoxycarbonylamino)-hexanoic acid as a white solid (31 g. 81%).

6-(9H-Fluoren-9-ylmethoxycarbonylamino)-hexanoic acid (9.17 g, 25 mmol.) and O-(2,4-dimethoxy-benzyl)-hydroxylamine (36 g, 26 mmol.) were dissolved in DCM (250 mL), then DCC (6.18 g, 30 mmol.) was added portion-wise. The resultant mixture was stirred for 3 h at room temperature, then cooled to 0° C., filtered, and washed with DCM. The organic solution was evaporated to dryness to give the crude [5-(2,4-dimethoxy-benzyloxycarbamoyl)-pentyl]-carbamic acid 9H-fluoren-9-ylmethyl ester.

The crude ester was reacted with piperidine (5 mL) in MeOH (150 mL) at room temperature for 12 h. The solution was evaporated and the residue was purified by flash chromatography (silica, EtOAc:MeOH=5:1). 6-Amino-hexanoic acid (2,4-dimethoxy-benzyloxy)-amide was obtained as a white solid (4.15 g, 56%).

### Intermediate 36

### Preparation of 7-Amino-heptanoic acid (2,4-dimethoxy-benzyloxy)-amide

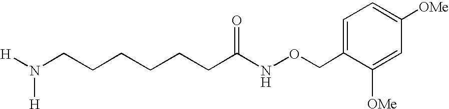

Proceeding as described in INTERMEDIATE 35 above but using appropriate starting materials.

### Intermediate 37

### Preparation of 8-Amino-octanoic acid (2,4-dimethoxy-benzyloxy)-amide

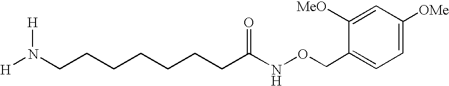

Proceeding as described in INTERMEDIATE 35 above but using appropriate starting materials.

### EXAMPLE 1

### Preparation of 6-[3-(toluene-4-sulfonyl)ureido]-hexanoic acid hydroxyamide

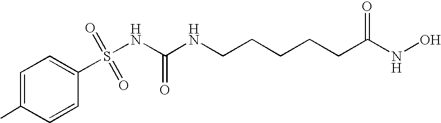

To a solution of 6-[3-(toluene-4-sulfonyl)ureido]-hexanoic acid methyl ester (0.035 g, 0.1 mmol) in dry MeOH (2 mL) was added NH₂OH.HCl (0.021 g, 0.3 mmol) followed by NaOMe (0.11 mL, 5.38M, 0.6 mmol). The reaction mixture was stirred at room temperature under nitrogen for 2 hours. The formation of the hydroxamic acid was followed by LCMS. Upon consumption of the starting material, the reaction mixture was diluted with acetonitrile and the solvent was removed in vacuo. The crude residue was purified by mass induced HPLC purification system to give 6-[3-(toluene-4-sulfonyl)ureido]-hexanoic acid hydroxyamide as a pale yellow/whitish solid.

¹H NMR (DMSO-d₆) δ 10.37 (1H, bs), 10.13 (1H, s), 8.46 (1H, s), 7.60 (2H, d, J=8.3 Hz, aromatic CH), 7.23 (2H, d, J=8.0 Hz, CH), 6.27 (1H, t, J=5.2 Hz), 2.92 (2H, q, J=6.1 Hz), 2.39 (3H, s), 1.89 (2H, t, J=7.5 Hz), 1.43 (2H, penta, J=7.5 Hz), 1.31 (2H, penta, J=7.4 Hz), 1.17-1.09 (2H, m).

### EXAMPLE 2

### Preparation of N-Hydroxy-{3-[4-[3-(toluene-4-sulfonyl)ureido]-phenyl}-acrylamide

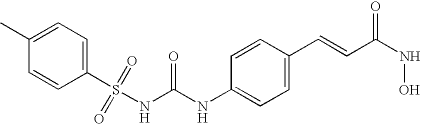

Proceeding as described in EXAMPLE 1 above but using appropriate starting materials

Yield: 5% from the corresponding methyl ester. White solid. HPLC purity at 254 nm: 93%; LC-MS (ESI, positive mode) m/z 376 ([M+H]⁺).

### EXAMPLE 3

### Preparation of N-Hydroxy-3-{3-[3-(4-methylbenzenesulfonyl)ureido]-phenyl}-acrylamide

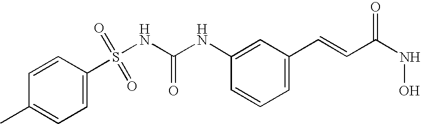

Proceeding as described in EXAMPLE 1 above but using appropriate starting materials.

Yield: 64%. White solid. HPLC purity at 254 nm: 95%. LC-MS (ESI, positive mode) m/z 376 ([M+H]⁺). ¹H NMR (DMSO-d₆) δ 7.70 (d, 2H, J=6.0 Hz), 7.36 (d, 2H, J=8.1 Hz), 7.30 (d, 1H, J=15.8 Hz), 7.25 (s, 1H), 7.11 (t, 1H, J=7.7 Hz), 6.80 (d, 1H, J=8.2 Hz), 6.68 (d, 1H, J=7.6 Hz), 6.33 (d, 1H, J=15.8 Hz), 2.37 (s, 3H, —CH₃).

### EXAMPLE 4

### Preparation of 4-[3-(toluene-4-sulfonyl)ureidomethyl-N-hydroxy-benzamide

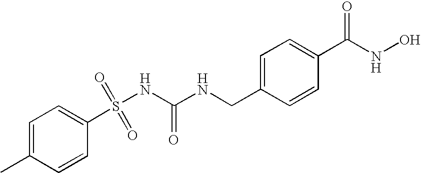

Proceeding as described in EXAMPLE 1 above but using appropriate starting materials.

Yield: 58%. White solid. HPLC purity at 254 nm: 100%. LC-MS (ESI, positive mode) m/z 364 ([M+H]⁺); ¹H NMR (DMSO-d₆) δ 11.14 (s, 1H), 10.74 (s, 1H), 8.98 (d, 1H, J=1.7 Hz), 7.79 (d, 2H, J=8.3), 7.65 (d, 2H, J=8.3 Hz), 7.41 (d, 2H, J=8.0 Hz), 7.18 (d, 2H, J=8.2 Hz), 7.05 (t, 1H, J=5.8 Hz), 4.19 (d, 2H, J=5.9), 2.47 (s, 3H).

### EXAMPLE 5

### Preparation of N-Hydroxy-2-{4-[3-(toluene-4-sulfonyl)ureido]-phenyl}acetamide

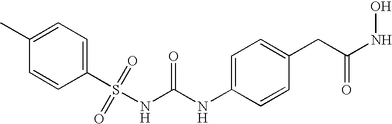

Proceeding as described in EXAMPLE 1 above but using appropriate starting materials.

Yield: 99%. White solid. HPLC purity at 254 nm: 99%.

LC-MS (ESI, positive mode) m/z 364 ([M+H]⁺);

¹H NMR (DMSO-d₆) δ10.47 (s, 1H), 8.64 (s, 1H), 7.73 (d, 2H, J=8.2 Hz), 7.32 (d, 2H, J=8.1 Hz), 7.13 (d, 2H, J=8.5 Hz), 7.02 (d, 2H, J=8.3 Hz), 3.08 (s, 2H), 2.29 (s, 2H); ¹³C NMR (DMSO-d₆) δ 167.0, 149.2, 141.9, 137.1, 130.8, 129.4, 129.2, 127.4, 125.6, 118.8, 38.6, 21.0.

### EXAMPLE 6

### Preparation of 4-(3-Benzoyl-ureidomethyl)-N-hydroxy-benzamide

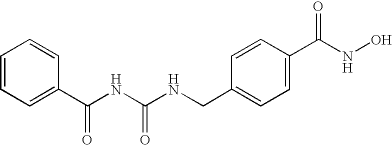

To a solution of 4-(3-Benzoyl-ureidomethyl)-benzoic acid methyl ester (0.030 g, 0.096 mmol) in dry MeOH (0.5 mL) was added NH₂OH.HCl (0.020 g, 0.288 mmol) followed by 30% NaOMe solution (5.38 M, 0.106 mL, 0.576 mmol). The reaction mixture was stirred at room temperature under nitrogen for 22 hours then quenched by addition of concentrated hydrochloric acid. The mixture was subjected to RPHPLC for purification. 4-(3-Benzoyl-ureidomethyl)-N-hydroxy-benzamide was obtained as white solid (yield 47%).

HPLC purity at 254 nm: 99.7%, t_{R}=4.55 min.

LC-MS (ESI, positive mode) m/z 314 ([M+H]⁺);

¹H NMR (DMSO-d₆) δ 11.11 (s, 1H), 10.71 (s, 1H), 9.04-9.07 (tr, 1H, J=6.0 Hz), 8.92 (br s, 1H), 7.89-7.91 (d, 2H, J=8.4 Hz), 7.65-7.67 (d, 2H, J=8.3 Hz), 7.54-7.58 (m, 1H), 7.42-7.48 (m, 2H), 7.32-7.34 (d, 2H, J=8.3 Hz), 4.42-4.43 (d, 2H, J=6.0 Hz), 2.47 (s, 3H); ¹³C NMR (DMSO-d₆) δ 167.5, 153.0, 141.8, 132.0, 131.8, 130.7, 128.4, 127.8, 127.7, 127.4, 126.3, 41.8. Anal. Calculated for C₁₆H₁₅N₃O₄: C, 61.34; H, 4.83; N, 13.41. Found: C, 61.31; H, 4.79; N, 13.38.

### EXAMPLE 7

### Preparation of 2-[3-(3-Benzoyl-ureido)-phenyl]-N-hydroxy-acetamide

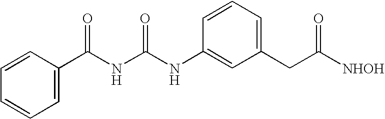

Prepared from the corresponding methyl ester. Yield: 7%. White solid. HPLC purity at 254 nm: 99%; LC-MS (ESI, positive mode) m/z 314 ([M+H]⁺);

¹H NMR (DMSO-d₆) δ 11.02 (s, 1H), 10.84 (s, 1H), 10.66 (s, 1H), 8.83 (s, 1H), 8.01-8.038.02 (d, 2H, J=8.5 Hz), 7.64-7.667.65 (m, 1H), 7.53-7.57 (m, 2H), 7.49-7.517.50 (dd, 1H, J=8.1 Hz), 7.44 (s, 1H), 7.27-7.30 (tr, 1H, J=7.8 Hz), 7.00-7.027.01 (d, 1H, J=7.8 Hz), 3.29 (s, 2H).

### EXAMPLE 8

### Preparation of 2-[4-(3-Benzoyl-ureido)-phenyl]-N-hydroxy-acetamide

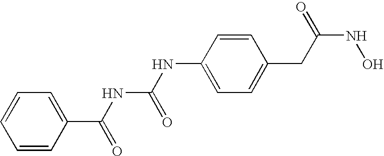

Prepared from the corresponding methyl ester. Yield: 2%. White solid. HPLC purity at 254 nm: 98%; LC-MS (ESI, positive mode) m/z 314 ([M+H]⁺).

### EXAMPLE 9

### Preparation of 3-{4-[3-Benzoyl-1-(3-hydroxy-propyl)-ureidomethyl]-phenyl}-N-hydroxy-acrylamide

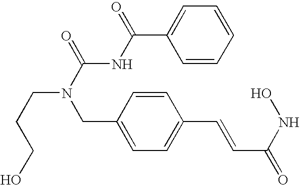

Proceeding as described in EXAMPLE 23 above but using appropriate starting materials the crude titled compound was purified by reverse-phase preparative HPLC.

HPLC purity at 254 nm: 97%; LC-MS (ESI, positive mode) m/z 398 ([M+H]⁺).

¹H NMR (DMSO-d₆) δ 10.69 (1H, s), 10.27 (1H, s), 8.97 (s, 1H), 7.76 (br d, 2H, J=6.4 Hz), 7.54 (t, 1H, J=7.3 Hz), 7.49 (d, 2H, J=8.4 Hz), 7.42 (t, 2H, J=7.6 Hz), 7.38 (d, 1H, J=15.9 Hz), 7.31 (br d like, 2H), 6.39 (d, 1H, J=15.8 Hz), 4.80 (br s, 1H), 4.51 (s, 2H), 3.25 and 3.32 (each 2H, overlapped with solvent peak), 1.61 (m, 2H).

### EXAMPLE 11

### Preparation of 6-(3-Benzoyl-ureido)-hexanoic acid hydroxyamide

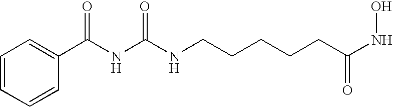

### Method A:

To a solution of 6-(3-Benzoyl-ureido)-hexanoic acid (0.0033 g, 0.01 mmol) in DMF (1 mL) was added Py-BOP (0.07 g, 0.013 mmol) and N,N-diisopropylethylamine (0.013 mL, 0.07 mmol). The reaction mixture was stirred for 5 minutes and the NH₂OH.HCl (0.02 g, 0.02 mmol) was added. The reaction mixture was stirred overnight at room temperature under nitrogen. The crude reaction mixture was purified by mass induced HPLC to give 6-(3-Benzoyl-ureido)-hexanoic acid hydroxyamide as an off white solid.

### Method B:

To a solution of 6-(3-Benzoyl-ureido)-hexanoic acid methyl ester (0.300 g, 1.03 mmol) in dry MeOH (2.0 mL) was added NH₂OH.HCl (0.555 g, 8.00 mmol) followed by 30% NaOMe in MeOH (2.23 mL, 5.38M, 12.0 mmol). The reaction mixture was stirred at room temperature under nitrogen for 1 h then was added trifluoroacetic acid (0.3 mL) in an ice-bath. The solution was extracted with 10% MeOH in dichloromethane. The extract was dried and concentrated. The residue was purified by reverse-phase preparative HPLC to give 6-(3-Benzoyl-ureido)-hexanoic acid hydroxyamide (0.175 g, 59%) as white solid. HPLC purity at 254 nm: 99.7%, t_{R}=5.15 min. LC-MS (ESI, positive mode) m/z 293 ([M+H]⁺).

¹H NMR (DMSO-d₆) δ 10.63 (1H, s), 10.34 (1H, s), 8.70-8.60 (1H, bs), 8.65 (1H, t, J=5.7 Hz), 7.95 (2H, dt, J=7.2, 1.6 Hz), 7.62 (1H, tt, J=7.4, 1.2 Hz), 7.50 (2H, t, J=7.9 Hz), 3.22 (2H, q, J=6.6 Hz, CH₂N), 1.96 (2H, t, J=7.4 Hz, CH₂CO), 1.56-1.46 (4H, m), 0.130-1.24 (2H, m); ¹³C NMR (DMSO-d₆) δ169.1 (CONHOH), 168.2 (PhCO), 153.4 (NHCONH), 132.7 (CH), 132.6 (Cq), 128.4 (CH×2), 128.0 (CH×2), 38.9 (CH₂N), 32.2 (CH₂CO), 28.9, 25.9, 24.8. Anal. Calculated for C₁₄H₁₉N₃O₄: C, 57.33; H, 6.53; N, 14.33. Found: C, 57.06; H, 6.32; N, 13.88.

### EXAMPLE 11

### Preparation of 3-(4-{3-Benzoyl-1-[2-(1H-indol-3-yl)-ethyl]-ureidomethyl}phenyl)-N-hydroxy-acrylamide

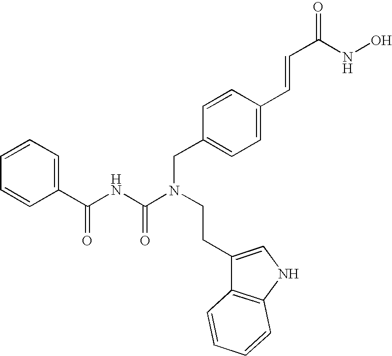

To a cooled solution of 3-(4-{3-Benzoyl-1-[2-(1H-indol-3-yl)-ethyl]-ureidomethyl}-phenyl)-acrylic acid methyl ester (crude, 2.86 g, 5.93 mmol.) and hydroxylamine hydrochloride (4.14 g, 59.5 mmol) in dry MeOH (40 mL) was added NaOMe in MeOH (4.37 M, 16.9 mL, 73.9 mmol) via syringe. The reaction mixture was stirred at room temperature under nitrogen for 1 h then was added dry ice powder, followed by addition of water and neutralized with 6N HCl to pH 6-7. The resultant mixture was concentrated to remove the organic solvent and the residue was filtered and washed with water. The residue was purified by preparative reverse-phase HPLC to give 3-(4-{3-Benzoyl-1-[2-(1H-indol-3-yl)-ethyl]-ureidomethyl}-phenyl)-N-hydroxy-acrylamide (0.85 g, 30%) as pale yellow or white solid. LC-MS (ESI, positive mode) m/z 483 ([M+H]⁺). HPLC purity (254 nm)=97%.

¹H NMR (DMSO-d₆) δ 10.80 (s, 1H), 10.77 (s, 1H), 10.28 (s, 1H), 9.05 (br s, 1H), 7.82 (d, 2H, J=7.4 Hz), 7.60 (t, 1H, J=7.3 Hz), 7.57 (d, 2H, J=8.3 Hz), 7.50 (t or d, 2H, J=7.7 Hz), 7.46 (d, 1H, J=14.2 Hz), 7.46-7.34 (br m, 3H), 7.10 (br s, 1H), 7.02 (t, 1H, J=7.4 Hz), 6.9-6.7 (very br s, 1H), 6.47 (d, 1H, J=15.8 Hz), 4.67 (s, 2H), 3.52 (dt or br t-like, 2H, J=6.7 Hz), 2.96 (br t-like, 2H, J=7.6 Hz); ¹³C NMR (DMSO-d₆) δ 166.5, 162.8, 154.1, 139.0, 138.0 (CH═), 136.1, 133.8, 133.2, 132.2, 128.4, 127.92, 127.88, 127.6, 126.9, 122.9, 120.9, 118.8, 118.2, 118.0, 111.4, 110.7, 49.4*, 49.2*, 24.5* (* these peaks are weak and broad, identified by ¹H-¹³C HSQC). Anal. Calculated for C₂₈H₂₆N₄O₄: C, 69.70; H, 5.43; N, 11.61. Found: C, 69.43; H, 5.45; N, 11.62.

### EXAMPLE 12

### Preparation of 8-(3-Benzoyl-ureido)-octanoic acid hydroxyamide

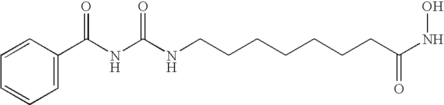

To a solution of 8-(3-Benzoyl-ureido)-octanoic acid methyl ester (0.275 g, equal to 0.811 mmol) and NH₂OH.HCl (0.562 g, 8.09 mmol) was added dry MeOH (5 mL) and followed by NaOMe in MeOH (2.30 mL, 4.37 M, 10.0 mmol). The reaction mixture was stirred at room temperature under nitrogen for 50 min then was neutralized with trifluoroacetic acid (0.80 mL). The mixture was purified by reverse-phase preparative HPLC (C₁₈, 5 um, 21.2×150 mm, 20 mL/min, 5 to 95% of CH₃CN+0.05% TFA over 18 min), to give 8-(3-Benzoyl-ureido)-octanoic acid hydroxyamide as white powder (0.115 g, 44%).

LC-MS (ESI, positive mode) m/z 322 ([M+H]⁺).

¹H NMR (DMSO-d₆) δ10.64 (1H, s), 10.34 (1H, s), 8.70-8.60 (1H, bs), 8.66 (1H, t, J=5.1 Hz), 7.96 (2H, d, J=7.5 Hz), 7.62 (1H, t, J=7.0 Hz), 7.50 (2H, t, J=7.3 Hz), 3.23 (2H, q, J=6.1 Hz, CH₂N), 1.95 (2H, t, J=7.2 Hz, CH₂CO), 1.50-1.48 (4H, m), 1.29-1.22 (6H, m); ¹³C NMR (DMSO-d₆) δ 169.1 (CONHOH), 168.2 (PhCO), 153.5 (NHCONH), 132.7 (CH), 132.6 (Cq), 128.4 (CH×2), 128.1 (CH×2), 39.0 (CH₂N), 32.2 (CH₂CO), 29.1, 28.5, 28.4, 26.3, 25.0.

### EXAMPLE 13

### Preparation of 7-(3-Benzoyl-ureido)-heptanoic acid hydroxyamide

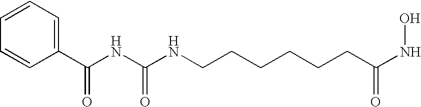

Proceeding as described in Example 12 above but using appropriate starting materials (7-(3-Benzoyl-ureido)-heptanoic acid methyl ester), and the reaction mixture was neutralized by TFA and evaporated to dryness. The residue was washed with water and the titled compound was obtained as white solid (0.175 g, 67% in two steps).

LC-MS (ESI, positive mode) m/z 308 ([M+H]⁺). HPLC purity at 254 nm: 98.7%

¹H NMR (DMSO-d₆) δ 10.64 (1H, s), 10.35 (1H, s), 8.70-8.60 (1H, bs), 8.64 (1H, t, J=5.6 Hz), 7.96 (2H, d, J=7.4 Hz), 7.62 (1H, t, J=7.4 Hz), 7.50 (2H, t, J=7.7 Hz), 3.22 (2H, q, J=6.5 Hz, CH₂N), 1.95 (2H, t, J=7.3 Hz, CH₂CO), 1.55-1.40 (4H, m), 1.35-1.20 (4H, m); ¹³C NMR (DMSO-d₆) δ 169.1 (CONHOH), 168.2 (PhCO), 153.4, 132.7, 132.6 (Cq), 128.4 (CH×2), 128.1 (CH×2), 39.0, 32.2, 29.1, 28.2, 26.1, 25.0.

### EXAMPLE 14

### 8-[3-(4-methylbenzenesulfonyl)-ureido])-octanoic acid hydroxyamide

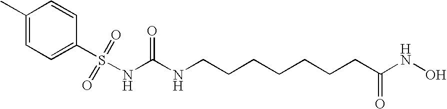

Proceeding as described in EXAMPLE 1 above but using appropriate starting materials. LC-MS (ESI, positive mode) m/z 372 ([M+H]⁺). HPLC purity at 254 nm: 100%.

### EXAMPLE 15

### 7-[3-(4-methylbenzenesulfonyl)-ureido])-heptanoic acid hydroxyamide

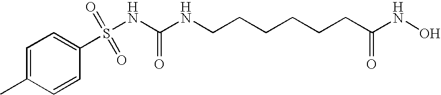

Proceeding as described in EXAMPLE 1 above but using appropriate starting materials. LC-MS (ESI, positive mode) m/z 358 ([M+H]⁺). HPLC purity at 254 nm: 100%.

### EXAMPLE 16

### 6-[3-(benzenesulfonyl)-ureido])-hexanoic acid hydroxyamide

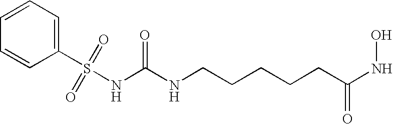

Proceeding as described in EXAMPLE 1 above but using appropriate starting materials. LC-MS (ESI, positive mode) m/z 330 ([M+H]⁺). HPLC purity at 254 nm: 100%.

### EXAMPLE 17

### Preparation of 6-[3-Benzoyl-1-(3-phenyl-propyl)-ureido]-hexanoic acid hydroxyamide

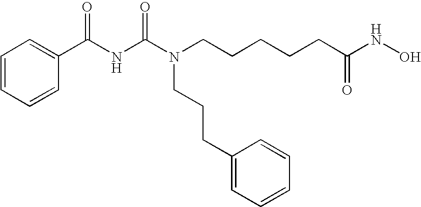

Proceeding as described in Example 12 above but using appropriate starting materials 6-[3-Benzoyl-1-(3-phenyl-propyl)-ureido]-hexanoic acid methyl ester, and the reaction mixture was neutralized by TFA and was purified by reverse-phase HPLC to give the titled compound as a gum (15 mg, 32%). LC-MS (ESI, positive mode) m/z 412 ([M+H]⁺). HPLC purity at 254 nm: 98.1%

¹H NMR (DMSO-d₆) δ 10.34 (1H, s), 10.08 (1H, s), 7.81 (2H, d, J=7.1 Hz), 7.59 (1H, t, J=7.4 Hz), 7.49 (2H, t, J=7.5 Hz), 7.30-7.10 (5H, m), 3.40-3.20 (4H, m), 2.57 (2H, m), 1.93 (2H, t or penta like, J=7.0 Hz), 1.85 (2H, penta, J=7.2 Hz), 1.60-1.40 (4H, m), 1.30-1.10 (2H, m); ¹³C NMR (DMSO-d₆) δ 169.0, 166.1, 153.5, 133.4, 132.0, 128.4, 128.2, 128.1 127.8, 125.7, 48.0*, 46.4*, 32.22, 32.17, 29.9*, 27.9*, 25.8, 24.8 (*: very weak and broad peaks identified by ¹H—¹³C HSQC).

### EXAMPLE 18

### Preparation of 6-(3-Benzoyl-thioureido)-hexanoic acid hydroxyamide

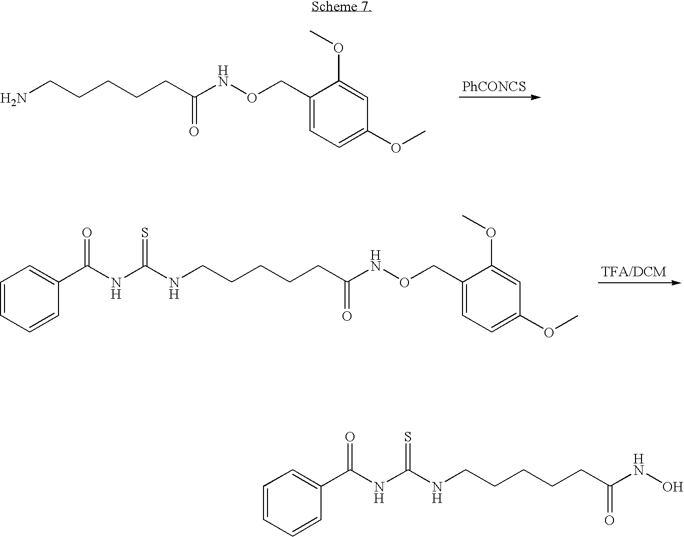

### Step 1

### Preparation of 6-(3-Benzoyl-thioureido)-hexanoic acid (2,4-dimethoxy-benzyloxy)-amide

To a solution of 6-Amino-hexanoic acid (2,4-dimethoxy-benzyloxy)-amide (0.160 mg, 0.54 mmol) in Dichloromethane (DCM, 4 mL) was added triethylamine (0.11 mL, 0.79 mmol) and Benzoyl isothiocyanate (0.11 mL, 0.82 mmol). The reaction was stirred at room temperature overnight and worked up. The residue (0.369 g) was used without further purification. LC-MS: m/z=460 (M+H).

### Step 2

### Preparation of 6-(3-Benzoyl-thioureido)-hexanoic acid hydroxyamide

To a solution of 6-(3-Benzoyl-thioureido)-hexanoic acid (2,4-dimethoxy-benzyloxy)-amide (crude from STEP 1, 0.186 g equal to 0.27 mmol.) and Triethylsilane (0.05 mL) in DCM (1.7 mL) was added TFA (0.3 mL) at room temperature with stirring. After 20 min, the solution was evaporated to dryness and diluted with methanol and filtered. The filtrate was concentrated and the residue was purified by preparative HPLC. 6-(3-Benzoyl-thioureido)-hexanoic acid hydroxyamide was obtained as a white solid (0.027 g, 32% overall yield). LC-MS (ESI, positive mode) m/z=310 (M+H). HPLC (254 nm) purity 95.4%. ¹H NMR (DMSO-d₆) δ 11.24 (s, 1H), 10.87 (s, 1H), 10.36 (s, 1H), 8.9-8.4 (very broad, 0.6H), 7.92 (d, 2H, J=7.4 Hz), 7.63 (t, 1H, J=7.4 Hz), 7.51 (t, 2H, J=7.7 Hz), 3.60 (dt or q-like, 2H, J=6.7 and 6.0 Hz), 1.97 (t, 2H, J=7.3 Hz), 1.54 (penta, 2H, J=6.0 Hz), 1.54 (penta, 2H, J=7.4 Hz), 1.32 (m, 2H); ¹³C NMR (DMSO-d₆) δ 179.9, 169.0, 168.0, 132.9, 132.2 (Cq), 128.45, 128.38, 44.6, 32.1, 27.3, 26.0, 24.8.

### EXAMPLE 19

### Preparation of 6-(3-Benzoyl-1-pyridin-2-ylmethyl-ureido)-hexanoic acid hydroxyamide

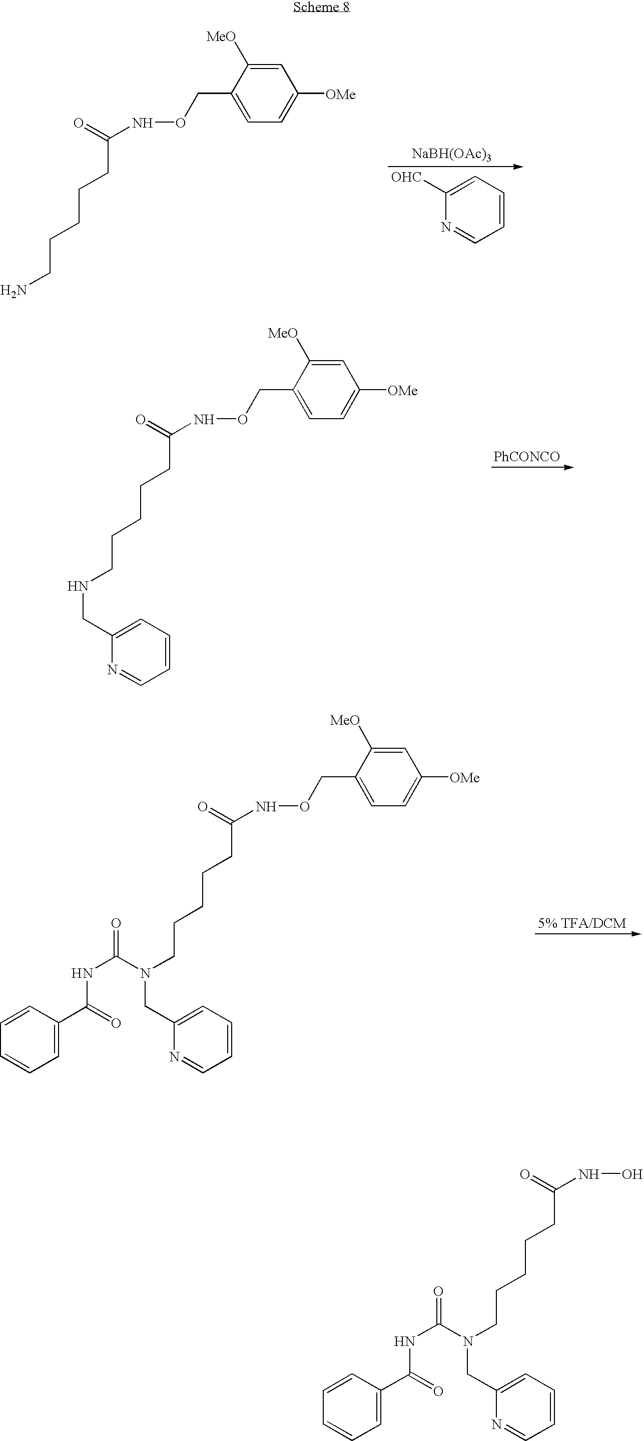

### Step 1

### Preparation of 6-[(Pyridin-2-ylmethyl)-amino]-hexanoic acid (2,4-dimethoxy-benzyloxy)-amide

By using analogous method described in INTERMEDIATE 13,6-Amino-hexanoic acid (2,4-dimethoxy-benzyloxy)-amide and pyridine-2-carbaldehyde was converted to 6-[(Pyridin-2-ylmethyl)-amino]-hexanoic acid (2,4-dimethoxy-benzyloxy)-amide.

### Step 2

### Preparation of 6-(3-Benzoyl-1-pyridin-2-ylmethyl-ureido)-hexanoic acid (2,4-dimethoxy-benzyloxy)-amide

By using analogous method described in INTERMEDIATE 14, 6-[(Pyridin-2-ylmethyl)-amino]-hexanoic acid (2,4-dimethoxy-benzyloxy)-amide was converted to the title compound.

### Step 3

### Preparation of 6-(3-Benzoyl-1-pyridin-2-ylmethyl-ureido)-hexanoic acid hydroxyamide

By using analogous method described in Example 18, Step 2,6-(3-Benzoyl-1-pyridin-2-ylmethyl-ureido)-hexanoic acid (2,4-dimethoxy-benzyloxy)-amide was deprotected by 15% TFA in DCM and provided 6-(3-Benzoyl-1-pyridin-2-ylmethyl-ureido)-hexanoic acid hydroxyamide as a TFA salt after preparative HPLC purification.

HPLC purity at 254 nm: 100%; LC-MS (ESI, positive mode) m/z 385.43 ([M+H]⁺); ¹H NMR (CD₃OD) δ 8.63-7.80 (br, m, Ar—H), 7.58-7.44 (m, Ar—H), 4.71-4.54 (d, 2H, N—CH₂-py), 3.44-3.40 (t, 2H, N—CH₂), 2.02-1.98 (t, 2H, O═C—CH₂), 1.61-1.50, 1.27-1.21 (m, 8H, CH₂); ¹³C NMR (CD₃OD) δ 170.8, 167.0, 154.4 (C═O), 148.7, 136.8 (Ar—C), 132.0, 127.9, 127.7, 127.5, 127.2, 127.1, 126.7, 12.4, 123.1, 122.1 (Ar—CH), 49.8, 31.6, 31.3, 26.6, 25.1, 24.8, 24.3, 24.0 (CH₂).

### EXAMPLE 20

### Preparation of 7-(3-Benzoyl-1-pyridin-2-ylmethyl-ureido)-heptanoic acid hydroxyamide

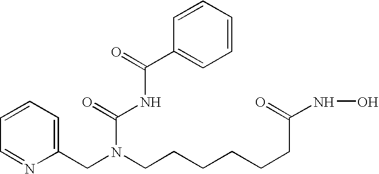

Proceeding as described in EXAMPLE 19 above but using appropriate starting materials the crude titled compound was purified by reverse-phase preparative HPLC and obtained as TFA salt. HPLC purity at 254 nm: 100%; LC-MS (ESI, positive mode) m/z 399 ([M+H]⁺); ¹H NMR (CD₃OD) δ 8.58-7.75 (br, m), 7.58-7.34 (m, Ar—H), 3.40-3.36 (t, 2H), 1.97-1.93 (t, 2H), 1.52-1.19 (br, m, 8H); ¹³C NMR (CD₃OD) δ131.9, 127.9, 127.1, 31.6, 27.6, 25.2, 24.5.

### EXAMPLE 21

### Preparation of 7-(3-Benzoyl-1-benzyl-ureido)-heptanoic acid hydroxyamide

Proceeding as described in EXAMPLE 19 above but using appropriate starting materials the crude titled compound was purified by reverse-phase preparative HPLC.

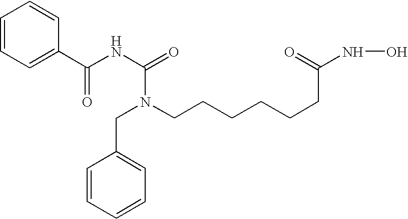

HPLC purity at 254 nm: 95%; LC-MS (ESI, positive mode) m/z 398 ([M+H]⁺); ¹H NMR (CD₃OD) δ 7.69-7.17 (br, m, Ar—H), 4.56 (s, 2H), 3.31-3.26 (t, 2H), 1.97-1.93 (t, 2H), 1.51-1.21 (m, 8H); ¹³C NMR (CD₃OD) δ170.9, 167.2, 154.2, 136.3, 132.7, 131.7, 128.9, 128.4, 127.8, 127.7, 127.2, 127.0, 126.7, 31.6, 27.7, 27.4, 26.4, 25.3, 24.5, 24.2.

### EXAMPLE 22

### Preparation of 6-(3-Benzoyl-1-benzyl-ureido)-hexanoic acid hydroxyamide

Proceeding as described in EXAMPLE 19 above but using appropriate starting materials the crude titled compound was purified by reverse-phase preparative HPLC.

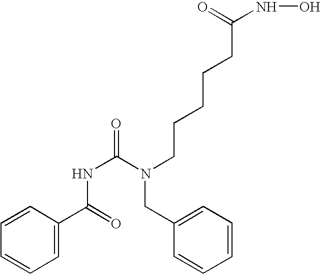

HPLC purity at 254 nm: 96%; LC-MS (ESI, positive mode) m/z 384 ([M+H]⁺); ¹H NMR (CD₃OD) δ 7.68-7.16 (br, m, Ar—H), 4.52 (s, 2H), 3.31-3.27 (t, 2H), 1.98-1.95 (t, 2H), 1.55-1.22 (m, 7H, CH₂); ¹³C NMR (CD₃OD) δ 136.3, 132.7 (Ar—C), 127.8, 127.7, 127.0, 126.7, 31.6, 25.2, 24.3 (CH₂).

### EXAMPLE 23

### Preparation of 3-[4-(3-Benzoyl-1-phenethyl-ureidomethyl)-phenyl]-N-hydroxy-acrylamide

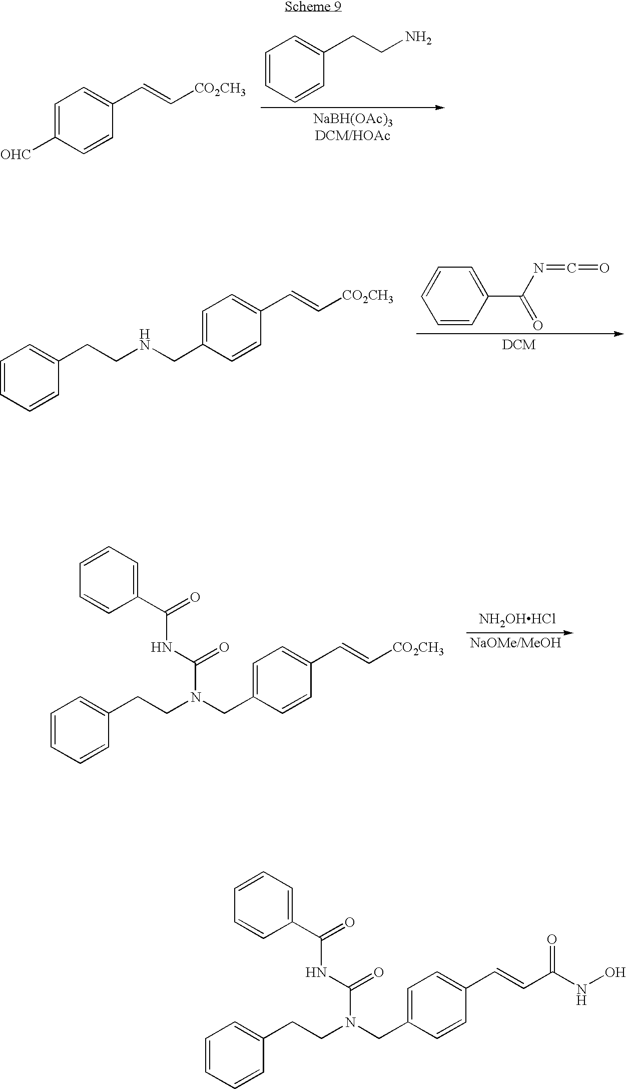

### Step 1

### Preparation of 3-[4-(Phenethylamino-methyl)-phenyl]-acrylic acid methyl ester

To a solution of 3-(4-Formyl-phenyl)-acrylic acid methyl ester (4.16 g, 5.17 mmol) in DCM (150 mL) was added phenethylamine (4.05 g, 33.4 mmol) and the solution was stirred at room temperature for 1 hour. NaBH(OAc)₃ (9.15 g, 38.9 mmol) was added to the above solution in portions and followed by acetic acid (2 mL, 34.9 mmol) an dth emixture was stirred at room temperature overnight. The mixture was basified by adding aqueous NaHCO₃ and extracted with EtOAc (x3). After workup, the residue was purified by flash chromatography (silica, EtOAc:DCM:MeOH=100:95:5) and provided 3-[4-(Phenethylamino-methyl)-phenyl]-acrylic acid methyl ester as white solid (5.47 g, 86%). LC-MS (ESI, positive mode) m/z=296 (M+H)

### Step 2

### Preparation of 3-[4-(3-Benzoyl-1-phenethyl-ureidomethyl)-Phenyl]-acrylic acid methyl ester

To a mixture of 3-[4-(Phenethylamino-methyl)-phenyl]-acrylic acid methyl ester (3.01 g, 10.2 mmol.) and benzoylisocyante (2.43 g, 90% pure, 14.8 mmol.) was added DCM (30 mL) and followed by Et₃N (1.8 mL, 12.9 mmol.). The solution was stirred at room temperature overnight and evaporated to dryness. The crude solid product can be used for next step of reaction without further purification. LC-MS (ESI, positive mode) m/z=443 (M+H).

### Step 3

### Preparation of 3-[4-(3-Benzoyl-1-phenethyl-ureidomethyl)-phenyl]-N-hydroxy-acrylamide

To a cooled solution of 3-[4-(3-Benzoyl-1-phenethyl-ureidomethyl)-phenyl]-acrylic acid methyl ester (crude from STEP 2, 10.2 mmol.) and hydroxylamine hydrochloride (7.08 g, 102 mmol.) in MeOH (60 mL) was slowly added sodium methoxide solution in MeOH (4.37 M, 30 mL, 131 mmol.) via a syringe. The resultant mixture was then stirred at room temperature for about 2 h (monitoring the progress by LC-MS) and quenched by adding dry-ice powder. The cold mixture was added de-ionized water and pH was adjusted to 3-4 by adding of 4N HCl. The solution was evaporated to remove all the organic solvent and the residue was washed with water (x3). The crude product was purified by preparative reverse phase HPLC and provided 3-[4-(3-Benzoyl-1-phenethyl-ureidomethyl)-phenyl]-N-hydroxy-acrylamide as white powder/solid (0.945 g, 21% from STEP 2). HPLC (254 nm) purity 97%. LC-MS (ESI, positive mode) m/z=444 (M+H).

¹H NMR (DMSO-d₆) δ 10.76 (s, 1H), 10.27 (s, 1H), 9.04 (br s, 1H), 7.81 (d, 2H, j=7.3 Hz), 7.60 (t, 1H, J=7.3 Hz), 7.56 (d, 2H, J=8.0 Hz), 7.50 (d or t, 2H, J=7.2 Hz), 7.44 (d, 1H, J=16.4 Hz), 7.39 (br d, 2H), 7.26 (t, 2H, J=7.2 Hz), 7.21-7.10 (m, 3H), 6.46 (1H, d, J=15.8 Hz), 4.60 (s, 2H), 3.48 (t, 2H, J=7.7 Hz), 2.84 (t, 2H, J=7.6 Hz); ¹³C NMR (DMSO-d₆) δ 166.6, 162.8, 154.0, 139.0, 138.7, 138.0, 133.8, 133.4, 132.2, 128.6, 128.42, 128.40, 127.90, 127.88, 127.6, 126.3, 118.9, 49.7*, 49.5*, 34.2* (* these peaks are weak and broad, identified by ¹H-¹³C HSQC). Anal. Calculated for C₂₆H₂SN₃O₄: C, 70.41, H, 5.68; N, 9.47. Found: C, 69.95, H, 5.97; N, 9.41.

### EXAMPLE 24

### Preparation of 3-{4-[3-Benzoyl-1-(2-morpholin-4-yl-ethyl)-ureidomethyl]-phenyl}-N-hydroxy-acrylamide

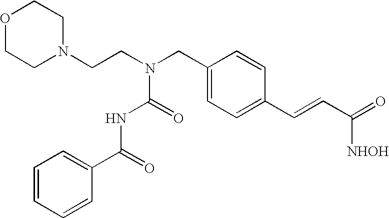

Proceeding as described in EXAMPLE 23 above but using appropriate starting materials the crude titled compound was purified by reverse-phase preparative HPLC as TFA salt. LC-MS (ESI, positive mode) m/z 453 ([M+H]⁺); ¹H NMR (CD₃OD) δ 7.71-7.25 (br, m, Ar—H), 6.40-6.36 (d, 1H, J=16 Hz), 4.64 (s, 2H), 3.82-3.71 (br, m, 5H), 3.32-3.25 (br, t, 2H); ¹³C NMR (CD₃OD) δ167.7, 164.1, 155.1, 137.1, 134.2 (Ar—C), 138.8, 132.2, 132.1, 127.8, 127.5, 127.2, 117.0, 63.1, 54.2, 51.9, 41.2.

### EXAMPLE 25

### Preparation of 3-(4-{3-Benzoyl-1-[2-(4-bromo-phenyl)-ethyl]-ureidomethyl}-phenyl)-N-hydroxy-acrylamide

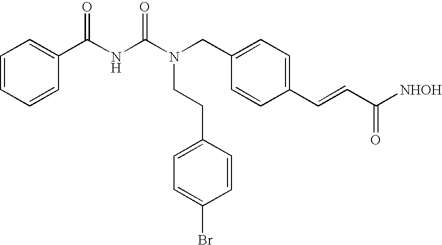

Proceeding as described in EXAMPLE 23 above but using appropriate starting materials the crude titled compound was purified by reverse-phase preparative HPLC.

HPLC purity at 254 nm: 96%; LC-MS (ESI, positive mode) m/z 524 ([M+H]⁺); ¹H NMR (CD₃OD) δ 7.67-7.01 (br, m, Ar—H), 6.41-6.37 (d, 1H, J=16 Hz), 4.56 (s, 2H), 3.54-3.50 (br, t, 2H), 2.81-2.77 (br, t, 2H); ¹³C NMR (CD₃OD) δ169.1, 141.0, 140.0, 134.5, 133.7, 138.3, 132.7, 131.9, 131.7, 131.5, 129.6, 129.4, 129.1, 128.9, 121.3 (CH═CH), 34.6.

### EXAMPLE 26

### Preparation of 3-(4-{3-Benzoyl-1-[2-(4-fluoro-phenyl)-ethyl]-ureidomethyl}-phenyl)-N-hydroxy-acrylamide

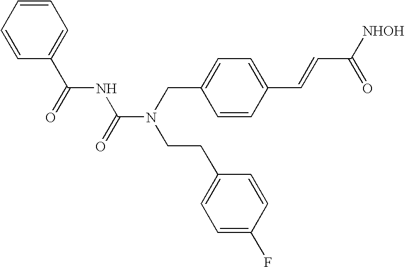

Proceeding as described in EXAMPLE 23 above but using appropriate starting materials the crude titled compound was purified by reverse-phase preparative HPLC.

HPLC purity at 254 nm: 91%; LC-MS (ESI, positive mode) m/z 462 ([M+H]⁺); ¹H NMR (CD₃OD) δ 7.68-7.66 (br, m, Ar—H), 6.41-6.37 (d, 1H, J=16 Hz), 4.56 (s, 2H), 3.52-3.26 (br t, 2H), 2.82-2.79 (br t, 2H); ¹³C NMR (CD₃OD) δ 167.2, 162.4, 159.9, 138.2, 134.0, 132.6, 116.6, 139.1, 131.8, 129.7, 129.6, 114.4, 114.2, 116.6, 32.4.

### EXAMPLE 27

### Preparation of 3-{4-[3-Benzoyl-1-(3-imidazol-1-yl-propyl)-ureidomethyl]-phenyl}-N-hydroxy-acrylamide

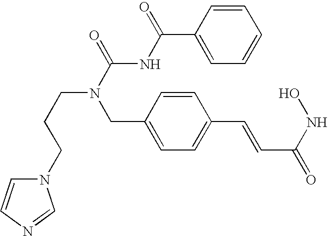

Proceeding as described in EXAMPLE 23 above but using appropriate starting materials the crude titled compound was purified by reverse-phase preparative HPLC as TFA salt. HPLC purity at 254 nm: 98%; LC-MS (ESI, positive mode) m/z 448 ([M+H]⁺); ¹H NMR (CD₃OD) δ 8.83 (s, 1H, NH), 7.70-7.26 (b, m, Ar—H), 6.40-6.36 (d, 1H, J=16 Hz), 4.24 (br s, 2H), 3.40-3.37 (br t, 2H), 2.17-2.10 (br, t, 2H); ¹³C NMR (CD₃OD) δ138.9, 132.0, 136.2, 127.8, 127.3, 127.1, 121.2, 119.2, 116.9, 45.9, 27.15.

### EXAMPLE 28

### Preparation of 3-(4-{3-Benzoyl-1-[2-(1H-imidazol-4-yl)-ethyl]-ureidomethyl}-phenyl)-N-hydroxy-acrylamide

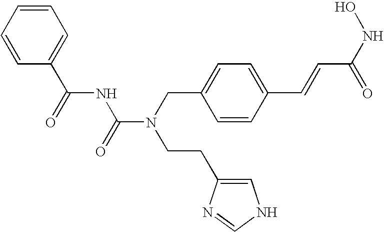

Proceeding as described in EXAMPLE 23 above but using appropriate starting materials the crude titled compound was purified by reverse-phase preparative HPLC as TFA salt. HPLC purity at 254 nm: 100%; LC-MS (ESI, positive mode) m/z 434 ([M+H]⁺); ¹H NMR (CD₃OD) δ 8.67 (s, 1H, NH), 7.72-7.24 (b, m, Ar—H), 6.44-6.40 (d, 1H, J=16 Hz), 4.57 (br, s, 2H), 3.71-3.68 (br, t, 2H), 3.02-2.99 (br, t, 2H); ¹³C NMR (CD₃OD) δ 167.3, 164.3, 154.4, 137.9, 133.9, 132.3, 130.2, 139.0, 132.3, 131.9, 127.8, 127.3, 127.1, 116.8, 116.1 (CH═CH), 45.9, 22.0.

### EXAMPLE 29

### Preparation of 3-{4-[1-(1H-Benzoimidazol-2-ylmethyl)-3-benzoyl-ureidomethyl]-phenyl}-N-hydroxy-acrylamide

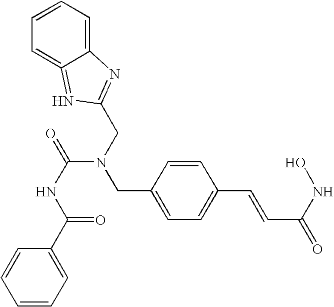

Proceeding as described in EXAMPLE 23 above but using appropriate starting materials the crude titled compound was purified by reverse-phase preparative HPLC.

LC-MS (ESI, positive mode) m/z 470 ([M+H]⁺); ¹H NMR (CD₃OD) δ 7.80-7.31 (br, m, Ar—H), 6.41-6.37 (d, 1H, J=16 Hz), 4.81 (s, 2H), 4.70 (s, 2H); ¹³C NMR (CD₃OD) δ 166.9, 154.1, 134.0, 133.5, 137.8, 132.4, 128.4, 128.1, 127.7, 124.2, 119.0, 114.5.

### EXAMPLE 30

### Preparation of 4-(4-Benzoylaminocarbonyl-piperazin-1-ylmethyl)-N-hydroxy-benzamide

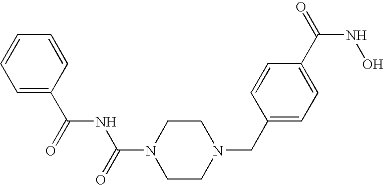

Proceeding as described in EXAMPLE 23 (STEP 2 and 3) above but using appropriate starting material (INTERMEDIATE 32). The crude titled compound was purified by reverse-phase preparative HPLC and obtained as TFA salt. LC-MS (ESI, positive mode) m/z 383 ([M+H]⁺). ¹H NMR (CD₃OD) δ 7.83 (d, 2H, J=7.2 Hz), 7.63 (d, 2H, J=7.7 Hz), 7.56 (t, 1H, J=7.5 Hz), 7.53 (d, 1H, J=15.7 Hz), 7.51 (d, 2H, J=6.4 Hz), 7.45 (t, 2H, J=7.5 Hz), 4.38 (s, 2H), 4.0˜3.2 (very br m, 8H).

### EXAMPLE 31

### Preparation of N-{4-[4-(2-Hydroxycarbamoyl-vinyl)-benzyl]-piperazine-1-carbonyl}-benzamide

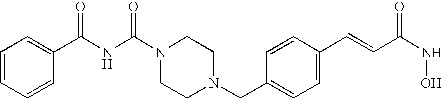

Proceeding as described in EXAMPLE 23 (STEP 2 and 3) above but using appropriate starting material (INTERMEDIATE 33). The crude titled compound was purified by reverse-phase preparative HPLC and obtained as freebase after basification (23% from two steps). LC-MS (ESI, positive mode) m/z 408 ([M+H]⁺). HPLC purity (254 nm)=94%.

¹H NMR (CD₃OD) δ7.83 (d, 2H, J=7.3 Hz), 7.80 (d, 2H, J=8.3 Hz), 7.58 (d, 2H, J=8.1 Hz), 7.55 (t, 1H, J=7.4 Hz), 7.44 (t, 2H, J=7.6 Hz)., 4.42 (s, 2H), 4.3˜3.3 (very br m, 8H).

### EXAMPLE 32

### Preparation of 6-[2-(3-Benzoyl-ureido)-3-(1H-indol-3-yl)-propionylamino]-hexanoic acid hydroxyamide

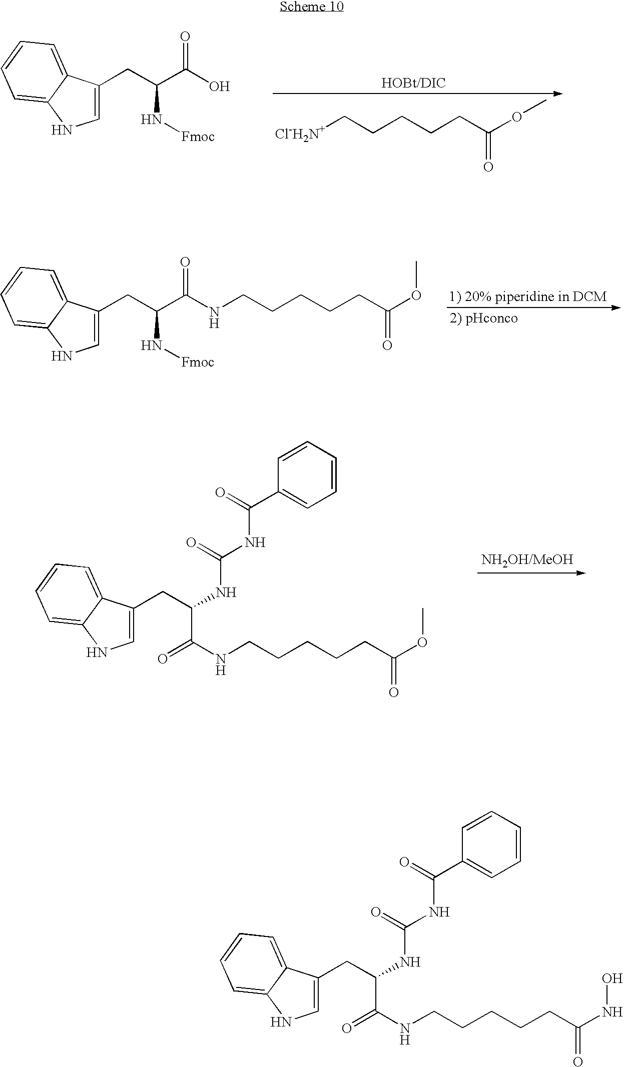

### Step 1

### Preparation of (S)-6-[2-(9H-Fluoren-9-ylmethoxycarbonylamino)-3-(1H-indol-3-yl)-propionylamino]-hexanoic acid methyl ester

To a solution of Fmoc-L-Tryptophan (0.422 g, 0.99 mmol) and HOBt hydrate (0.171 g, 1.13 mmol) in Dichloromethane (DCM, 10 mL) was added diisopropyl-carbodiimide (DIC, 0.170 mL, 1.09 mmol.). After being stirred at room temperature for 1 h, 6-Amino-hexanoic acid methyl ester hydrochloride salt (0.201 g, 1.11 mmol.) was added to the above solution and followed by diisopropylethylamine (0.210 mL, 1.21 mmol.). The reaction mixture was stirred overnight, worked up and purified by flash chromatography (silica, 50% to 100% of EtOAc in hexanes). LC-MS (ESI, positive mode) m/z=554 (M+H).

### Step 2

### Preparation of 6-[2-Amino-3-(1H-indol-3-yl)-propionylamino]-hexanoic acid methyl ester

To a solution of (S)-6-[2-(9H-Fluoren-9-ylmethoxycarbonylamino)-3-(1H-indol-3-yl)-propionylamino]-hexanoic acid methyl ester (crude, 0.433 g, equal to 0.61 mmol.) in DCM (4 mL) was added piperidine (1 mL). After being stirred at room temperature for 30 min, the solution was evaporated to dryness and the residue was washed with hexanes (x4) and worked up to give the title compound (0.219 g). LC-MS (ESI, positive mode) m/z=332 (M+H).

Proceeding as described in EXAMPLE 23 (STEP 2 and 3) above but using appropriate starting material (6-[2-Amino-3-(1H-indol-3-yl)-propionylamino]-hexanoic acid methyl ester). The crude titled compound was purified by reverse-phase preparative HPLC. LC-MS (ESI, positive mode) m/z 480 ([M+H]⁺). HPLC purity (254 nm)=94%.

¹H NMR (CD₃OD) δ 7.79 (d or dd, 2H, J=7.2, 1.3 Hz), 7.53 (d, 1H, J=5.4 Hz), 7.51 (t, 1H, J=7.5 Hz), 7.40 (t, 2H, J=7.7 Hz), 7.23 (d, 1H, J=8.1 Hz), 7.09 (s, 1H), 6.98 (td, 1H, J=7.0, 0.9 Hz), 6.90 (td, 1H, J=7.4, 0.7 Hz), 4.51 (t, 1H, J=6.7 Hz), 3.18 (2H, overlapped by HDO), 3.03 and 2.91 (m, each 1H), 1.93 (t, 2H, J=7.4 Hz), 1.42 (penta, 2H, J=7.5 Hz), 1.20 (penta, 2H, J=7.5 Hz), 1.04 (m, 2H).

### EXAMPLE 33

### Preparation of 3-[4-(3-Benzoyl-1-pyridin-3-ylmethyl-ureidomethyl)-phenyl]-N-hydroxy-acrylamide

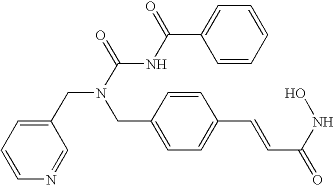

Proceeding as described in EXAMPLE 23 above but using appropriate starting materials the crude titled compound was purified by reverse-phase preparative HPLC.

HPLC purity at 254 nm: 99%; LC-MS (ESI, positive mode) m/z 431 ([M+H]⁺); ¹H NMR (DMSO-d₆) δ 10.70 (br s, 1H), 10.40 (s, 1H), 8.52* (d like, 2H, including 1H (d, J=4.0 Hz) and 1H (s)), 7.87 (br, 1H), 7.74 (d, 2H, J=7.3 Hz), 7.51 (t, 1H, J=7.4 Hz), 7.49 (overlapped, 1H), 7.48*(d, 2H, J=8.0 Hz), 7.42 (t, 2H, J=7.8 Hz), 7.38 (d, 1H, J=16.7 Hz), 7.26* (d, 2H, J=7.3 Hz), 6.39 (d, 1H, J=15.8 Hz), 4.56 (s, 2H), 4.54 (s, 2H); ¹³C NMR (DMSO-d₆) δ 166.6, 162.5*, 154.5, 146.4*(br, CH×2), 138.3, 137.8, 137.5*, 134.0, 133.1, 132.3, 128.4, 127.9* (CH×2×2), 124.4, 119.0, 51.9*, 48.3* (* these peaks are identified by ¹H-¹³C HSQC and HMBC).

### EXAMPLE 34

### Preparation of 4-(3-Benzoyl-ureido)-N-hydroxy-butyramide

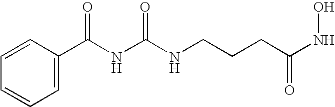

Proceeding as described in EXAMPLE 10 above but using appropriate starting materials the crude titled compound was purified by reverse-phase preparative HPLC.

HPLC purity at 254 nm: 98%; LC-MS (ESI, positive mode) m/z 266 ([M+H]⁺); ¹H NMR (DMSO-d₆) δ 10.60 (s, 1H), 10.34 (s, 1H), 8.62 (br, 1H), 8.61 (t, 1H, J=5.7 Hz), 7.89 (d, 2H, J=7.4 Hz), 7.55 (t, 1H, J=7.4 Hz), 7.44 (t, 2H, J=7.7 Hz), 3.16 (q-like, 2H, J=6.4 Hz), 1.94 (t, 2H, J=7.5 Hz), 1.66 (penta, 2H, J=7.3 Hz); ¹³C NMR (DMSO-d₆) δ 168.6, 168.1, 153.5, 132.7, 132.6, 128.5, 128.1, 38.7, 29.8, 25.5.

### EXAMPLE 35

### Preparation of 3-{4-[3-Benzoyl-1-(3-phenyl-propyl)-ureidomethyl]-phenyl}-N-hydroxy-acrylamide

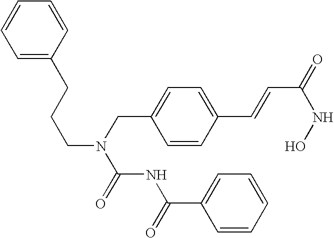

Proceeding as described in EXAMPLE 23 above but using appropriate starting materials the crude titled compound was purified by reverse-phase preparative HPLC.

LC-MS (ESI, positive mode) m/z 458 ([M+H]⁺); ¹H NMR (CD₃OD) δ 7.65 (d, 2H, J=7.3 Hz), 7.49 (1H, t, J=7.4 Hz), 7.47 (1H, d, J=15.6 Hz), 7.44 (d, 2H, j=7.7 Hz), 7.37 (t, 2H, J=7.7 Hz), 7.27 (br d, 2H), 7.14 (t-like, 1H), 7.05 (m, 2H), 7.02 (m, 2H), 6.37 (d, 1H, J=15.8 Hz), 4.58 (s, 2H), 3.28 (m, 2H), 3.15 (s-like, 2H), 1.83 (penta, 2H, J=7.4 Hz).

### EXAMPLE 36

### Preparation of 3-{4-[3-Benzoyl-1-(2-phenoxy-ethyl)-ureidomethyl]-phenyl}-N-hydroxy-acrylamide

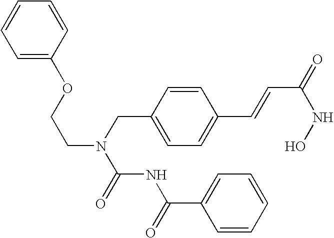

Proceeding as described in EXAMPLE 23 above but using appropriate starting materials the crude titled compound was purified by reverse-phase preparative HPLC.

LC-MS (ESI, positive mode) m/z 460 ([M+H]⁺); ¹H NMR (CD₃OD) δ 7.74 (br s, 2H), 7.50 (t, 1H, J=7.3 Hz), 7.45 (d, 1H, J=15.3 Hz), 7.43 (d, 2H, J=7.3 Hz), 7.37 (t, 2H, J=7.6 Hz), 7.32 (d, 2H, J=7.7 Hz), 7.14 (td, 2H, J=7.4, 1.2, Hz), 6.85 (t, 1H, J=7.4 Hz), 6.75 (d, 2H, J=7.4 Hz), 6.35 (d, 1H, J=15.8 Hz), 4.69 (s, 2H), 4.08 (t, 2H, J=4.8 Hz), 3.75 (t, 2H, J=4.9 Hz).

### EXAMPLE 37

### Preparation of 4-[3-Benzoyl-1-(3-phenyl-propyl)-ureidomethyl]-N-hydroxy-benzamide

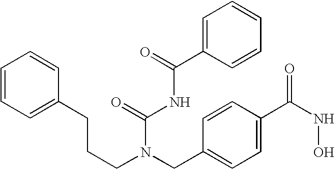

Proceeding as described in EXAMPLE 23 above but using appropriate starting materials the crude titled compound was purified by reverse-phase preparative HPLC.

LC-MS (ESI, positive mode) m/z 432 ([M+H]⁺); ¹H NMR (CD₃OD) δ 7.65 (d, 2H, J=7.6 Hz), 7.63 (d, 2H, J=8.3 Hz), 7.50 (t, J=7.3 Hz), 7.38 (t, 2H, J=7.7 Hz), 7.33 (br d, 2H, J=6.6 Hz), 7.10˜6.88 (m, 5H), 4.62 (s, 2H), 3.27 (m, 2H), 2.50 (t-like, 2H, J=6.9 Hz), 1.84 (penta, 2H, J=7.5 Hz).

### EXAMPLE 38

### Preparation of 4-(3-Benzoyl-1-pyridin-3-ylmethyl-ureidomethyl)-N-hydroxy-benzamide

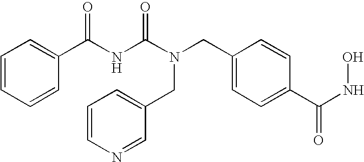

Proceeding as described in EXAMPLE 23 above but using appropriate starting materials the crude titled compound was purified by reverse-phase preparative HPLC.

HPLC purity at 254 nm: 98%; LC-MS (ESI, positive mode) m/z 405 ([M+H]⁺); ¹H NMR (CD₃OD) δ 8.73 (s, 1H), 8.64 (d, 1H, J=5.4 Hz), 8.38 (br d, 1H, J=6.9 Hz), 7.85 (t, 1H, J=6.8 Hz), 7.76 (d, 2H, J=8.5 Hz), 7.67 (d, 2H, J=8.3 Hz), 7.55 (tt, 1H, J=7.7 Hz), 7.43 (t, 2H, j=7.7 Hz), 7.34 (d, 2H, J=8.2 Hz), 4.79 (s, 2H), 4.73 (s, 2H).

### EXAMPLE 39

### Preparation of 4-(3-Benzoyl-1-benzyl-ureidomethyl)-N-hydroxy-benzamide

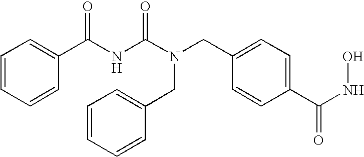

Proceeding as described in EXAMPLE 23 above but using appropriate starting materials the crude titled compound was purified by reverse-phase preparative HPLC.

HPLC purity at 254 nm: 97%; LC-MS (ESI, positive mode) m/z 404 ([M+H]⁺); ¹H NMR (CD₃OD) 7.638 (d, 2h, J=7.0 Hz), 7.636 (d, 2h, J=8.4 Hz), 7.48 (tt, 1H, j=7.4, 1.2 Hz), 7.36 (t, 2H, J=7.7 Hz), 7.31 (br d-like, 2H), 7.28˜7.17 (m, 5H), 4.58 (s, 2H), 4.53 (s, 2H).

### EXAMPLE 40

### Preparation of 4-[3-Benzoyl-1-(2-pyridin-2-yl-ethyl)-ureidomethyl]-N-hydroxy-benzamide

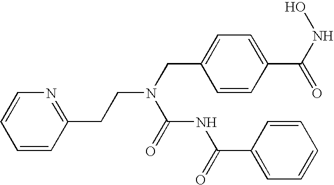

Proceeding as described in EXAMPLE 23 above but using appropriate starting materials the crude titled compound was purified by reverse-phase preparative HPLC.

LC-MS (ESI, positive mode) m/z 419 ([M+H]⁺); ¹H NMR (CD₃OD) δ 8.54 (br s, 1H), 8.28 (br s, 1H), 7.80˜7.60 (m, 6H), 7.48 (t, 1H, J=7.4 Hz), 7.37 (t, 2H, J=8.0 Hz), 7.30 (d, 2H, J=7.6 Hz), 4.62 (s, 2H), 3.82 (t, J=6.5 Hz), 3.25 (t, 2H, J=6.4 Hz).

### EXAMPLE 41

### Preparation of 4-[3-Benzoyl-1-(3-hydroxy-propyl)-ureidomethyl]-N-hydroxy-benzamide

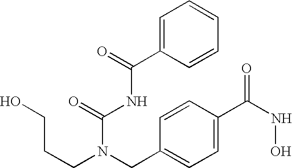

Proceeding as described in EXAMPLE 23 above but using appropriate starting materials the crude titled compound was purified by reverse-phase preparative HPLC.

HPLC purity at 254 nm: 97%; LC-MS (ESI, positive mode) m/z 372 ([M+H]⁺); ¹H NMR (CD₃OD) δ 7.78 (br m, 2H), 7.65 (d, 2H, J=8.3 Hz), 7.49 (t, 1H, J=7.4 Hz), 7.41-7.36 (m, 4H), 4.59 (s, 2H), 3.52 (t, 2H, J=5.8 Hz), 3.48 (t, 2H, J=6.5 Hz), 1.72 (t, 2H, J=6.0 Hz).

### EXAMPLE 42

### Preparation of 3-[4-(3-Benzoyl-1-benzyl-ureidomethyl)-phenyl]-N-hydroxy-acrylamide

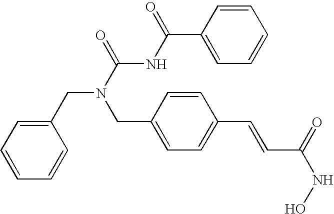

Proceeding as described in EXAMPLE 23 above but using appropriate starting materials the crude titled compound was purified by reverse-phase preparative HPLC.

HPLC purity at 254 nm: 96%; LC-MS (ESI, positive mode) m/z 430 ([M+H]⁺); ¹H NMR (CD₃OD) δ 7.67 (dd, 2H, J=8.5, 1.3 Hz), 7.55˜7.46 (m, 4H), 7.39 (t, 2H, J=7.4 Hz), 7.32-7.22 (m, 7H), 6.37 (d, 1H, J=15.7 Hz), 4.58 (s, 2H), 4.57 (s, 2H).

### EXAMPLE 43

### Preparation of 3-{4-[3-Benzoyl-1-(2-pyridin-2-yl-ethyl)-ureidomethyl]-phenyl}-N-hydroxy-acrylamide

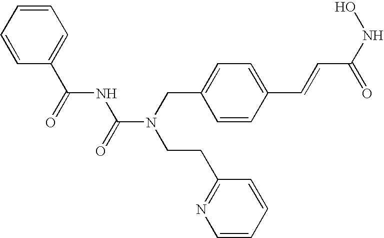

Proceeding as described in EXAMPLE 23 above but using appropriate starting materials the crude titled compound was purified by reverse-phase preparative HPLC.

HPLC purity at 254 nm: 96%; LC-MS (ESI, positive mode) m/z 558 ([M+H]⁺); ¹H NMR (CD₃OD) δ 8.56 (1H, s), 8.32 (br s, 1H), 7.83 (br s, 1H), 7.76 (br s, 1H), 7.66 (d, 2H, J=6.6 Hz), 7.51-7.45 (m, 4H), 7.37 (t, 2H, J=7.5 Hz), 7.25 (d, 2H, J=7.4 Hz), 6.39 (d, 1H, J=14.7 Hz), 4.58 (s, 2H), 3.82 (t, 2H, J=8.4 Hz), 3.26 (t, 2H, J=6.3 Hz).

### EXAMPLE 44

### Preparation of 3-{4-[3-Benzoyl-1-(2-pyridin-3-yl-ethyl)-ureidomethyl]-phenyl}-N-hydroxy-acrylamide

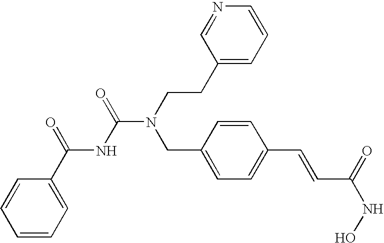

Proceeding as described in EXAMPLE 23 above but using appropriate starting materials the crude titled compound was purified by reverse-phase preparative HPLC.

LC-MS (ESI, positive mode) m/z 445 ([M+H]⁺); ¹H NMR (CD₃OD) δ8.67-8.39, 7.27 (br m, py-H), 7.84-7.35 (br, m, Ar—H), 6.40-6.36 (d, 1H, J=16 Hz), 4.58 (s, 2H), 3.73-3.69 (t, 2H), 3.10-3.07 (t, 2H); ¹³C NMR (CD₃OD) δ 167.1, 164.2, 154.4, 137.9, 133.9, 132.3 (Ar—C), 141.8, 138.9, 131.9, 127.8, 127.3, 127.2, 127.0, 126.0, 116.9, 29.8.

### EXAMPLE 45

### Preparation of 3-{4-[3-Benzoyl-1-(2-pyridin-4-yl-ethyl)-ureidomethyl]-phenyl}-N-hydroxy-acrylamide

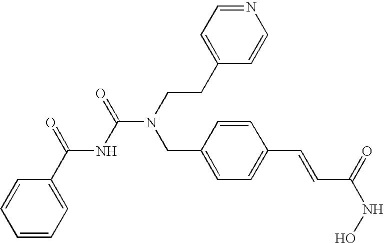

Proceeding as described in EXAMPLE 23 above but using appropriate starting materials the crude titled compound was purified by reverse-phase preparative HPLC.

HPLC purity at 254 nm: 100%; LC-MS (ESI, positive mode) m/z 445 ([M+H]⁺); ¹H NMR (CD₃OD) δ 8.66˜8.53 and 7.28-7.27 (br m), 7.86-7.35 (br m), 6.41-6.37 (d, 1H, J=16 Hz), 4.61 (s, 2H), 3.77-3.74 (t, 2H), 3.16-3.15 (t, 2H); ¹³C NMR (CD₃OD) δ 167.1, 164.2, 160.3, 154.4, 138.9, 137.8, 133.9, 132.3, 140.5, 131.9, 127.8, 127.3, 127.3, 127.2, 127.1, 116.9, 33.3.

### EXAMPLE 46

### Preparation of 3-{4-[3-Benzoyl-1-(2-piperidin-1-yl-ethyl)-ureidomethyl]-phenyl}-N-hydroxy-acrylamide

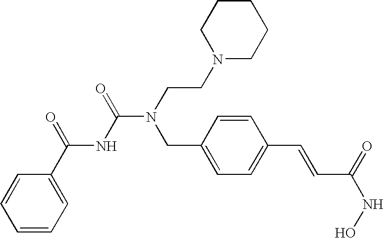

Proceeding as described in EXAMPLE 23 above but using appropriate starting materials the crude titled compound was purified by reverse-phase preparative HPLC.

HPLC purity at 254 nm: 100%; LC-MS (ESI, positive mode) m/z 451 ([M+H]⁺); ¹H NMR (CD₃OD) δ 7.70-7.32 (m, 11H), 6.43 (d, 1H, J=16 Hz), 4.61 (t, 2H), 3.77 (t, 2H), 3.56-3.53 (br, 2H), 2.94 (br, 2H), 1.90-1.47 (br, 6H); ¹³C NMR (CD₃OD) δ 167.4, 164.1, 137.2, 134.2, 132.1, 138.8, 132.2, 127.8, 127.5, 127.2, 117.0, 53.7, 53.1, 51.0, 41.6, 22.3, 20.6.

### EXAMPLE 47

### Preparation of 3-{4-[3-Benzoyl-1-(2-pyrrolidin-1-yl-ethyl)-ureidomethyl]-phenyl}-N-hydroxy-acrylamide

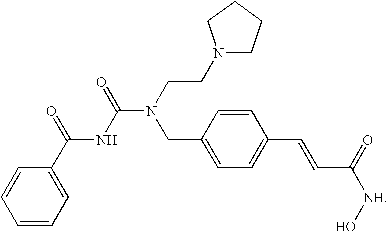

Proceeding as described in EXAMPLE 23 above but using appropriate starting materials the crude titled compound was purified by reverse-phase preparative HPLC.

HPLC purity at 254 nm: 97%; LC-MS (ESI, positive mode) m/z 437 ([M+H]⁺); ¹H NMR (CD₃OD) δ 7.70 (d, 2H, J=7.5 Hz), 7.52 (t, 1H, J=7.4 Hz), 7.51 (d, 2H, J=7.8 Hz), 7.48 (d, 1H, J=18.3 Hz), 7.39 (t, 2H, J=7.6 Hz), 7.30 (d, 2H, J=7.9 Hz), 6.40 (d, 1H, J=15.8 Hz), 4.66 (s, 2H), 3.72 (t, 2H, J=6.3 Hz), 3.67 (br m, 2H), 3.34 (t, 2H, J=6.2 Hz) N—CH₂), 3.05 (br m, 2H), 2.07 (br m, 2H), 1.96 (br m, 2H).

### Solid-Phase Synthesis of Acylurea Containing Hydroxamates.

The following protocol was used for synthesis of acylurea on solid-phase.

### Step 1.

The O-(2,4-Dimethoxy-benzyl)-hydroxylamine was attached to the aldehyde of SASRIN (Super Acid Sensitive Resin, Katritzky, A. R. 38: 7849-7850 (1997)) by reductive amination to give the protected acid labile hydroxylamine resin.

### Step 2.

3-(4-Formyl-phenyl)-acrylic acid was attached to the resin by treating with PyBroP (Bromo-tris-pyrrolidino-phosphonium hexafluorophosphate) and N,N-diisopropylethylamine (DIEA).

### Step 3.

Reductive amination with selected variety of amines.

### Step 4.

acylurea formation by reacting the above resin with benzoyl isocyanate.

### Step 5.

TFA cleavage and subsequent workup.

### Step 6.

The crude products were purified by High throughput mass-dependent HPLC purification system.

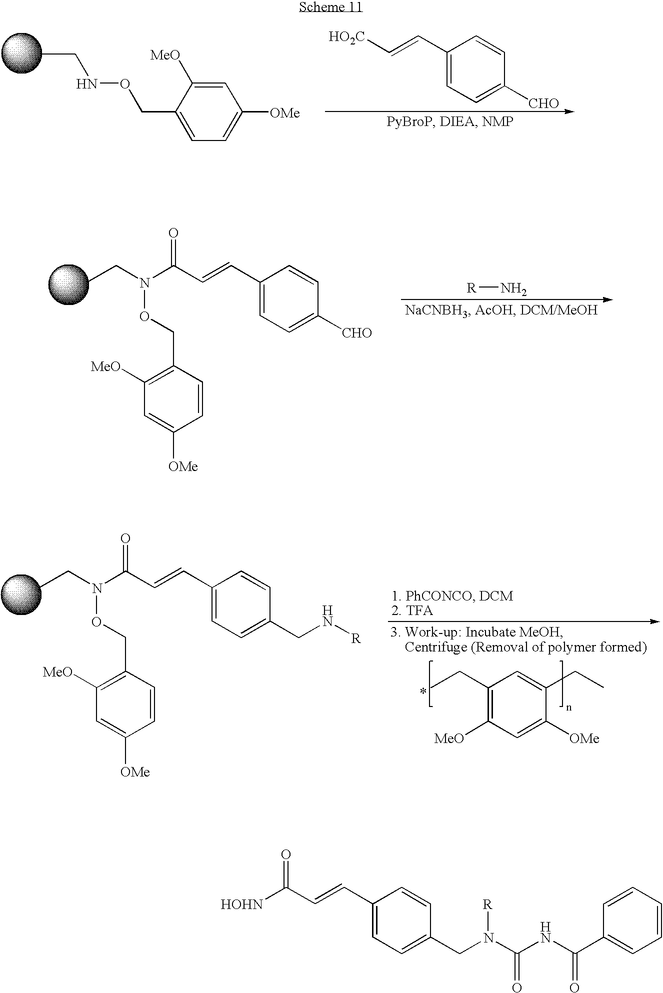

TABLE 1

| Compound | Structure | M + H | Name |
|---|---|---|---|
| L01 | 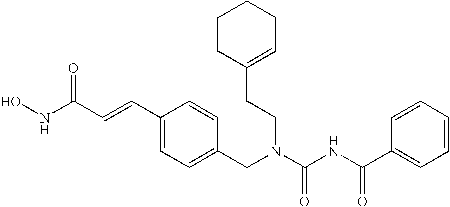 | 448 | 3-{4-[3-Benzoyl-1-(2-cyclohex-1-enyl-ethyl)-ureidomethyl]-phenyl}-N-hydroxy-acrylamide |
| L02 | 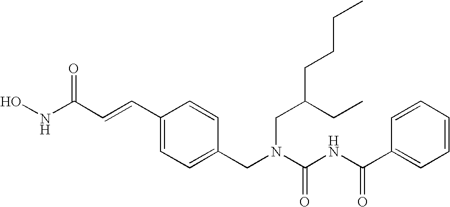 | 451 | 3-{4-[3-Benzoyl-1-(2-ethyl-hexyl)-ureidomethyl]-phenyl}-N-hydroxy-acrylamide |
| L03 | 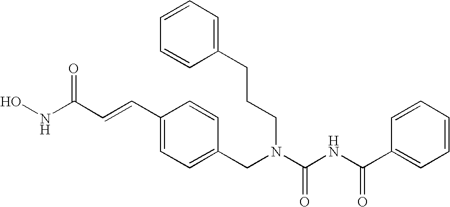 | 458 | 3-{4-[3-Benzoyl-1-(3-phenyl-propyl)-ureidomethyl]-phenyl}-N-hydroxy-acrylamide |
| L04 | 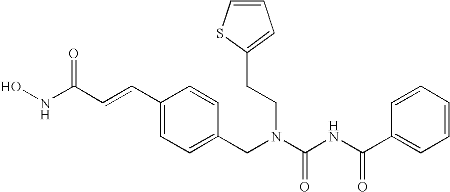 | 450 | 3-{4-[3-Benzoyl-1-(2-thiophen-2-yl-ethyl)-ureidomethyl]-phenyl}-N-hydroxy-acrylamide |
| L05 | 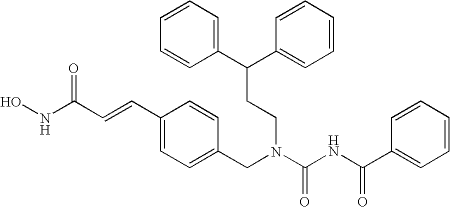 | 534 | 3-{4-[3-Benzoyl-1-(3,3-diphenyl-propyl)-ureidomethyl]-phenyl}-N-hydroxy-acrylamide |
| L06 | 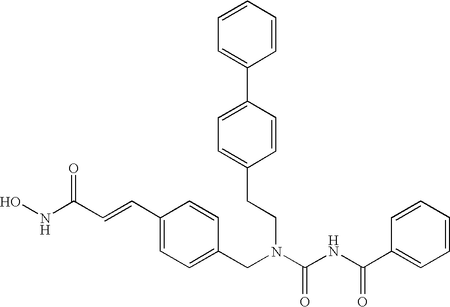 | 520 | 3-{4-[3-Benzoyl-1-(2-biphenyl-4-yl-ethyl)-ureidomethyl]-phenyl}-N-hydroxy-acrylamide |
| L07 | 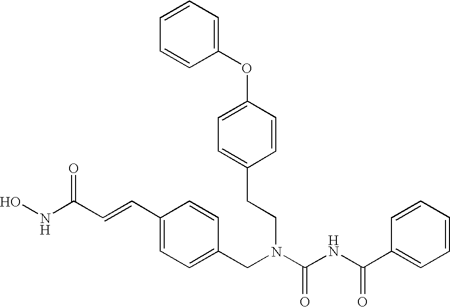 | 536 | 3-(4-{3-Benzoyl-1-[2-(4-phenoxy-phenyl)-ethyl]-ureidomethyl}-phenyl)-N-hydroxy-acrylamide |
| L08 | 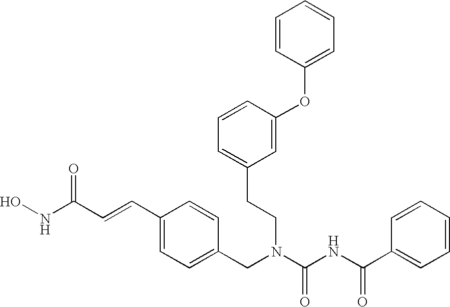 | 536 | 3-(4-{3-Benzoyl-1-[2-(3-phenoxy-phenyl)-ethyl]-ureidomethyl}-phenyl)-N-hydroxy-acrylamide |
| L09 | 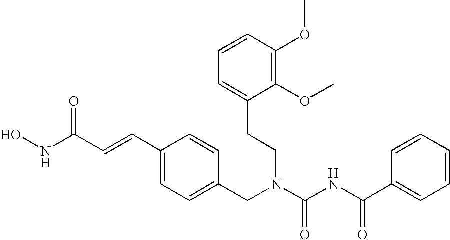 | 504 | 3-(4-{3-Benzoyl-1-[2-(2,3-dimethoxy-phenyl)-ethyl]-ureidomethyl}-phenyl)-N-hydroxy-acrylamide |
| L10 | 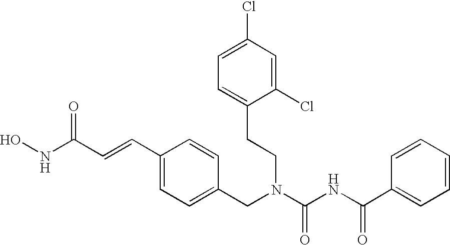 | 512 | 3-(4-{3-Benzoyl-1-[2-(2,4-dichloro-phenyl)-ethyl]-ureidomethyl}-phenyl)-N-hydroxy-acrylamide |
| L11 | 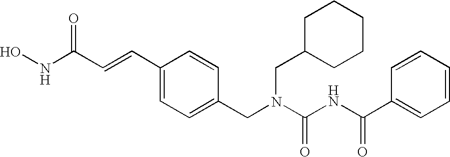 | 436 | 3-[4-(3-Benzoyl-1-cyclohexylmethyl-ureidomethyl)-phenyl]-N-hydroxy-acrylamide |
| L12 | 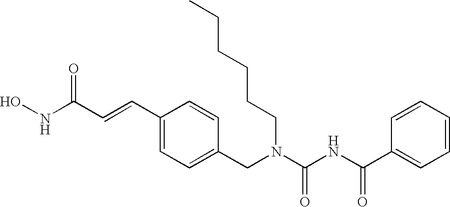 | 424 | 3-[4-(3-Benzoyl-1-hexyl-ureidomethyl)-phenyl]-N-hydroxy-acrylamide |
| L13 | 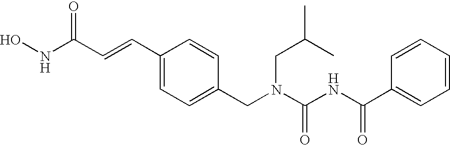 | 396 | 3-[4-(3-benzoyl-1-isobutyl-ureidomethyl)-phenyl]-N-hydroxy-acrylamide |
| L14 | 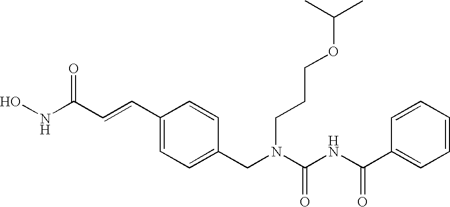 | 440 | 3-{4-[3-Benzoyl-1-(3-isopropoxy-propyl)-ureidomethyl]-phenyl}-N-hydroxy-acrylamide |
| L15 | 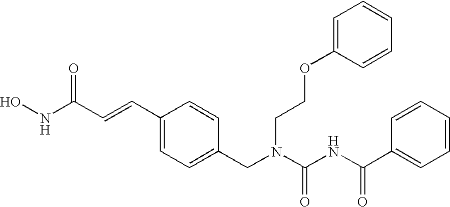 | 460 | 3-{4-[3-Benzoyl-1-(2-phenoxy-ethyl)-ureidomethyl]-phenyl}-N-hydroxy-acrylamide |
| L16 | 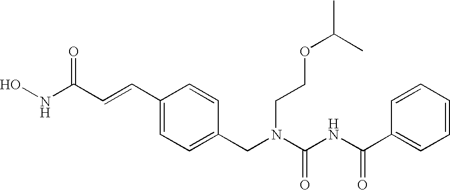 | 426 | 3-{4-[3-Benzoyl-1-(2-isopropoxy-ethyl)-ureidomethyl]-phenyl}-N-hydroxy-acrylamide |
| L17 | 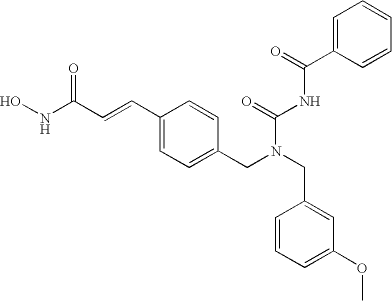 | 460 | 3-{4-[3-Benzoyl-1-(3-methoxy-benzyl)-ureidomethyl]-phenyl}-N-hydroxy-acrylamide |
| L18 | 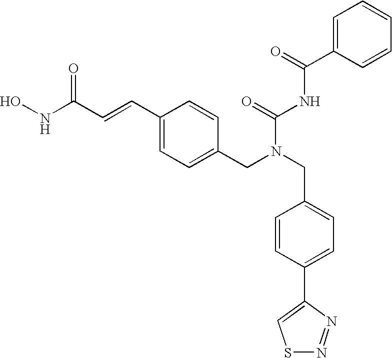 | 514 | 3-{4-[3-Benzoyl-1-(4-[1,2,3]thiadiazol-4-yl-benzyl)-ureidomethyl]-phenyl}-N-hydroxy-acrylamide |
| L19 | 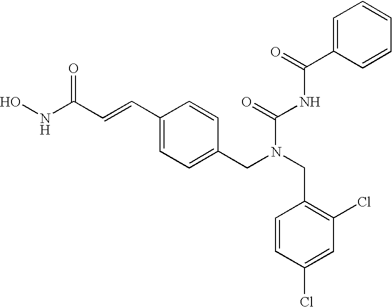 | 498 | 3-{4-[3-Benzoyl-1-(2,4-dichloro-benzyl)-ureidomethyl]-phenyl}-N-hydroxy-acrylamide |
| L20 | 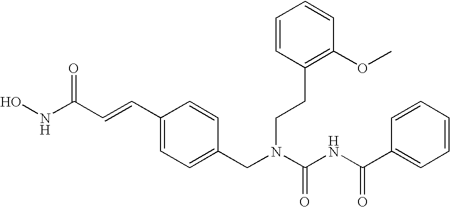 | 474 | 3-(4-{3-Benzoyl-1-[2-(2-methoxy-phenyl)-ethyl]-ureidomethyl}-phenyl)-N-hydroxy-acrylamide |
| L21 | 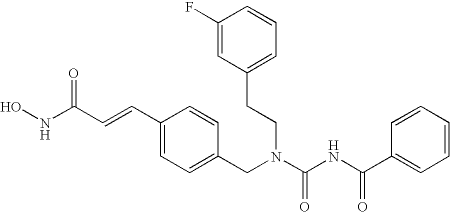 | 462 | 3-(4-{3-Benzoyl-1-[2-(3-fluoro-phenyl)-ethyl]-ureidomethyl}-phenyl)-N-hydroxy-acrylamide |
| L22 | 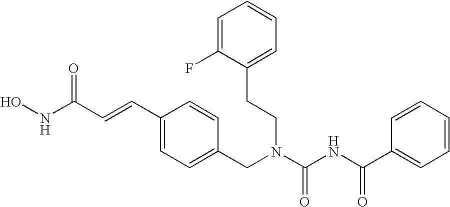 | 462 | 3-(4-{3-Benzoyl-1-[2-(2-fluoro-phenyl)-ethyl]-ureidomethyl}-phenyl)-N-hydroxy-acrylamide |
| L23 | 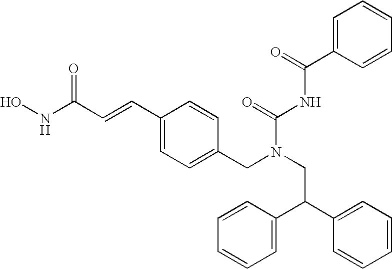 | 520 | 3-{4-[3-Benzoyl-1-(2,2-diphenyl-ethyl)-ureidomethyl]-phenyl}-N-hydroxy-acrylamide |
| L24 | 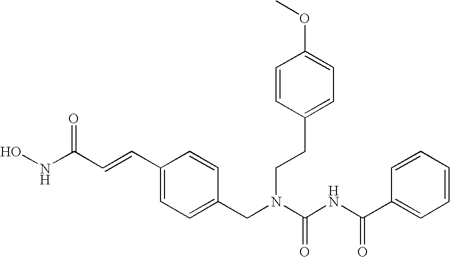 | 474 | 3-(4-{3-Benzoyl-1-[2-(4-methoxy-phenyl)-ethyl]-ureidomethyl}-phenyl)-N-hydroxy-acrylamide |
| L25 | 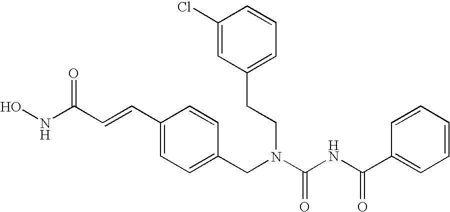 | 478 | 3-(4-{3-Benzoyl-1-[2-(3-chloro-phenyl)-ethyl]-ureidomethyl}-phenyl)-N-hydroxy-acrylamide |
| L26 | 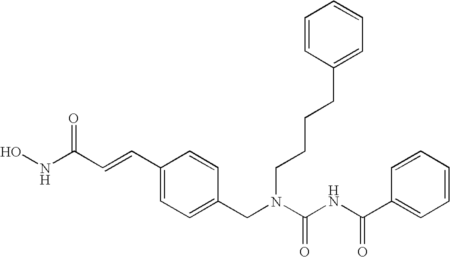 | 472 | 3-{4-[3-Benzoyl-1-(4-phenyl-butyl)-ureidomethyl]-phenyl}-N-hydroxy-acrylamide |
| L27 | 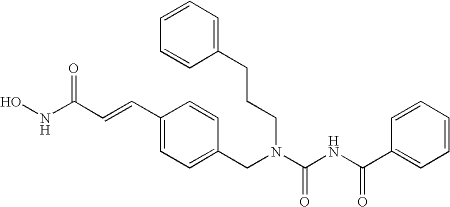 | 458 | 3-{4-[3-Benzoyl-1-(3-phenyl-propyl)-ureidomethyl]-phenyl}-N-hydroxy-acrylamide |
| L28 | 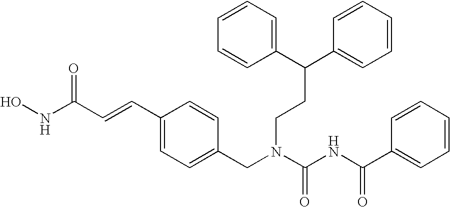 | 534 | 3-{4-[3-Benzoyl-1-(3,3-diphenyl-propyl)-ureidomethyl]-phenyl}-N-hydroxy-acrylamide |
| L29 | 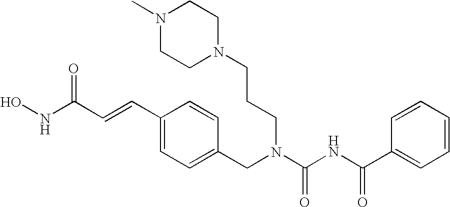 | 480 | 3-(4-{3-Benzoyl-1-[3-(4-methyl-piperazin-1-yl)-propyl]-ureidomethyl}-phenyl)-N-hydroxy-acrylamide |
| L30 | 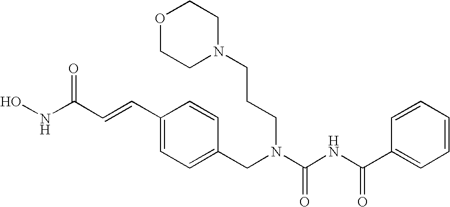 | 467 | 3-{4-[3-Benzoyl-1-(3-morpholin-4-yl-propyl)-ureidomethyl]-phenyl}-N-hydroxy-acrylamide |
| L31 | 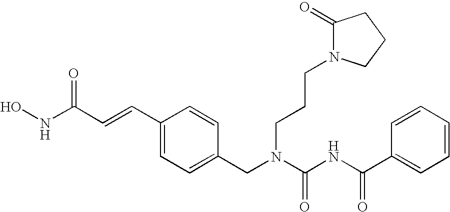 | 465 | 3-(4-{3-Benzoyl-1-[3-(2-oxo-pyrrolidin-1-yl)propyl]-ureidomethyl}-phenyl)-N-hydroxy-acrylamide |
| L32 | 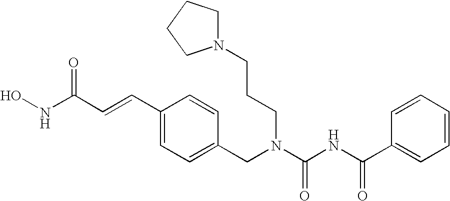 | 451 | 3-{4-[3-Benzoyl-1-(3-pyrrolidin-1-yl-propyl)-ureidomethyl]-phenyl}-N-hydroxy-acrylamide |
| L33 | 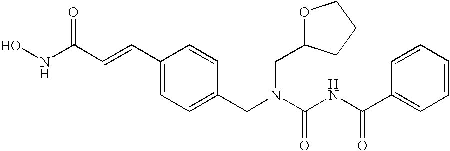 | 424 | 3-{4-[3-Benzoyl-1-(tetrahydro-furan-2-ylmethyl)-ureidomethyl]-phenyl}-N-hydroxy-acrylamide |
| L34 | 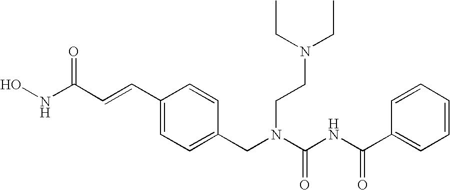 | 439 | 3-{4-[3-Benzoyl-1-(2-diethylamino-ethyl)-ureidomethyl]-phenyl}-N-hydroxy-acrylamide |
| L35 | 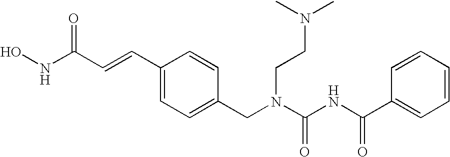 | 411 | 3-{4-[3-Benzoyl-1-(2-dimethylamino-ethyl)-ureidomethyl]-phenyl}-N-hydroxy-acrylamide |
| L36 | 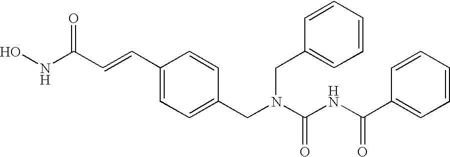 | 429 | 3-[4-(3-Benzoyl-1-benzyl-ureidomethyl)-phenyl]-N-hydroxy-acrylamide |

By methods analogous to those disclosed above and by varying the starting materials used in the synthesis, a wide variety of compounds of Formula (I) could be prepared, including, but not limited to, those in Table 2.

Non-commercial available acyl isocyanates could be prepared according to the published literature methods. For example, acetyl isocyanate could be synthesized by reacting Et₃SnNCO with acetyl bromide [Chauzov, V. A.; Baukov, Yu. I. Zhurnal Obshchei Khimii (1972), 42(8), 1868-9], or by reacting Bu₃SnNCO with acetyl chloride [Kodama, H. et al. Jpn. Tokkyo Koho (1972) JP47009568]. Acyl isocyanate R′CONCO [R′═C1-4 alkyl, (substituted) Ph, naphthyl] could be made by reaction of R′COX (X=halo) with NaOCN [Caubere, P. et al. Eur. Pat. Appl. (1989), EP 334720 A1]

TABLE 2

| | |
|---|---|
| V01 | 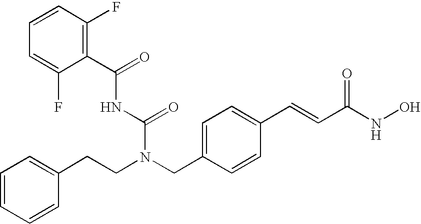 |
| V02 | 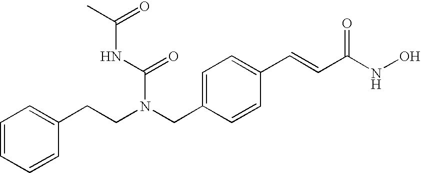 |
| V03 | 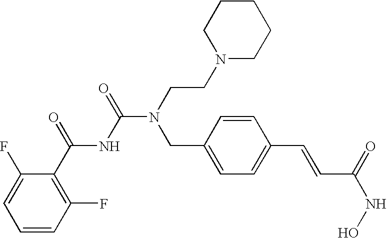 |
| V04 | 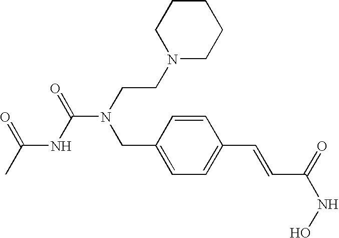 |
| V05 | 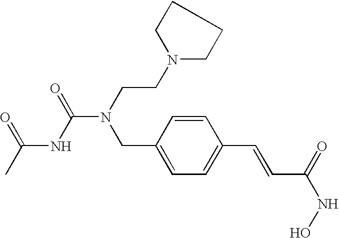 |
| V06 | 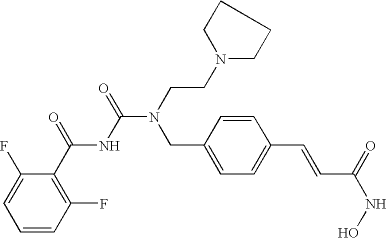 |
| V07 | 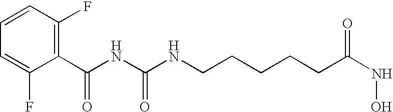 |
| V08 | 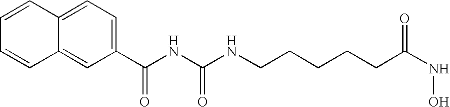 |
| V09 | 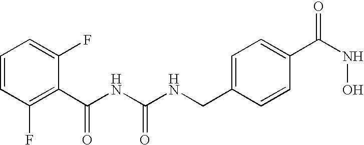 |
| V10 | 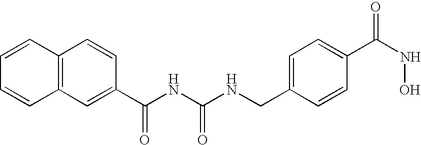 |
| V11 | 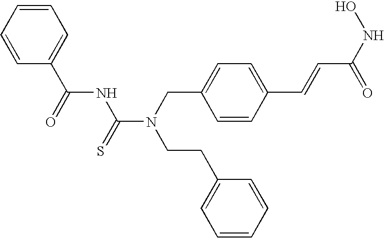 |
| V12 | 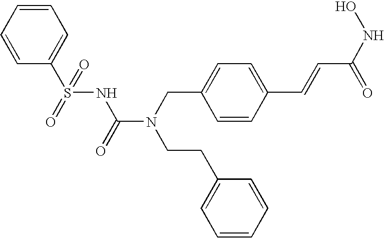 |
| V13 | 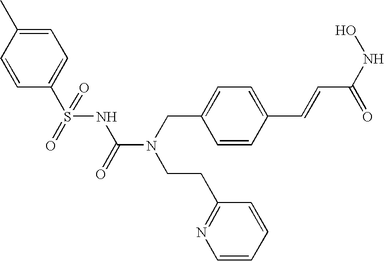 |
| V14 | 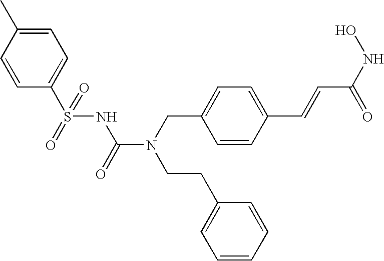 |
| V15 | 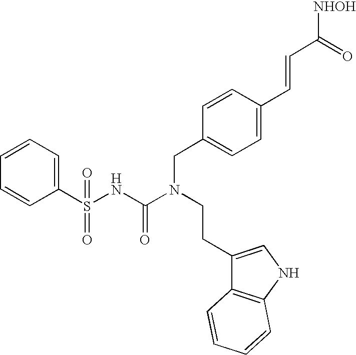 |
| V16 | 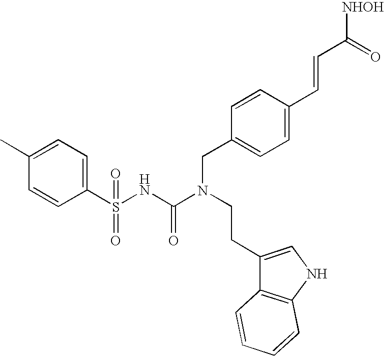 |
| V17 | 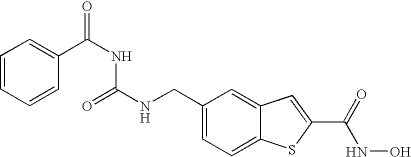 |
| V18 | 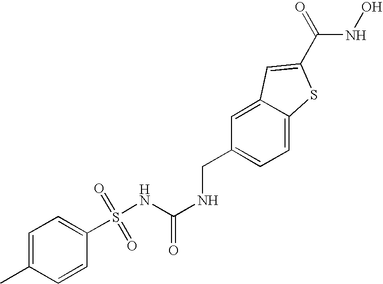 |
| V19 | 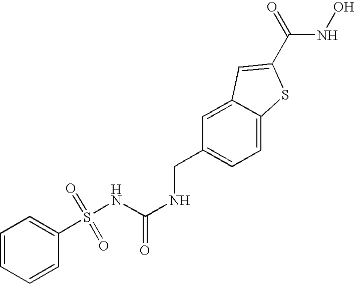 |
| V20 | 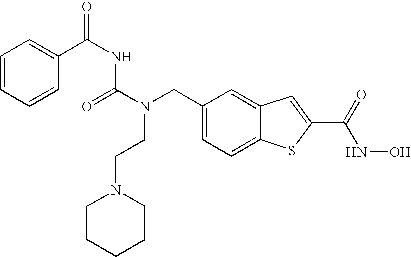 |
| V21 | 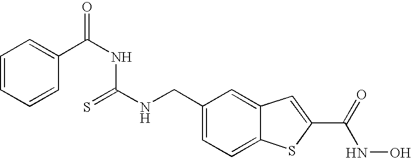 |
| V22 | 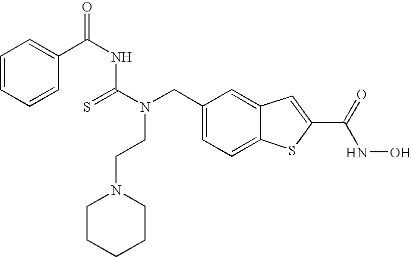 |
| V23 | 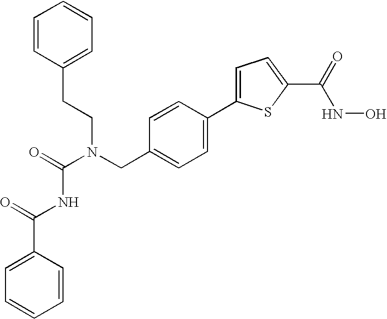 |
| V24 | 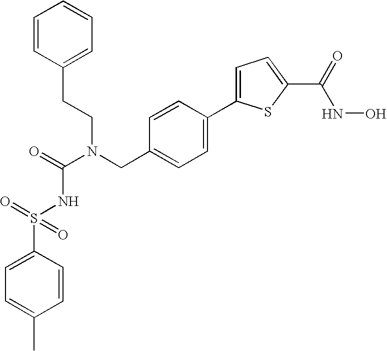 |
| V25 | 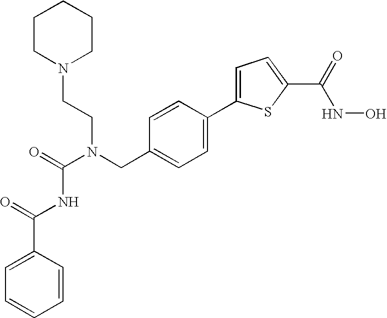 |
| V26 | 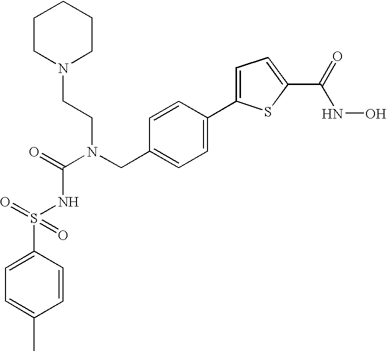 |
| V27 | 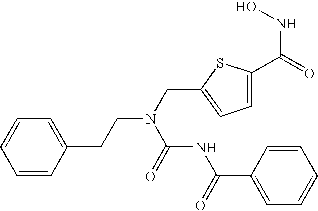 |
| V28 | 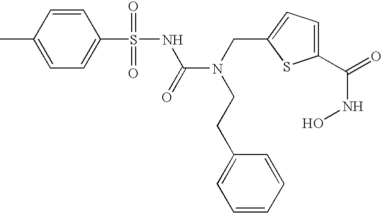 |
| V29 | 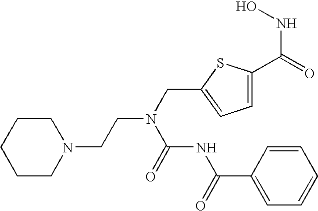 |
| V30 | 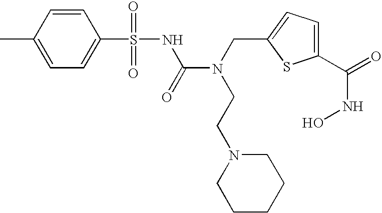 |
| V31 | 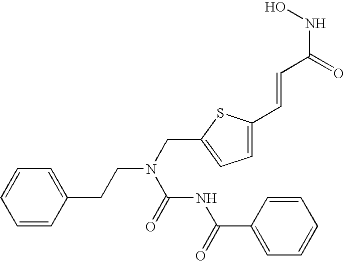 |
| V32 | 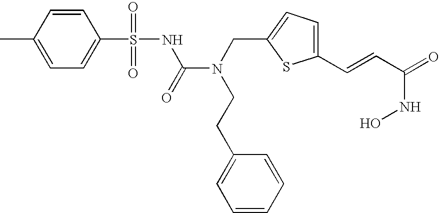 |
| V33 | 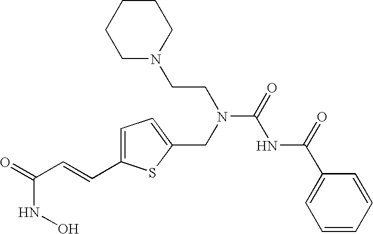 |
| V34 | 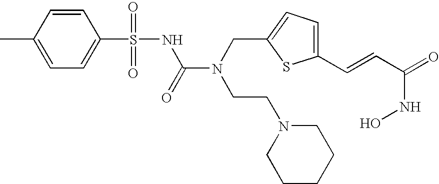 |
| V35 | 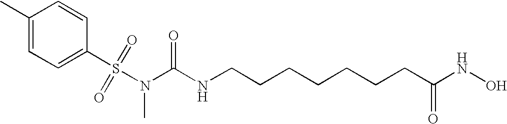 |
| V36 | 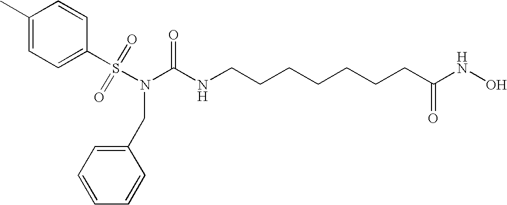 |
| V37 | 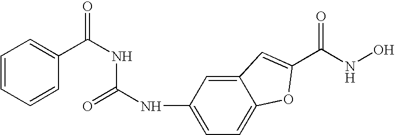 |
| V38 | 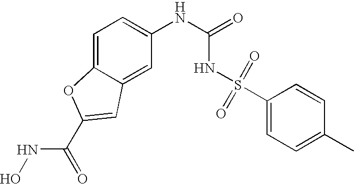 |
| V39 | 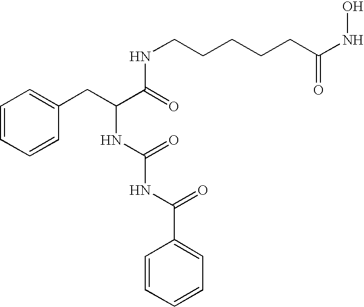 |
| V40 | 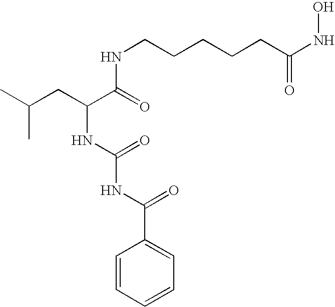 |
| V41 | 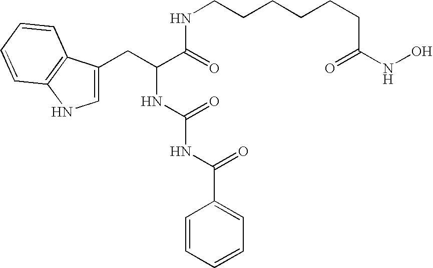 |
| V42 | 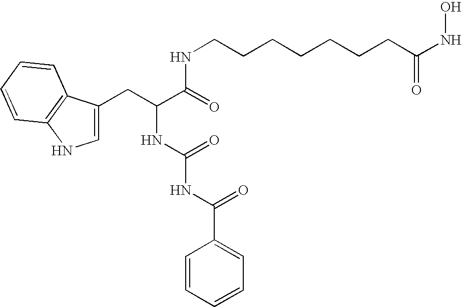 |
| V43 | 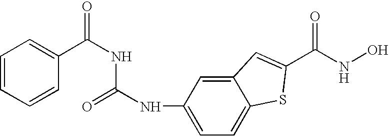 |
| V44 | 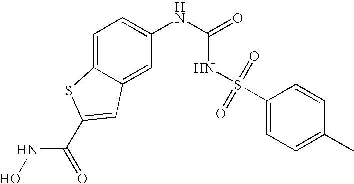 |

### Biological Testing and Enzyme Assays

### Recombinant GST-HDAC1 and GST-HDAC8 Protein Expression and Purification

Human cDNA library was prepared using cultured SW620 cells. Amplification of human HDAC1 and HDAC8 coding region from this cDNA library was cloned separately into the baculovirus expression pDEST20 vector and pFASTBAC vector respectively (GATEWAY Cloning Technology, Invitrogen Pte Ltd). The pDEST20-HDAC1 and pFASTBAC-HTGST-HDAC8 constructs were confirmed by DNA sequencing. Recombinant baculovirus was prepared using the Bac-To-Bac method following the manufacturer's instruction (Invitrogen Pte Ltd). Baculovirus titer was determined by plaque assay to be about 10⁸ PFU/ml.

Expression of GST-HDAC1 or HTGST-HDAC8 was done by infecting SF9 cells (Invitrogen Pte Ltd) with pDEST20-HDAC1 or pFASTBAC-GST-HDAC8 baculovirus at MOI=1 for 48 h. Soluble cell lysate was incubated with pre-equilibrated Glutathione Sepharose 4B beads (Amersham) at 4° C. for 2 h. The beads were washed with PBS buffer for 3 times. The GST-HDAC1 protein or GST-HDAC8 protein was eluted by elution buffer containing 50 mM Tris, pH8.0, 150 mM NaCl, 1% Triton X-100 and 10 mM or 20 mM reduced Glutathione. The purified GST-HDAC1 protein or purified GST-HDAC8 protein was dialyzed with HDAC storage buffer containing 10 mM Tris, pH7.5, 100 mM NaCl and 3 mM MgCl₂. 20% Glycerol was added to purified GST-HDAC1 protein or purified GST-HDAC8 before storage at −80° C.

### In Vitro HDAC Assay for Determination of IC₅₀ Values

The assay has been carried out in 96 well format and the BIOMOL fluorescent-based HDAC activity assay has been applied. The reaction composed of assay buffer, containing 25 mM Tris pH 7.5, 137 mM NaCl, 2.7 mM KCl, 1 mM MgCl₂, 1 mg/ml BSA, tested compounds, 500 nM HDAC8 enzyme or 600 nM HDAC1 enzyme, 200 μM Flur de lys p53 peptide substrate for HDAC8 enzyme or 500 μM Flur de lys generic substrate for HDAC1 enzyme and subsequently was incubated at room temperature for 2 h. Flur de lys Developer was added and the reaction was incubated for 10 min. Briefly, deacetylation of the substrate sensitizes it to the developer, which then generates a fluorophore. The fluorophore is excited with 360 nm light and the emitted light (460 nm) is detected on a fluorometric plate reader (Tecan Ultra Microplate detection system, Tecan Group Ltd.).

### The analytical software, Prism 3.0® (GraphPad Software Inc) has been used to generate IC₅₀ from a series of data.

The HDAC enzyme inhibition results of representative compounds are shown in Table 3.

TABLE 3
HDAC enzyme inhibition activities of representative examples

| Compound | HDAC1 IC₅₀ (μM) | HDAC8 IC₅₀ (μM) |
|---|---|---|
| Example 1 | >100 | 0.79 |
| Example 2 | 2.61 | 0.040 |
| Example 3 | 1.54 | 0.022 |
| Example 4 | 2.92 | 0.049 |
| Example 5 | >100 | 0.14 |
| Example 6 | 0.13 | 0.041 |
| Example 7 | >100 | 0.15 |
| Example 8 | >100 | 0.072 |
| Example 10 | 0.056 | 1.07 |
| Example 11 | 0.004 | 0.21 |
| Example 12 | 0.098 | 0.40 |
| Example 13 | 0.15 | 0.27 |
| Example 14 | 1.13 | 0.051 |
| Example 18 | 0.027 | 0.52 |
| Example 23 | 0.012 | 0.20 |
| Example 30 | 2.87 | 0.46 |
| Example 46 | 0.048 | 0.23 |
| Example 47 | 0.024 | 0.25 |

### Cell-Based Proliferation Assay for Determination of GI₅₀ Values

Human colon cancer cell lines (Colo205) and human breast cancer cell lines (MDA-MB435 and MDA-MB231) were obtained from ATCC. Colo205 cells were cultivated in RPMI 1640 containing 2 mM L-Glutamine, 5% FBS, 1.0 mM Na Pyruvate. MDA-MB231 cells were cultivated in RPMI 1640 containing 2 mM L-glutamine, 5% FBS. MDA-MB435 cells were cultivated in DMEM containing 2 mM L-Glutamine, 5% FBS. Colo205 cells were seeded in 96-wells plate at 5000 cells per well respectively. MDA-MB435 and MDA-MB231 cells were seeded in 96-wells plate at 6000 cells per well. The plates were incubated at 37° C., 5% CO₂, for 24 h. Cells were treated with compounds at various concentrations for 96 h. Cell growth was then monitored using cyquant cell proliferation assay (Invitrogen Pte Ltd). Dose response curves were plotted to determine GI₅₀ values for the compounds using XL-fit (ID Business Solution, Emeryville, Calif.).

The cellular or growth inhibition activity results of representative compounds are shown in Table 4. These data indicate that compounds in this invention are highly active in inhibition of tumor cell growth. In addition, representative compounds have also demonstrated their ability to inhibit growth in other types of cancer cell lines including lung cancer cell lines (e.g. NCI-H522 and A549), prostate cancer cell line (e.g. PC3), leukemia cell line (e.g. HL-60), lymphoma cell line (e.g. Ramos) and pancreatic cancer cell line (MIAPaCA2) (data not shown).

TABLE 4
Cellular activities of representative examples

| | Colo 205 | MDA-MB231 | MDA-MB435 |
|---|---|---|---|
| Compound | Gl₅₀ (μM) | Gl₅₀ (μM) | Gl₅₀ (μM) |
| Example 6 | 1.91 | 1.92 | 1.24 |
| Example 10 | 1.89 | 1.03 | 1.79 |
| Example 11 | 0.26 | 0.26 | 0.62 |
| Example 12 | 8.03 | | |
| Example 13 | 2.68 | | |
| Example 18 | 2.67 | | |
| Example 23 | 0.15 | 0.26 | |
| Example 25 | 0.16 | | |
| Example 46 | 0.63 | | |

### Histone H3, H4. H2A and H₂B Acetylation Assay

A hallmark of histone deacetylase (HDAC) inhibition is the increase in the acetylation level of histones. Histone acetylation, including H3, H4, H2A and H₂B can be detected by immuno-blotting (western-blot). Colo205 cells, approximately 1.5×10⁶ cells/10 cm dish, were seeded in the previously described medium, cultivated for 24 h and subsequently treated with HDAC inhibitory agents at 0.1, 1, 5 and 10 μM final concentration. After 24 h, cells were harvested and lysed according to the instruction from Sigma Mammalian Cell Lysis Kit. The protein concentration was quantified using BCA method (Sigma Pte Ltd). The protein lysate was separated using 4-12% bis-tris SDS-PAGE gel (Invitrogen Pte Ltd) and was transferred onto PVDF membrane (BioRad Pte Ltd). The membrane was probed separately using primary antibody specific for acetylated H3, acetylated H4 or acetylated H2A (Upstate Pte Ltd). The detection antibody, goat anti rabbit antibody conjugated with Horse radish peroxidase (HRP) was used according to the manufacturer instruction (Pierce Pte Ltd). After removing the detection antibody from the membrane, an enhanced chemiluminescent substrate for detection of HRP (Pierce Pte Ltd) was added onto the membrane. After removing the substrate, the membrane was exposed to an X-ray film (Kodak) for 1 sec-20 mins. The X-ray film was developed using the X-ray film processor. The density of each band observed on the developed film could be analysed using UVP Bioimaging software (UVP, Inc, Upland, Calif.). The values were then normalized against the density of actin in the corresponding samples to obtain the expression of the protein. The results of histone deacetylase assay are shown in Table 5.

TABLE 5
Effects of representative examples on accumulation of acetylated histone.

| | Histone 3 | Histone 4 | Hisotne 2A | Histone 2B |
|---|---|---|---|---|
| Compound | acetylation | acetylation | acetylation | acetylation |
| Example 6 | Active | Active | | |
| Example 10 | Active | Active | Active | Active |
| Example 11 | Active | Active | Active | Active |
| Example 13 | Active | | | |
| Example 18 | Active | | | |
| Example 23 | Active | | | |
| Example 46 | Active | | | |

“Active” means accumulation of acetylated histone was observed when compared with control (without compound).

These data demonstrate that compounds in this invention inhibit histone deacetylases, thereby resulting in accumulation of acetylated histones.

### Apoptosis Assays

In various therapies such as for proliferative disorders like cancer, the selective induction of apoptosis in proliferating cells such as tumor cells is one of the desirable approaches, and can be mediated by treatment with various anti-proliferative compounds [Blagosklonny M V, Oncogene, 23(16): 2967 (2004); Kaufmann and Earnshaw, Exp Cell Res. 256(1): 42-9 (2000)]. Programmed cell death or apoptosis is the cellular response to stress factors such as DNA damage introduced during conventional anti-cancer treatment. The concerted sequence of events during apoptosis, clearly differentiate this pathway from a non-coordinated form of cell death called necrosis. During the course of apoptosis, characteristic phenotypical cellular changes occur, which include the condensation of chromatin, the shrinkage of cells and finally the fragmentation of chromosomal DNA. One of the very early changes caused by apoptotic events occurs in the phospholipids bilayer of the plasma membrane. The phospholipid phosphatidylserine is translocated from the inner to the outer side of the plasma-membrane and, as a result, is exposed to the extracellular space. One way of detecting early apoptotic cells is to determine the amount of phosphatidyl-serine at the extracellular side of the plasma-membrane which is accomplished by the standard flow cytometric method of Annexin V staining. The phospholipids recognizing protein Annexin V binds with high affinity to these inverted and exposed phosphatidyl-serines.

The ability of the compounds in this invention to induce apoptosis was tested in Ramos Burkitt-lymphoma cells. This cell line is one of the gold standard cell lines commonly used as a tissue culture model for B cell lymphoma. Representative compounds as indicated below were added to 80,000 cells per 500 μl growth medium (RPMI1640 medium supplemented with 2 mM L-Glutamine, 10% heat-inactivated FBS, 1 mM Na-Pyruvate and 10 mM HEPES) in 24 well format at various concentrations. Two days after the start of treatment, cells were collected and subjected to the Annexin V staining protocol following the instructions of the manufacturer (BD Biosciences). By using propidium iodide (PI) as a viability control, cells that stain positive for Annexin V, but negative for PI, are undergoing apoptosis. The percentage of cells in late apoptosis after treatment was derived from a standard flow cytometry (FACS) analysis [Steensma et al, Methods Mol Med 85:323-32 (2003). For example, the percentage of late apoptotic cells 48 hr after treatment with 10 μM was 84% for compound Example 3 (N-Hydroxy-3-{3-[3-(4-methylbenzenesulfonyl)ureido]-phenyl}acrylamide). In addition, selected compounds are tested for their ability to induce apoptosis in HL-60 cells which is an acute promyelocytic leukemia cell line (data not shown). Hence, compounds disclosed in this invention can be used to treat cancers including hematologic malignancies (e.g. lymphoma and leukemia).

### In Vivo Xenograft Tumor Study

In data not shown, selected compounds were tested for maximal tolerated dose in normal mice and were found to be well tolerated by the mice with no obvious signs of toxicity or side effects in the dose range applied (which can be >200 mg/kg/day).

The efficacy of the compounds of the invention can then be determined using in vivo animal xenograft studies. The animal xenograft model is one of the most commonly used in vivo cancer models.

In these studies Female athymic nude mice (Harlan), 12-14 weeks of age would be implanted subcutaneously in the flank with 5×10⁶ cells of HCT116 or with 1×10⁶ cells of Colo205 human colon carcinoma suspended in 50% Matrigel. When the tumor reaches the size 100 mm³, the xenograft nude mice would be paired-match into various treatment groups. The selected HDAC inhibitors would be dissolved in appropriate vehicles, such as 10% DMA/10% Cremophore/80% water and administered to xenograft nude mice intraperitonelly by daily for 14 days. The dosing volume will be 0.2-ml/20 g mouse. Paclitaxol, used as positive control, will be prepared for intravenous administration in 10% Ethanol/10% Cremophore/80% water. The dosing volume for Paclitaxol will be 0.015-ml/g mouse. Tumor volume will be calculated every second day of post injection using the formula: Tumor volume (mm³)=(w²×l)/2, where w=width and l=length in mm of an HCT116 or Colo205 carcinoma [Beverly A T, In Tumor Models in Cancer Research, published by Humana Press, New Jersey, p 593-612, 2002]. Compounds in this invention that are tested would show significant reduction in tumor volume relative to controls treated with vehicle only. The activity of histone deacetylase when measured shall be reduced and results in accumulation of acetylated histone relative to vehicle treated control group. The result will therefore indicate that compounds in this invention are efficacious in treating a proliferative disorder such as cancer.

The details of specific embodiments described in this invention are not to be construed as limitations. Various equivalents and modifications may be made without departing from the essence and scope of this invention, and it is understood that such equivalent embodiments are part of this invention.

1-79. (canceled)

80. A compound of the Formula (I) 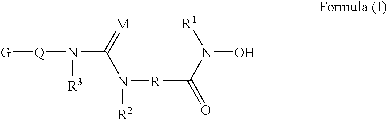
wherein
R is a linking moiety;
R¹ is selected from the group consisting of H, C₁-C₆ alkyl and acyl;
M is selected from the group consisting of O, S, NH, NR⁴, NOH and NOR⁴;
R² is selected from the group consisting of H, halogen, alkyl, alkenyl, alkynyl, haloalkyl, haloalkenyl, heteroalkyl, cycloalkyl, cycloalkenyl, heterocycloalkyl, heterocyclocloakenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴, NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted,
or
R² together with the nitrogen to which it is attached and a portion of R form an optionally substituted heterocycloalkyl group;
R³ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴¹ NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted;
Q is selected from the group consisting of —S(O)₂—, —C(═O)— and —C(═S);
G is selected from the group consisting of optionally substituted alkyl, optionally substituted cycloalkyl, optionally substituted aryl, optionally substituted heteroaryl, optionally substituted heterocycloalkyl, optionally substituted arylalkyl, and optionally substituted heteroarylalkyl;
each R⁴ is independently selected from the group consisting of H, alkyl, alkenyl, alkynyl, haloalkyl, heteroalkyl, cycloalkyl, heterocycloalkyl, aryl, heteroaryl, cycloalylalkyl, heterocycloalkylalkyl, arylalkyl, heteroarylalkyl and acyl, each of which may be optionally substituted;
or a pharmaceutically acceptable salt or prodrug thereof, wherein when R is methyl or isopropylmethyl then R² is not benzyl.

81. A compound according to claim 80 having the Formula (2) 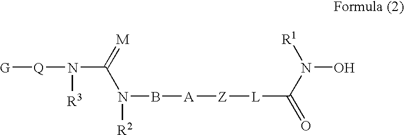
wherein
R¹ is selected from the group consisting of H, C₁-C₆ alkyl and acyl;
L is a single bond or is a C₁-C₅ hydrocarbon chain which may contain 0 to 2 multiple bonds independently selected from double bonds and triple bonds and wherein, the chain may optionally be interrupted by at least one of —O—, —S—, —S(O)— and —S(O)₂— and the chain may optionally be substituted with one or more substituents independently selected from the group consisting of C₁-C₄ alkyl;
Z is selected from the group consisting of a single bond, N(R¹), O, S, S(O) and S(O)₂;
A is selected from the group consisting of a single bond, optionally substituted arylene, optionally substituted heteroarylene, optionally substituted cycloalkylene and optionally substituted heterocycloalkylene;
B is selected from the group consisting of a single bond, optionally substituted aminoacyl, optionally substituted arylene, optionally substituted heteroarylene, optionally substituted arylalkylene, optionally substituted heteroarylalkylene, optionally substituted alkylarylene, optionally substituted alkylheteroarylene, optionally substituted C₁-C₃ alkylene, optionally substituted heteroalkylene, optionally substituted cycloalkylene, optionally substituted heterocycloalkylene and optionally substituted —(CH₂)ₘ—C(O)—N(R⁴)—(CH₂)ₙ—, wherein n is an integer from 0 to 6, m is an integer from 0 to 6;
M is selected from the group consisting of O, S, NH, NR⁴, NOH and NOR⁴;
R² is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR₄, NHCOR⁴, NHCOOR⁴NHCONHR⁴, C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted,
or
R² together with the nitrogen to which it is attached and a portion of B form an optionally substituted heterocycloalkyl group;
R³ is independently selected from the group consisting of H, halogen alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, aryalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, sulfonylamino, sulfinylamino, phenoxy, benzyloxy, COOR⁴, CONHR⁴, NHCOR⁴, NHCOOR⁴, NHCONHR⁴C(═NOH)R⁴, alkoxycarbonyl, alkylaminocarbonyl, sulfonyl, alkylsulfonyl, alkylsulfinyl, arylsulfonyl, arylsulfinyl, aminosulfonyl, aminosulfinyl, SR⁴ and acyl; each of which may optionally be substituted;
Q is selected from the group consisting of —S(O)₂—, —C(═O)— and C(═S)—;
G is selected from the group consisting of optionally substituted aryl, optionally substituted heteroaryl, optionally substituted alkyl, optionally substituted cycloalkyl, optionally substituted heterocycloalkyl, optionally substituted arylalkyl and optionally substituted heteroarylalkyl;
each R⁴ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl; each of which may be optionally substituted;
or a pharmaceutically acceptable salt or prodrug thereof.

82. A compound according to claim 81 having the Formula (2a) 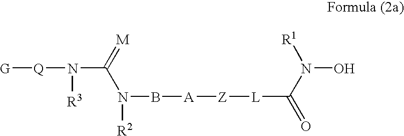
wherein
R¹ is selected from the group consisting of H, C₁-C₆ alkyl and acyl;
L is a single bond or is a C₁-C₅ hydrocarbon chain which may contain 0 to 2 multiple bonds independently selected from double bonds and triple bonds and wherein, the chain may optionally be interrupted by at least one of —O—, —S—, —S(O)— and —S(O)₂— and the chain may optionally be substituted with one or more substituents independently selected from the group consisting of C₁-C₄ alkyl;
Z is selected from the group consisting of a single bond, N(R¹) O, S, S(O) and S(O)₂;
A is selected from the group consisting of a single bond, optionally substituted arylene, optionally substituted heteroarylene, optionally substituted cycloalkylene and optionally substituted heterocycloalkylene;
B is selected from the group consisting of a single bond, optionally substituted aminoacyl, optionally substituted arylene, optionally substituted heteroarylene, optionally substituted arylalkylene, optionally substituted heteroarylalkylene, optionally substituted alkylarylene, optionally substituted alkylheteroarylene, optionally substituted C₁-C₃ alkylene, optionally substituted heteroalkylene, optionally substituted cycloalkylene optionally substituted heterocycloalkylene and optionally substituted —(CH₂)ₘ—C(O)—N(R⁴)—(CH₂)ₙ—, wherein n is an integer from 0 to 6, m is an integer from 0 to 6;
M is selected from the group consisting of O, S, NH, NR⁴, NOH and NOR⁴;
R² is selected from the group consisting of H, C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylosulfonyl, arylsulfonyl, aminosulfonyl, —C(O)OR⁴, —CONHR⁴, —NHCONHR⁴, C(═NOH)R⁴, and acyl;
R³ is selected from the group consisting of H, C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylosulfonyl, arylsulfonyl, aminosulfonyl, C(O)OR⁴, —CONR⁴, —NHCONHR⁴, C(═NOH)R⁴, and acyl;
Q is selected from the group consisting of —S(O)₂—, —CO— and —C(═S %;
G is selected from optionally substituted aryl, optionally substituted heteroaryl, alkyl, optionally substituted cycloalkyl, optionally substituted heterocycloalkyl, optionally substituted arylalkyl and optionally substituted heteroarylalkyl, wherein the substituents are independently selected from the group consisting of X, Y, R⁴, hydroxyl, hydroxyalkyl, alkoxy, amino, alkylamino, aminoalkyl, acylamino, phenoxy, alkoxyalkyl, benzyloxy, alkylosulfonyl, arylsulfonyl, aminosulfonyl, —C(O)OR⁴, —C(O)OH, —SH, —CONHR⁴, —NHCONHR⁴, and C(—NOH)R⁴;
R⁴ is selected from the group consisting of C₁-C₄ alkyl, heteroalkyl, aryl, heteroaryl and acyl;
X and Y are the same or different and are independently selected from the group consisting of H, halo, C₁-C₄ alkyl, NO₂, OR⁴, SR⁴, C(O)R⁵, and NR⁶R⁷;
R⁵ is C₁-C₄ alkyl;
R⁶ and R⁷ are the same or different and are independently selected from the group consisting of H, C₁-C₆ alkyl, C₄-C₉ cycloalkyl, C₄-C₉ heterocycloalkyl, aryl, heteroaryl, arylalkyl and heteroaryl alkyl.
or a pharmaceutically acceptable salt or prodrug thereof.

83. A compound according to claim 81 having the Formula (2b) 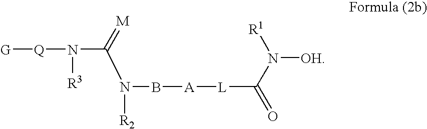
or a pharmaceutically acceptable salt or prodrug thereof.

84. A compound according to claim 81 having the Formula (2c) 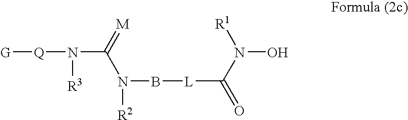
or a pharmaceutically acceptable salt or prodrug thereof.

85. A compound according to claim 81 wherein A is optionally substituted arylene.

86. A compound according to claim 81 wherein A selected from the group consisting of 1,4-phenylene and 1,3-phenylene.

87. A compound according to claim 81 wherein A is 1,4-phenylene.

88. A compound according to claim 81 wherein L is selected from the group consisting of a single bond, —CH₂—, —(CH₂)₂— and —CH═CH—.

89. A compound according to claim 81 wherein L is a bond.

90. A compound according to claim 81 wherein L is a group of formula —CH₂—.

91. A compound according to claim 81 wherein L is a group of formula —CH═CH—.

92. A compound according to claim 81 wherein B is selected from the group consisting of a single bond, methylene, ethylene, propylene, alkylarylene, and heteroalkylene.

93. A compound according to claim 81 wherein B is methylene.

94. A compound according to claim 81 wherein B is a single bond.

95. A compound according to claim 81 wherein B is ethylene.

96. A compound according to claim 81 wherein B is propylene.

97. A compound according to claim 81 wherein the group BAZL is a group of formula —(CH₂)ₙ— wherein n is an integer from 1 to 7.

98. A compound according to claim 81 wherein the group BAZ is a group of formula —(CH₂)— phenyl-.

99. A compound according to claim 81 wherein the group BAZL is selected from the group consisting of 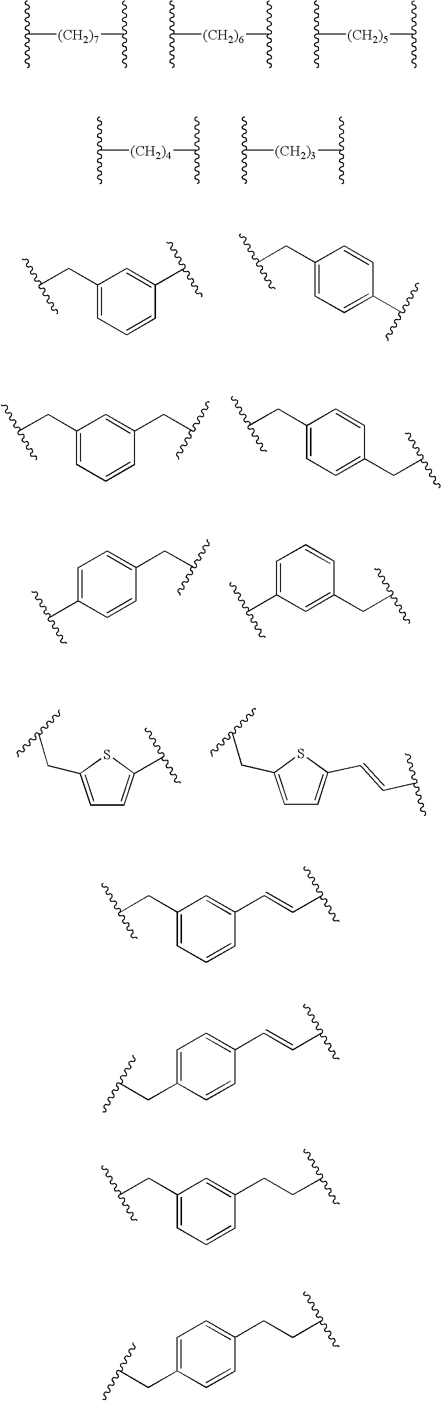 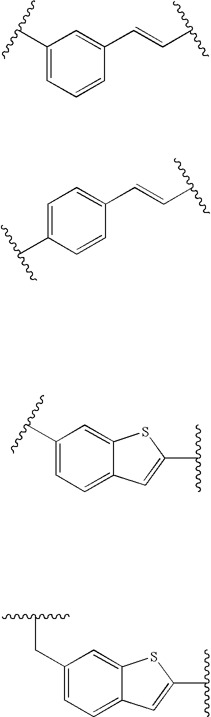 [symbol]
is a single bond

100. A compound according to claim 81 wherein R² and a portion of B together with the nitrogen to which they are attached form a heterocycloalkylene.

101. A compound according to claim 100 wherein the heterocycloalkylene is 1,4-piperazinylene.

102. A compound according to claim 81 wherein R¹═H.

103. A compound according to claim 81 wherein M is O.

104. A compound according to claim 81 wherein M is S.

105. A compound according to claim 81 wherein Q is S(O)₂.

106. A compound according to claim 81 wherein Q is CO.

107. A compound according to claim 81 wherein G is optionally substituted aryl.

108. A compound according to claim 81 wherein G is phenyl.

109. A compound according to claim 81 wherein G is 4-methylphenyl.

110. A compound according to claim 81 wherein R² is selected from the group consisting of H, optionally substituted alkyl, optionally substituted heteroalkyl, optionally substituted cycloalkyl, optionally substituted heterocycloalkyl, optionally substituted aryl, optionally substituted heteroaryl, optionally substituted arylalkyl, optionally substituted arylheteroalkyl, optionally substituted heteroarylalkyl, optionally substituted heteroarylheteroalkyl, optionally substituted cycloalkylalkyl and optionally substituted heterocycloalkylalkyl.

111. A compound according to claim 81 wherein R² is selected from the group consisting of H, 2-(1H-indol-3-yl)-ethyl, 2-(2-methyl-1H-indol-3-yl)-ethyl, pyridin-3-ylmethyl, 3-hydroxy-propyl, 2-pyridin-2-yl-ethyl, 2-pyridin-3-yl-ethyl, pyridin-3-ylmethyl, 2-pyridin-4-yl-ethyl, benzyl, 3-phenyl-propyl, 2-phenoxy-ethyl, morpholin-4-yl, pyridin-2-yl, phenethyl, 2-(4-bromo-phenyl)-ethyl, 2-(4-fluoro-phenyl)-ethyl, 3-imidazol-1-yl-propyl, 2-(1H-imidazol-4-yl)-ethyl, 1H-Benzoimidazol-2-ylmethyl, 2-piperidin-1-yl-ethyl, 2-pyrrolidin-1-yl-ethyl, 2-cyclohex-1-enyl-ethyl, 2-ethyl-hexyl, 2-thiophen-2-yl-ethyl, 3,3-diphenyl-propyl, 2-biphenyl-4-yl-ethyl, -(4-phenoxy-phenyl, 2-(3-phenoxy-phenyl)-ethyl, 2-(2,3-dimethoxy-phenyl, 2-(2,4-dichloro-phenyl)-ethyl, cyclohexylmethyl, hexyl, isobutyl, 3-isopropoxy-propyl, 2-phenoxy-ethyl, 2-isopropoxy-ethyl, 3-methoxy-benzyl, 4-[1,2,3]thiadiazol-4-yl-benzyl, 2,4-dichloro-benzyl, 2-(2-methoxy-phenyl)-ethyl, 2-(3-fluoro-phenyl)-ethyl, 2-(2-fluoro-phenyl)-ethyl, 2,2-diphenyl-ethyl, 2-(4-methoxy-phenyl)-ethyl, 2-(3-chloro-phenyl)-ethyl, 4-phenyl-butyl, 3-phenyl-propyl, 3,3-diphenyl-propyl, 3-(4-methyl-piperazin-1-yl, 3-morpholin-4-yl-propyl, 3-(2-oxo-pyrrolidin-1-yl)-propyl, 3-pyrrolidin-1-yl-propyl, tetrahydro-furan-2-ylmethyl, 1,5-dimethyl-hexyl, 2-diethylaminoethyl and 2-dimethylamino-ethyl.

112. A compound according to claim 81 wherein R² is selected from the group consisting of H, 2-(1H-indol-3-yl)-ethyl, 2-(2-methyl-1H-indol-3-yl)-ethyl, pyridin-3-ylmethyl, 3-hydroxy-propyl, 2-pyridin-2-yl-ethyl, 2-pyridin-3-yl-ethyl, pyridin-2-ylmethyl, pyridin-3-ylmethyl, 2-pyridin-4-yl-ethyl, benzyl, 3-phenyl-propyl, 2-phenoxy-ethyl, 2-morpholino ethyl, 2-phenyl ethyl, 2-(4-bromo-phenyl)-ethyl, 2-(4-fluoro-phenyl)-ethyl, 3-imidazol-1-yl-propyl, 2-(1H-imidazol-4-yl)-ethyl, 1H-Benzoimidazol-2-ylmethyl, 2-piperidin-1-yl-ethyl and 2-pyrrolidin-1-yl-ethyl.

113. A compound according to claim 81 wherein R¹ is selected from the group consisting of H, 2-(1H-indol-3-yl)-ethyl, 2-(2-methyl-1H-indol-3-yl)-ethyl, 2-phenyl ethyl, 2-piperidin-1-yl-ethyl and 2-pyrrolidin-1-yl-ethyl.

114. A compound according to claim 81 wherein the optional substituents are selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkoxy, alkoxyalkyl, alkoxycycloalkyl, alkoxyheterocycloalkyl, alkoxyaryl, alkoxyheteroaryl, alkoxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, arylalkoxy, heteroarylalkyl, cycloalkylalkyl, heterocycloalkylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —COOH, —COR⁵, —C(O)OR⁵, CONHR⁵, NHCOR⁵, NHCOOR⁵, NHCONHR⁵, C(═NOH)R⁵, —SH, —SR⁵, —OR⁵ and acyl,
wherein each R⁵ is independently selected from the group consisting of alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted.

115. A compound according to claim 80 selected from the group consisting of

| | |
|---|---|
| 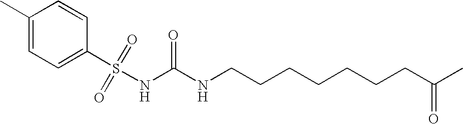 | 8-[3-(4-methylbenzenesulfonyl)-ureido])-octanoic acid hydroxyamide, |
| 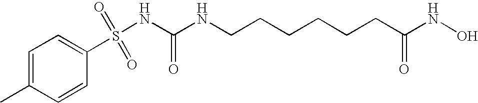 | 7-[3-(4-methylbenzenesulfonyl)-ureido])-heptanoic acidhydroxyamide, |
| 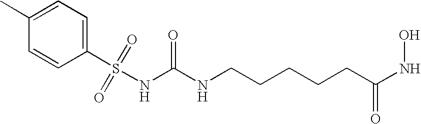 | 6-[3-(4-methylbenzenesulfonyl)-ureido])-hexanoic acidhydroxyamide, |
| 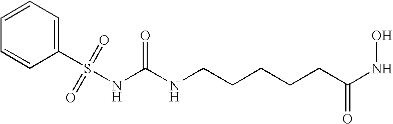 | 6-[3-(benzenesulfonyl)-ureido])-hexanoic acid hydroxyamide, |
| 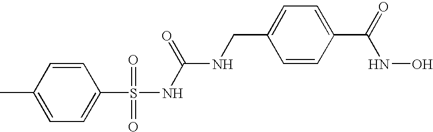 | N-Hydroxy-4-[3-(4-methylbenzenesulfonyl)ureido]methyl-benzamide, |
| 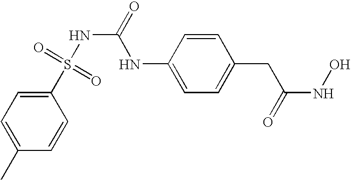 | N-Hydroxy-2-{4-[3-(4-methylbenzenesulfonyl)ureido]-phenyl}-acetamide, |
| 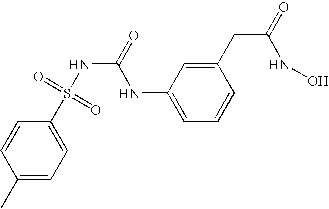 | N-Hydroxy-2-{3-[3-(4-methylbenzenesulfonyl)ureido]-phenyl}-acetamide, |
| 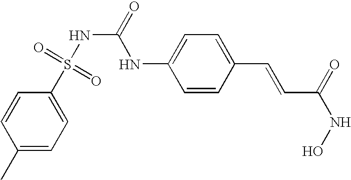 | N-Hydroxy-3-{4-[3-(4-methylbenzenesulfonyl)ureido]-phenyl}-acrylamide, |
| 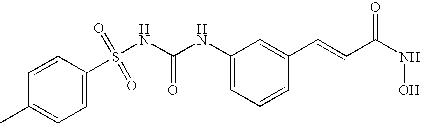 | N-Hydroxy-3-{3-[3-(4-methylbenzenesulfonyl)ureido]-phenyl}-acrylamide, |
| 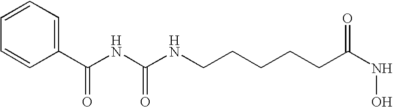 | 6-(3-Benzoyl-ureido)-hexanoic acidhydroxyamide, |
| 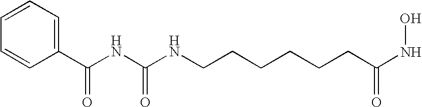 | 7-(3-Benzoyl-ureido)-heptanoic acidhydroxyamide, |
| 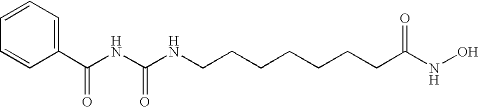 | 8-(3-Benzoyl-ureido)-octanoic acidhydroxyamide, |
| 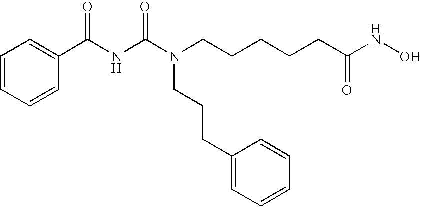 | 6-[3-Benzoyl-1-(3-phenyl-propyl)-ureido]-hexanoic acid hydroxyamide, |
| 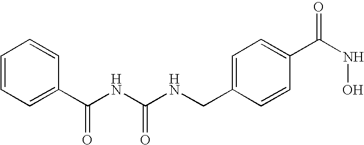 | 4-(3-Benzoyl-ureidomethyl)-N-hydroxy-benzamide, |
| 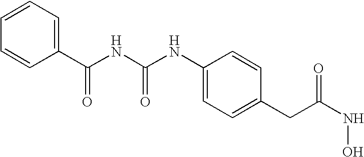 | 2-[4-(3-Benzoyl-ureido)-phenyl]-N-hydroxy-acetamide, |
| 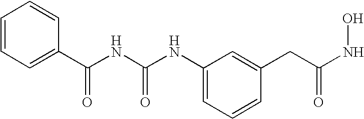 | 2-[3-(3-Benzoyl-ureido)-phenyl]-N-hydroxy-acetamide, |
| 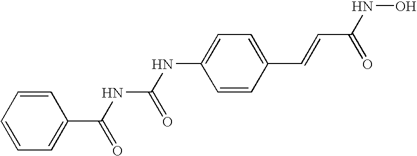 | 3-[4-(3-Benzoyl-ureido)-phenyl]-N-hydroxy-acrylamide, |
| 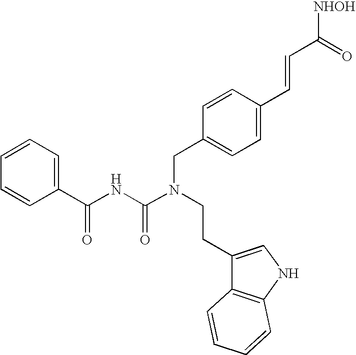 | 3-(4-{3-Benzoyl-1-[2-(1H-indol-3-yl)-ethyl]-ureidomethyl}-phenyl)-N-hydroxy-acrylamide, |
| 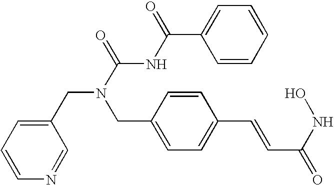 | 3-[4-(3-Benzoyl-1-pyridin-3-ylmethyl-ureidomethyl)-phenyl]-N-hydroxy-acrylamide, |
| 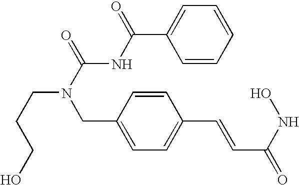 | 3-{4-[3-Benzoyl-1-(3-hydroxy-propyl)-ureidomethyl]-phenyl}-N-hydroxy-acrylamide, |
| 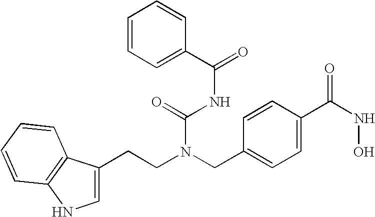 | 4-{3-Benzoyl-1-[2-(1H-indol-3-yl)-ethyl]-ureidomethyl}-N-hydroxy-benzamide, |
| 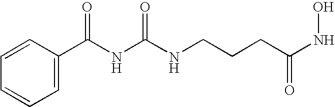 | 4-(3-Benzoyl-ureido)-N-hydroxy-butyramide, |
| 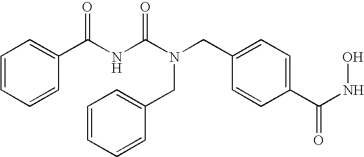 | 4-(3-Benzoyl-1-benzyl-ureidomethyl)-N-hydroxy-benzamide, |
| 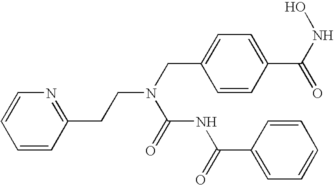 | 4-[3-Benzoyl-1-(2-pyridin-2-yl-ethyl)-ureidomethyl]-N-hydroxy-benzamide, |
| 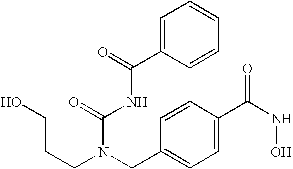 | 4-[3-Benzoyl-1-(3-hydroxy-propyl)-ureidomethyl]-N-hydroxy-benzamide, |
| 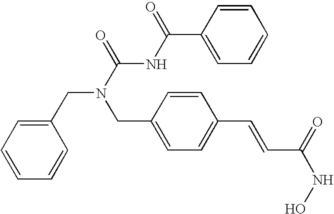 | 3-[4-(3-Benzoyl-1-benzyl-ureidomethyl)-phenyl]-N-hydroxy-acrylamide, |
| 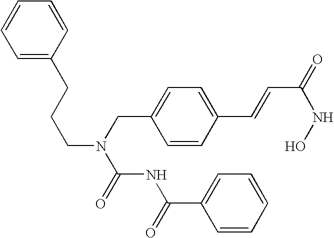 | 3-{4-[3-Benzoyl-1-(3-phenyl-propyl)-ureidomethyl]-phenyl}-N-hydroxy-acrylamide, |
| 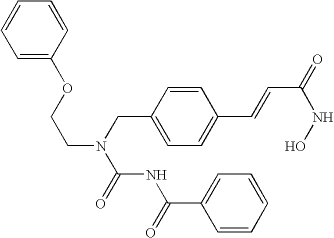 | 3-{4-[3-Benzoyl-1-(2-phenoxy-ethyl)-ureidomethyl]-phenyl}-N-hydroxy-acrylamide, |
| 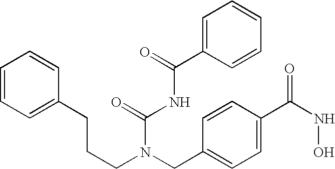 | 4-[3-Benzoyl-1-(3-phenyl-propyl)-ureidomethyl]-N-hydroxy-benzamide, |
| 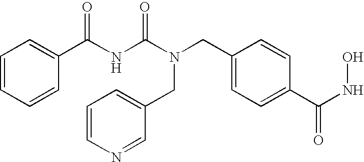 | 4-(3-Benzoyl-1-pyridin-3-ylmethyl-ureidomethyl)-N-hydroxy-benzamide, |
| 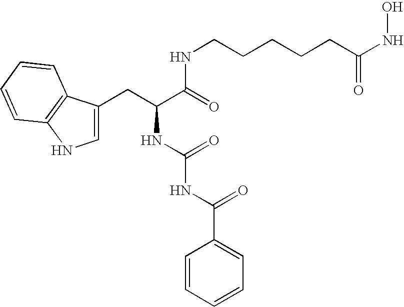 | (S)-6-[2-(3-Benzoyl-ureido)-3-(1H-indol-3-yl)-propionylamino]-hexanoic acid hydroxyamide, |
| 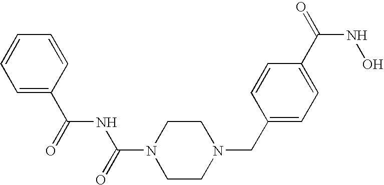 | 4-(4-Benzoylaminocarbonyl-piperazin-1-ylmethyl)-N-hydroxy-benzamide, |
| 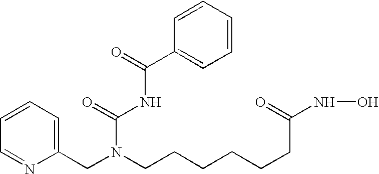 | 7-{(3-Benzoyl-1-pyridin-2-ylmethyl-ureido)-heptanoic acid hydroxyamide, |
| 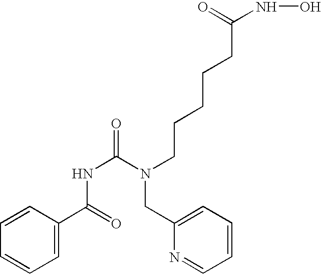 | 6-(3-Benzoyl-1-pyridin-2-ylmethyl-ureido)-hexanoic acid hydroxyamide, |
| 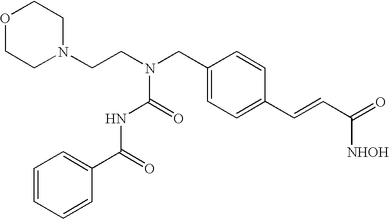 | 3-{4-[3-Benzoyl-1-(2-morpholin-4-yl-ethyl)-ureidomethyl]-phenyl}-N-hydroxy-acrylamide, |
| 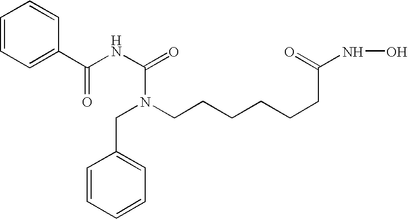 | 7-(3-Benzoyl-1-benzyl-ureido)-heptanoic acid hydroxyamide, |
| 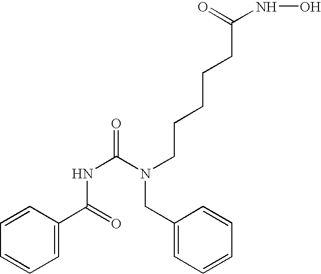 | 6-(3-Benzoyl-1-benzyl-ureido)-hexanoic acid hydroxyamide, |
| 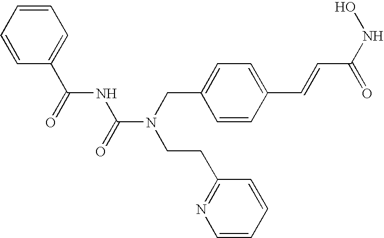 | 3-{4-[3-Benzoyl-1-(2-pyridin-2-yl-ethyl)-ureidomethyl]-pheny}-N-hydroxy-acrylamide, |
| 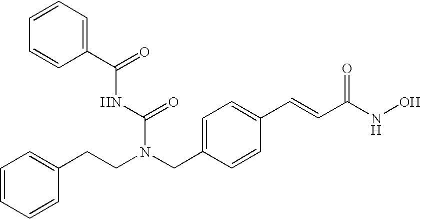 | 3-[4-(3-Benzoyl-1-phenethyl-ureidomethyl)-phenyl]-N-hydroxy-acrylamide, |
| 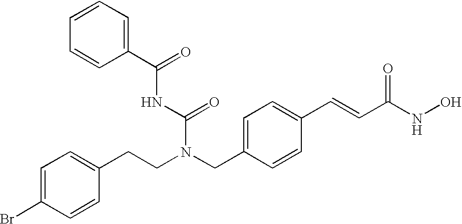 | 3-(4-{3-Benzoyl-1-[2-(4-bromo-phenyl)-ethyl]-ureidomethyl}-phenyl)-N-hydroxy-acrylamide, |
| 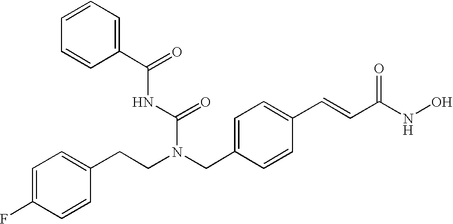 | 3-(4-{3-Benzoyl-1-[2-(4-fluoro-phenyl)-ethyl]-ureidomethyl}-phenyl)-N-hydroxy-acrylamide, |
| 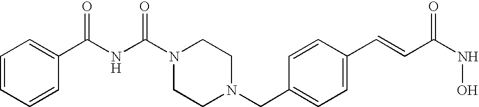 | N-{4-[4-(2-Hydroxycarbamoyl-vinyl)-benzyl]-piperazine-1-carbonyl}-benzamide, |
| 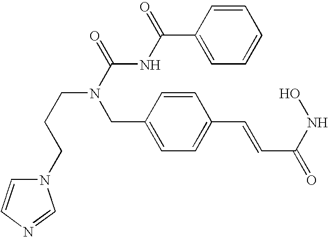 | 3-{4-[3-Benzoyl-1-(3-imidazol-1-yl-propyl)-ureidomethyl]-phenyl}-N-hydroxy-acrylamide, |
| 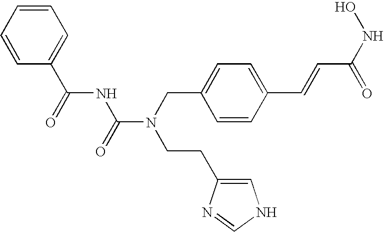 | 3-(4-{3-Benzoyl-1-[2-(1H-imidazol-4-yl)-ethyl]-ureidomethyl}-phenyl)-N-hydroxy-acrylamide, |
| 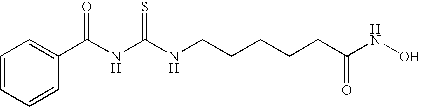 | 6-(3-Benzoyl-thioureido)-hexanoicacid hydroxyamide, |
| 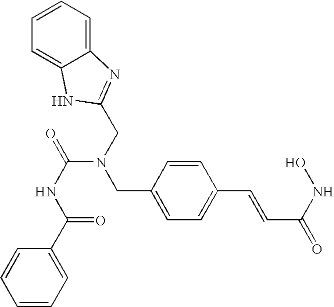 | 3-{4-[1-(1H-Benzoimidazol-2-ylmethyl)-3-benzoyl-ureidomethyl]-phenyl}-N-hydroxy-acrylamide, |
| 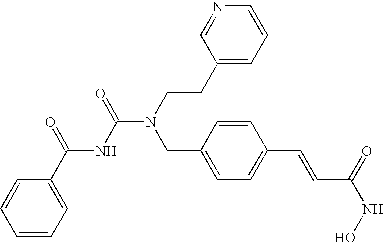 | 3-{4-[3-Benzoyl-1-(2-pyridin-3-yl-ethyl)-ureidomethyl]-phenyl}-N-hydroxy-acrylamide, |
| 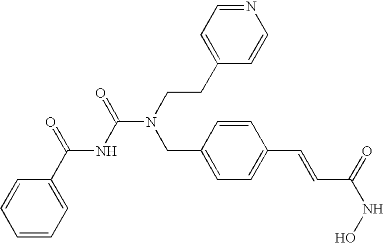 | 3-{4-[3-Benzoyl-1-(2-pyridin-4-yl-ethyl)-ureidomethyl]-phenyl}-N-hydroxy-acrylamide, |
| 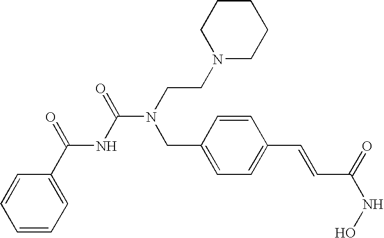 | 3-{4-[3-Benzoyl-1-(2-piperidin-1-yl-ethyl)-ureidomethyl]-phenyl}-N-hydroxy-acrylamide, |
| 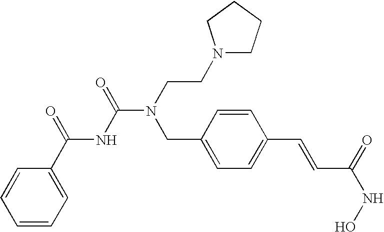 | 3-{4-[3-Benzoyl-1-(2-pyrrolidin-1-yl-ethyl)-ureidomethyl]-phenyl}-N-hydroxy-acrylamide |

or a pharmaceutically acceptable salt or prodrug thereof.

116. A compound according to claim 80 selected from the group consisting of

| | |
|---|---|
| 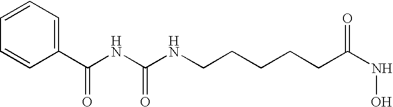 | 6-(3-Benzoyl-ureido)-hexanoic acidhydroxyamide, |
| 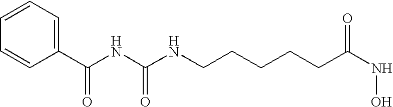 | 8-(3-Benzoyl-ureido)-octanoic acidhydroxyamide, |
| 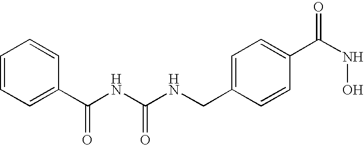 | 4-(3-Benzoyl-ureidomethyl)-N-hydroxy-benzamide, |
| 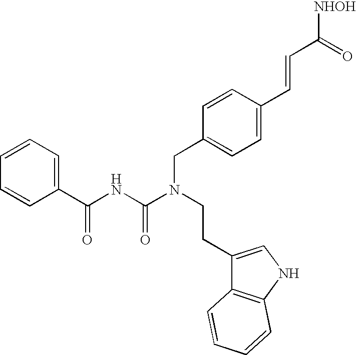 | 3-(4-{3-Benzoyl-1-[2-(1H-indol-3-yl)-ethyl]-ureidomethyl}-phenyl)-N-hydroxy-acrylamide, |
| 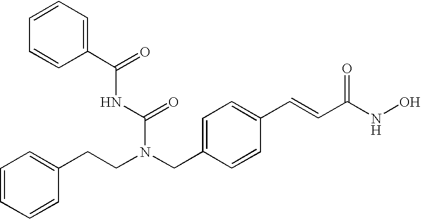 | 3-[4-(3-Benzoyl-1-phenethyl-ureidomethyl)-phenyl]-N-hydroxy-acrylamide, |
| 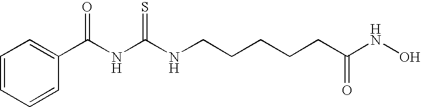 | 6-(3-Benzoyl-thioureido)-hexanoicacid hydroxyamide, |
| 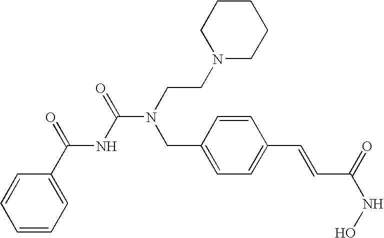 | 3-{4-[3-Benzoyl-1-(2-piperidin-1-yl-ethyl)-ureidomethyl]-phenyl}-N-hydroxy-acrylamide, |
| 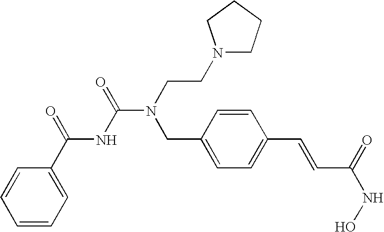 | 3-{4-[3-Benzoyl-1-(2-pyrrolidin-1-yl-ethyl)-ureidomethyl]-phenyl}-N-hydroxy-acrylamide, |

or a pharmaceutically acceptable salt or prodrug thereof.

117. A pharmaceutical composition including a compound according to claim 80 and a pharmaceutically acceptable diluent, excipient or carrier.

118. A method of treatment of a disorder caused by, associated with or accompanied by disruptions of cell proliferation and/or angiogenesis in a patient, the method including administration of a therapeutically effective amount of a compound according to claim 80 to the patient.

119. A method according to claim 118 wherein the disorder is a proliferative disorder.

120. A method according to claim 119 wherein the proliferative disorder is cancer.

121. A method according to claim 120 wherein the cancer is selected from breast cancer, lung cancer, ovarian cancer, prostate cancer, head and neck cancer, renal cancer, gastric cancer, colon cancer, pancreatic cancer and brain cancer.

122. A method of modifying deacetylase activity including contacting the deacetylase with a compound according to claim 80.

123. A method according to claim 122 wherein the deacetylase activity is histone deacetylase activity.

124. A method according to claim 123 wherein the deacetylase activity is class I histone deacetylase activity.

125. A method according to claim 123 wherein the histone deacetylase is HDAC1.

126. A method according to claim 123 wherein the histone deacetylase is HDAC8.

127. A method of treatment of a disorder that can be treated by the inhibition of deacetylase activity in a patient including administration of a therapeutically effective amount of a compound according to claim 80 to the patient.

128. A method according to claim 127 wherein the deacetylase activity is histone deacetylase activity.

129. A method of treatment of a disorder that is mediated by histone deacetylase activity in a patient including administration of a therapeutically effective amount of a compound according to claim 80 to the patient.

130. A method according to claim 127 wherein the disorder is selected from the group consisting of Proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, Intracerebral haemorrhage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Interstitial keratitis, Diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, Inflammatory bowel disease, Colitis Ulcerosa, Alcoholic hepatitis, Diabetes, Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, mania, depression and dementia; Cardiovascular Diseases including Heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.

131. A method for inhibiting cell proliferation including administration of an effective amount of a compound according to claim 80.

132. A method of treatment of a neurodegenerative disorder in a patient including administration of a therapeutically effective amount of a compound according to claim 80 to the patient.

133. A method according to claim 132 wherein the neurodegenerative disorder is Huntington's Disease.

134. A method of treatment of an inflammatory disease and/or immune system disorder in a patient including administration of a therapeutically effective amount of a compound according to claim 80 to the patient.

135. A method according to claim 134 wherein the inflammatory disease and/or immune system disorder is rheumatoid arthritis.

136. A method according to claim 134 wherein the inflammatory disease and/or immune system disorder is systemic lupus erythematosus.

137. A method of treatment of a proliferative disorder in patient including administration of a therapeutically effective amount of a compound according to claim 80 to the patient.

138. A method of treatment of cancer in patient including administration of a therapeutically effective amount of a compound according to claim 80 to the patient.

139. A method according to claim 138 wherein the cancer is a hematologic malignancy.

140. A method according to claim 139 wherein the hematologic malignancy is selected from the group consisting of B-cell lymphoma, T-cell lymphoma and leukemia.

141. A method according to claim 138 wherein the cancer is a solid tumor.

142. A method according to claim 141 wherein the solid tumor is selected from the group consisting of breast cancer, lung cancer, ovarian cancer, prostate cancer, head and neck cancer, renal cancer, gastric cancer, colon cancer, pancreatic cancer and brain cancer.

143. A method of induction of apoptosis of a cell including contacting the cell with an effective amount of a compound according to claim 80.

144. A method according to claim 124 wherein the histone deacetylase is HDAC1.

145. A method according to claim 124 wherein the histone deacetylase is HDAC8.

146. A method according to claim 128 wherein the disorder is selected from the group consisting of Proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, Intracerebral haemorrhage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Interstitial keratitis, Diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, Inflammatory bowel disease, Colitis Ulcerosa, Alcoholic hepatitis, Diabetes, Sjoegrens's syndrome, Multiple Sclerosis, Ankylosing spondylitis, Membranous glomerulopathy, Disco genic pain, Systemic Lupus Erythematosus; Disease involving angiogenesis including cancer, psoriasis, rheumatoid arthritis; Psychological disorders including bipolar disease, schizophrenia, mania, depression and dementia; Cardiovascular Diseases including Heart failure, restenosis and arteriosclerosis, Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.

147. A method according to claim 129 wherein the disorder is selected from the group consisting of Proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, Intracerebral hemorrhage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Interstitial keratitis, Diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, Inflammatory bowel disease, Colitis Ulcerosa, Alcoholic hepatitis, Diabetes, Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, mania, depression and dementia; Cardiovascular Diseases including Heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.
